(19) United States
(12) Patent Application Publication
(10) Pub. No.: US 2016/0068906 A1
(43) Pub. Date: Mar. 10, 2016

(54) METHOD OF SCREENING NEWBORNS FOR GENE VARIANTS

(71) Applicant: Baby Genes, Inc., Golden, CO (US)

(72) Inventors: Richard SJOGREN, Golden, CO (US); Jason W. MYERS, Golden, CO (US)

(21) Appl. No.: 14/480,499

(22) Filed: Sep. 8, 2014

(51) Int. Cl. C12Q 1/68

(52) U.S. Cl. CPC C12Q 1/6883; C12Q 2600/156

(57) ABSTRACT

Disclosed are methods, systems, and kits for screening a newborn infant for one or more gene variants comprising, obtaining a genomic DNA containing sample from the newborn infant; sequencing at least one target region of each of two or more genes selected from the group consisting of PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 in the genomic DNA; and screening for a gene variant from the sequenced target regions of each gene to identify gene variants present in the genomic DNA, wherein the sequencing does not include whole genome sequencing or whole exome sequencing.

### SUMMARY OF THE INVENTION

Disclosed herein, in certain embodiments, are methods for screening a newborn infant for one or more gene variants comprising, obtaining a genomic DNA containing sample from the newborn infant; sequencing at least one target region of each of two or more genes selected from the group consisting of PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 in the genomic DNA; and screening for a gene variant from the sequenced target regions of each gene to identify gene variants present in the genomic DNA, wherein the sequencing does not include whole genome sequencing or whole exome sequencing. In some embodiments, there are provided methods for screening a newborn infant for one or more gene variants comprising, obtaining a genomic DNA containing sample from the newborn infant; sequencing at least one target region of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 in the genomic DNA; and screening for a gene variant from the sequenced target regions of each gene to identify gene variants present in the genomic DNA, wherein the sequencing does not include whole genome sequencing or whole exome sequencing. In some embodiments, the one or more gene variants are associated with one or more diseases or disorders. In some embodiments, the infant is asymptomatic for a disease or disorder. In some embodiments, the method is completed in less than 96 hours. In some embodiments, the at least one target region of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 is sequenced. In some embodiments, the method further comprises sequencing at least one target region of one or more genes selected from the group consisting of MCCC1, MCCC2, HMGCL, HLCS, GCDH, SLC22A5, HADHB, ASS1, CBS, MTHFR, MTR, MTRR, MMADHC, PAH, FAH, DUOX2, PAX8, SLC5A5, TG, TPO, TSHB, TSHR, HBB, BTD, CFTR, GJB2, GJB3, GJB6, ADA, and IL2RG. In some embodiments, the method further comprises sequencing at least one target region of one or more genes selected from the group consisting of MLYCD, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, GALK1, GALC, GBA, NPC1, NPC2, GAA, GLA, IDUA, ABCD1, and NGLY1. In some embodiments, at least one target region of each of 3, 4, 5, 6, 7, 8, 9, 10, 11, 12, 13, 14, 15, 16, 17, 18, 19, 20 or more genes is sequenced. In some embodiments, the target region comprises all or a portion of an exon. In some embodiments, the target region comprises about 50 bases to about 1000 bases. In some embodiments, the target region comprises about 50, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500 or more bases. In some embodiments, two or more target regions for each gene are sequenced. In some embodiments, 2, 3, 4, 5, 6, 7, 8, 9, 10, 11, 12, 13, 14, 15, 16, 17, 18, 19, 20 or more target regions for each gene are sequenced. In some embodiments, the gene variants are selected from among gene variants listed in Table 5. In some embodiments, the target region that is selected comprises all or a portion of an exon encoding a portion of a gene selected from among the genes listed in Table 4. In some embodiments, the gene variants are selected from a group consisting of a splice site mutation, an in-frame mutation, a nonsense mutation, a mutation comprising an unknown nucleic acid base, and a frameshift mutation. In some embodiments, the gene variants are located in an exon, an intron, a splice site, a codon, a regulatory element, or a non-coding region. In some embodiments, the sample is a blood sample. In some embodiments, the blood sample is dried blood sample. In some embodiments, the sample is from a newborn infant between 0 and 72 hours after birth. In some embodiments, the sample is from a newborn infant less than 48 hours, less than 24 hours, less than 12, less than 6, less than 4, less than 2 hours, or less than 1 hour after birth. In some embodiments, the variant is identified less than 60 hours following collection of the sample. In some embodiments, the variant is identified less than 50 hours following collection of the sample. In some embodiments, the number of sequence reads per target region is 5, 6, 7, 8, 9, 10, 11, 12, 13, 14, 15, 16, 17, 18, 19, 20 or more. In some embodiments, the variant is identified using a computer software module. In some embodiments, the method further comprises repeating the method one or more times at predetermined intervals after birth of the newborn infant. In some embodiments, the method further comprises repeating the method at 4 days, 5 days, 6 days, 7 days, 8 days, 9 days, 10 days, 11 days, 12 days, 13 days, 14 days, 3 weeks, 4 weeks, or one month after birth of the newborn infant. In some embodiments, the method further comprises repeating the method prior to discharge of the newborn infant from a care facility after birth. In some embodiments, the newborn infant does not exhibit symptoms of a metabolic disease or condition. In some embodiments, the sample is from an infant receiving care in a newborn intensive care unit (NICU). In some embodiments, the method further comprises providing a report comprising a list of variants identified in the genomic DNA. In some embodiments, the report includes a list of diseases or disorders associated with each variant. In some embodiments, the method further comprises selecting the infant for diagnostic assay for the disease or disorder if a gene variant associated with the disease or disorder is identified. In some embodiments, the diagnostic assay comprises detecting a biomarker indicative of the disease or disorder associated with the gene variant identified. In some embodiments, the detecting is by mass spectrometry. In some embodiments, the detecting is with an antibody. In some embodiments, the disease or disorder is a metabolic disorder. In some embodiments, the metabolic disorder is an organic acid disorder. In some embodiments, the organic acid disorder is propionic acidemia (PROP), methylmalonic acidemia (MUT), isovaleric acidemia (IVA), 3-methylcrotonyl-CoA carboxylase deficiency (3-MCC), 3-hydroxy-3-methylglutaryl-CoA lyase deficiency (HMG), multiple carboxylase deficiency (MCD), beta-ketothiolase deficiency (βKT), or glutaric acidemia type I (GA1). In some embodiments, the metabolic disorder is a fatty acid oxidation disorder. In some embodiments, the fatty acid oxidation disorder is primary carnitine deficiency (CUD), medium-chain acyl-CoA dehydrogenase (MCAD) deficiency, very long-chain acyl-CoA dehydrogenase (VLCAD) deficiency, long chain 3-hydroxyacyl-CoA dehydrogenase (LCHAD) deficiency, or trifunctional protein deficiency (TFP). In some embodiments, the metabolic disorder is an amino acid disorder. In some embodiments, the amino acid disorder is argininosuccinic aciduria (ASA), citrullinemia (CIT) type I, maple syrup urine disease (MSUD), homocystinuria (HCY), phenylketonuria (PKU), or tyrosinemia (TYR I, II, III). In some embodiments, the disease or disorder is an endocrine disorder. In some embodiments, the endocrine disorder is congenital hypothyroidism (CH) or 21-hydroxylase deficiency (CAH). In some embodiments, the disease or disorder is a hemoglobin disorder. In some embodiments, the hemoglobin disorder is sickle cell disease, metheglobinemia beta-globin type, or beta thalassemia. In some embodiments, the beta thalassemia is thalassemia major or thalassemia intermedia. In some embodiments, the disease or disorder is biotinidase deficiency (BIOT), cystic fibrosis (CF), galactosemia type I, hearing loss, severe combined immunodeficiency (SCID), or X-linked severe combined immunodeficiency (SCID). In some embodiments, the hearing loss is nonsyndromic deafness, palmoplantar karatoderma, hystrix-like ichthyosis, Bart-Pumphrey syndrome, Vohwinkel syndrome, karatitis-ichthyosis-deafness (KID), erythrokeratodermia variabilis et progressive (EKVP), or Clouston syndrome. In some embodiments, the disease or disorder is malonyl-CoA decarboxylase deficiency (MAL), isobutyryl-CoA dehydrogenase (IBD) deficiency, 2-methylbutyryl-CoA dehydrogenase deficiency, 3-methylglutaconic aciduria (3MGA) type I, 3-methylglutaconic aciduria (3MGA) type V, 3-hydroxy-2-methylbutyryl-CoA dehydrogenase deficiency (2M3HBA), short-chain acyl-CoA dehydrogenase (SCAD) deficiency, 3-hydroxyacyl-CoA dehydrogenase deficiency (M/SCHAD), glutaric acidemia type II (GA2), glutaric acidemia type II (GA2), carnitine palmitoyltransferase I deficiency (CPT IA), carnitine palmitoyltransferase II deficiency (CPT II), carnitine-acylcarnitine translocase (CACT), arginase deficiency (ARG), citrullinemia type II (CIT II), hypermethioninemia (MET), disorders of biopterin regeneration, tyrosinemia (TYR I, II, III), alpha thalassemia (hemoglobin disorder-Var-Hb), galactosemia type II, or galactosemia type III. In some embodiments, the disease or disorder is X-linked adrenoleukodystrophy adrenomyeloneuropathy Addison disease (X-ALD), 2,4 dienoyl-CoA reductase deficiency, Pompe disease (GAA deficiency), Krabbe Disease, Gaucher disease (types I, II, & III), Fabry disease, mucopolysaccharidosis type I (MPS I), congenital disorder of deglycosylation type 1v, Niemann-Pick disease (type C1), or Niemann-Pick disease (type C2). In some embodiments, the disease or disorder is congenital adrenal hyperplasia (CAH), medium chain acyl-CoA dehydrogenase deficiency (MCAD), long chain 3-hydroxyacyl-CoA dehydrogenase deficiency (LCHAD), very long chain acyl-CoA dehydrogenase deficiency (VLCAD), beta-ketothiolase deficiency (BKD), isobutyryl CoA dehydrogenase deficiency (IBD), isovaleric acidemia (IVA), maple syrup urine disease (MSUD), methylmalonic acidemias (MMA/8 types), propionic acidemia (PROP), argininosuccinate lyase deficiency (ASA), or galactosemia. In some embodiments, there are provided methods of screening a newborn for one or more gene variants comprising, identifying a gene variant by sequencing at least one target region from each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8, in a genomic DNA containing sample from the newborn infant, wherein the sequencing does not include whole genome sequencing or whole exome sequencing.

In some embodiments there are provided methods of screening newborn infants for a newborn infant for one or more gene variants comprising (a) generating a genomic library pool from genomic DNA containing sample from a newborn infant; (b) performing a plurality of DNA sequencing reactions on the genomic library pool to determine the DNA sequence of at least one target region in each of two or more genes selected from the group consisting of PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8, wherein DNA containing the target regions is simultaneously sequenced to produce a plurality of sequencing reads for each target region, wherein the sequencing reactions do not comprise whole genome sequencing or whole exome sequencing; (c) identifying gene variants in the two or more genes by comparing the plurality of sequencing reads for each target region to a reference sequence; and (d) generating a report that all identified gene variants. In some embodiments, the generating a genomic library pool comprises amplifying the genomic DNA. In some embodiments, the generating a genomic library pool comprises: (a) fragmenting the isolated genomic DNA to produce fragmented genomic DNA; (b) ligating adaptors to the fragmented genomic DNA to produce adaptor-modified genomic DNA; and (c) amplifying the adaptor-modified genomic DNA. In some embodiments, the adaptor comprises a barcode. In some embodiments, the genomic DNA is amplified by polymerase chain reaction. In some embodiments, the adaptor-modified genomic DNA is amplified using oligonucleotide primers specific to the target region. In some embodiments, the oligonucleotide primers are labeled. In some embodiments, the one or more gene variants are associated with one or more diseases or disorders. In some embodiments, the infant is asymptomatic for a disease or disorder. In some embodiments, the method is completed in less than 96 hours. In some embodiments, the at least one target region of each of gene PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 is sequenced. In some embodiments, the method further comprises identifying a gene variant by sequencing at least one target region of one or more genes selected from the group consisting of MCCC1, MCCC2, HMGCL, HLCS, GCDH, SLC22A5, HADHB, ASS1, CBS, MTHFR, MTR, MTRR, MMADHC, PAH, FAH, DUOX2, PAX8, SLC5A5, TG, TPO, TSHB, TSHR, HBB, BTD, CFTR, GJB2, GJB3, GJB6, ADA, and IL2RG. In some embodiments, the method further comprises identifying a gene variant by sequencing at least one target region of one or more genes selected from the group consisting of MLYCD, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, GALK1, GALC, GBA, NPC1, NPC2, GAA, GLA, ID UA, ABCD1, and NGLY1. In some embodiments, at least one target region of each of 3, 4, 5, 6, 7, 8, 9, 10, 11, 12, 13, 14, 15, 16, 17, 18, 19, 20 or more genes is sequenced. In some embodiments, the target region comprises all or a portion of an exon. In some embodiments, the target region comprises about 50 bases to about 1000 bases. In some embodiments, the target region comprises about 50, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500 or more bases. In some embodiments, two or more target regions for each gene are sequenced. In some embodiments, 3, 4, 5, 6, 7, 8, 9, 10, 11, 12, 13, 14, 15, 16, 17, 18, 19, 20 or more target regions for each gene are sequenced. In some embodiments, the gene variants are selected from among gene variants listed in Table 5. In some embodiments, the target region that is selected comprises all or a portion of an exon encoding a portion of a gene selected from among the genes listed in Table 4. In some embodiments, the gene variants are selected from a group consisting of a splice site mutation, an in-frame mutation, a nonsense mutation, a mutation comprising an unknown nucleic acid base, and a frameshift mutation. In some embodiments, the gene variants are located in an exon, an intron, a splice site, a codon, a regulatory element, and a non-coding region. In some embodiments, the sample is a blood sample. In some embodiments, the blood sample is dried blood sample. In some embodiments, the sample is from a newborn infant between 0 and 72 hours after birth. In some embodiments, the sample is from a newborn infant less than 48 hours, less than 24 hours, less than 12, less than 6, less than 4, less than 2 hours, or less than 1 hour after birth. In some embodiments, the gene variant is identified less than 60 hours following collection of the sample. In some embodiments, the gene variant is identified less than 50 hours following collection of the sample. In some embodiments, the number of sequence reads per target region is 5, 6, 7, 8, 9, 10, 11, 12, 13, 14, 15, 16, 17, 18, 19, 20 or more. In some embodiments, the gene variant is identified using a computer software module. In some embodiments, the method further comprises repeating the method one or more times at predetermined intervals after birth of the newborn infant. In some embodiments, the method further comprises repeating the method at 4 days, 5 days, 6 days, 7 days, 8 days, 9 days, 10 days, 11 days, 12 days, 13 days, 14 days, 3 weeks, 4 weeks, or one month after birth of the newborn infant. In some embodiments, the method further comprises repeating the method prior to discharge of the newborn infant from a care facility after birth. In some embodiments, the newborn infant does not exhibit symptoms of a metabolic disease or condition. In some embodiments, the sample is from an infant receiving care in a newborn intensive care unit (NICU). In some embodiments, the method further comprises providing a report comprising a list of variants identified in the sample. In some embodiments, the report includes a list of diseases or disorders associated with each identified gene variant. In some embodiments, the method further comprises selecting the infant for diagnostic assay for a disease or disorder if a gene variant associated with the disorder is identified. In some embodiments, the diagnostic assay comprises detecting a biomarker indicative of the disease or disorder associated with the gene variant identified. In some embodiments, the detecting is by mass spectrometry. In some embodiments, the detecting is with an antibody. In some embodiments, the disease or disorder is a metabolic disorder. In some embodiments, the metabolic disorder is an organic acid disorder. In some embodiments, the organic acid disorder is propionic acidemia (PROP), methylmalonic acidemia (MUT), isovaleric acidemia (IVA), 3-methylcrotonyl-CoA carboxylase deficiency (3-MCC), 3-hydroxy-3-methylglutaryl-CoA lyase deficiency (HMG), multiple carboxylase deficiency (MCD), beta-ketothiolase deficiency (βKT), or glutaric acidemia type I (GA1). In some embodiments, the metabolic disorder is a fatty acid oxidation disorder. In some embodiments, the fatty acid oxidation disorder is primary carnitine deficiency (CUD), medium-chain acyl-CoA dehydrogenase (MCAD) deficiency, very long-chain acyl-CoA dehydrogenase (VLCAD) deficiency, long chain 3-hydroxyacyl-CoA dehydrogenase (LCHAD) deficiency, or trifunctional protein deficiency (TFP). In some embodiments, the metabolic disorder is an amino acid disorder. In some embodiments, the amino acid disorder is argininosuccinic aciduria (ASA), citrullinemia (CIT) type I, maple syrup urine disease (MSUD), homocystinuria (HCY), phenylketonuria (PKU), or tyrosinemia (TYR I, II, III). In some embodiments, the disease or disorder is an endocrine disorder. In some embodiments, the endocrine disorder is congenital hypothyroidism (CH) or 21-hydroxylase deficiency (CAH). In some embodiments, the disease or disorder is a hemoglobin disorder. In some embodiments, the hemoglobin disorder is sickle cell disease, metheglobinemia, beta-globin type, or beta thalassemia. In some embodiments, the Beta thalassemia is thalassemia major or thalassemia intermedia. In some embodiments, the disease or disorder is biotinidase deficiency (BIOT), cystic fibrosis (CF), galactosemia type I, hearing loss, severe combined immunodeficiency (SCID), or X-linked severe combined immunodeficiency (SCID). In some embodiments, the hearing loss is nonsyndromic deafness, palmoplantar karatoderma, hystrix-like ichthyosis, Bart-Pumphrey syndrome, Vohwinkel syndrome, karatitis-ichthyosis-deafness (KID), erythrokeratodermia variabilis et progressive (EKVP), or Clouston syndrome. In some embodiments, the disease or disorder is malonyl-CoA decarboxylase deficiency (MAL), isobutyryl-CoA dehydrogenase (IBD) deficiency, 2-methylbutyryl-CoA dehydrogenase deficiency, 3-methylglutaconic aciduria (3MGA) type I, 3-methylglutaconic aciduria (3MGA) type V, 3-hydroxy-2-methylbutyryl-CoA dehydrogenase deficiency (2M3HBA), short-chain acyl-CoA dehydrogenase (SCAD) deficiency, 3-hydroxyacyl-CoA dehydrogenase deficiency (M/SCHAD), glutaric acidemia type II (GA2), glutaric acidemia type II (GA2), carnitine palmitoyltransferase I deficiency (CPT IA), carnitine palmitoyltransferase II deficiency (CPT II), carnitine-acylcarnitine translocase (CACT), arginase deficiency (ARG), citrullinemia type II (CIT II), hypermethioninemia (MET), disorders of biopterin regeneration, tyrosinemia (TYR I, II, III), alpha thalassemia (Hemoglobin Disorder-Var-Hb), galactosemia type II, or galactosemia type III. In some embodiments, the disease or disorder is X-linked adrenoleukodystrophy, adrenomyeloneuropathy, Addison disease (X-ALD), 2,4 dienoyl-CoA reductase deficiency, Pompe disease (GAA deficiency), Krabbe Disease, Gaucher disease (types I, II, & III), Fabry disease, mucopolysaccharidosis type I (MPS I), congenital disorder of deglycosylation type 1v, Niemann-Pick disease (type C1), or Niemann-Pick disease (type C2). In some embodiments, the wherein the disease or disorder is congenital adrenal hyperplasia (CAH), medium chain acyl-COA dehydrogenase deficiency (MCAD), long chain 3 hydroxyacyl-COA dehydrogenase deficiency (LCHAD), very long chain acyl-COA dehydrogenase deficiency (VLCAD), beta-ketothiolase deficiency (BKD), isobutyryl COA dehydrogenase deficiency (IBD), isovaleric acidemia (IVA), maple syrup urine disease (MSUD), methylmalonic acidemias (MMA/8 types), propionic acidemia (PROP), argininosuccinate lyase deficiency (ASA), or galactosemia.

In some embodiments, there are provide computer-implemented systems including (a) a digital processing device comprising an operating system configured to perform executable instructions and a memory and (b) a computer program including instructions executable by the digital processing device to create an application comprising: (i) a software module configured to receive a plurality of sequence reads, the sequence reads obtained from sequencing target regions of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8; (ii) a software module configured to perform an alignment of the plurality of sequence reads to a reference sequence; (iii) a software module configured to identify gene variants and, optionally, characterize the gene variants as pathogenic or likely pathogenic or associated with a disease or disorder; and (iv) a software module configured to generate a report providing a list of the gene variants identified. In some embodiments, the system further comprises a sequence analyzer communicatively connected with the software module configured to receive a plurality of sequence reads, wherein the sequence analyzer is configured for sequencing a plurality of target regions to provide a plurality of sequence reads. In some embodiments, the system further comprises a database, in computer memory, of gene variants selected from the gene variants listed in Table 5.

In some embodiments, there are provided genetic screening platforms comprising: (a) a processor configured to provide an application comprising: (i) a software module configured to receive a plurality of sequence reads, the sequence reads obtained from sequencing target regions of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8; (b) a server processor configured to provide a server application comprising: (i) a database, in computer memory, of gene variants selected from the gene variants listed in Table 5; (ii) a software module configured to perform an alignment of the plurality of sequence reads to a reference sequence; (iii) a software module configured to identify gene variants and, optionally, characterize the gene variants as pathogenic or likely pathogenic or associated with a disease or disorder; and (iv) a software module configured to generate a report providing a list of the gene variants identified.

In some embodiments, there are provided non-transitory computer-readable storage media encoded with a computer program including instructions executable by a processor to create an application comprising: (a) a database, in computer memory, of gene variants selected from the gene variants listed in Table 5; (b) a software module configured to receive a plurality of sequence reads, the sequence reads obtained from sequencing target regions of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8; (c) a software module configured to perform an alignment of the plurality of sequence reads to a reference sequence; (d) a software module configured to identify gene variants and, optionally, characterize the gene variants as pathogenic or likely pathogenic or associated with a disease or disorder; and (e) a software module configured to generate a report providing a list of the gene variants identified.

In some embodiments, there are provided compositions comprising a collection of oligonucleotide primers for selective amplification of plurality of target regions of each of PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8.

In some embodiments, there are provided kits comprising a collection of oligonucleotide primers for sequencing of each of PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8. In some embodiments, the kit further comprises one or more reagents for performing a sequencing reaction.

### BRIEF DESCRIPTION OF THE DRAWINGS

FIG. 1 illustrates a process flow diagram of an exemplary method for targeted high-throughput screening of newborn samples.

### DETAILED DESCRIPTION

### Certain Terminology

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers change and particular information on the internet is removed, but equivalent information is known and is readily accessed, such as by searching the internet and/or appropriate databases. Reference thereto evidences the availability and public dissemination of such information. Generally, the procedures for antibody production and molecular biology methods are methods commonly used in the art. Such standard techniques are found, for example, in reference manual, such as, for example, Sambrook et al. (2000) and Ausubel et al. (1994).

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

As used herein, ranges and amounts are expressed as “about” a particular value or range. About also includes the exact amount. Hence “about 5 bases” means “about 5 bases” and also “5 bases.” In some embodiments, “about” includes an amount that would be expected to be within experimental error. In some embodiments, “about” means plus or minus 10% of the expressed value.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the terms “subject”, “individual” and “patient” are used interchangeably. None of the terms are to be interpreted as requiring the supervision of a medical professional (e.g., a doctor, nurse, physician's assistant, orderly, hospice worker).

The terms “target region” and “targeted region” are used interchangeably herein and refer to a region of a gene that contains one or more locations of relevant gene variants. In some embodiments, the target region is an exon of a gene associated with a disease or condition. In some embodiments, the target region is of a gene associated with a disease or disorder listed in Table 1. In some embodiments, the target region is of a gene listed in Table 2. In some embodiments, the target region is an exon listed in Table 4. In some embodiments, the target region contains one or more variant set forth in Table 5.

As used herein, a “reference genome” (also simply called “reference”) is any known sequence to which a sequence read is aligned and contains the wild type sequence. In some embodiments, the reference genome corresponds to all or only part of the genome. In some embodiments, the reference is a gene or a target region of a gene.

The terms “gene variant” and “genetic variant” are used interchangeably and refer to mutation in a gene sequence compared to a wild type sequence. In some embodiments the gene variant is associated with a disease or disorder. In some embodiments, a variant is a change of one base to one or more other bases, an insertion of one or more bases, or a deletion of one or more bases. In some embodiments, a variant occurs in one chromosome. In some embodiments, a variant occurs in both chromosomes.

The term “obtaining” as used herein with reference to a genomic DNA containing sample includes receiving the sample by a testing facility. In some embodiments, the sample is collected from a newborn infant by a third party health care practitioner using known techniques and is shipped to the testing facility.

### Overview

Provided herein are methods for the rapid screening of newborn infants for gene variants associated with inherited diseases and conditions using a high-throughput targeted genomics-based sequencing assay. In some embodiments, a report of the results of the screening is provided to the newborn's parents or caregiver within a few days of birth, allowing the parents to seek medical advice regarding diagnostic testing or medical intervention as quickly after birth as possible to avoid the development of potentially debilitating disease. In some embodiments, the methods provided identify pathogenic or likely pathogenic genetic variants in the genome of the infant within a few days of birth, e.g., 48-72 hours after birth.

In some embodiments, the newborn screening assay provides comprehensive coverage of genetic conditions recommended for screening of all newborn infants, regardless of whether the infant is exhibiting symptoms of a disease or disorder. In some embodiments, the newborns are asymptomatic and thus, the method provides a means of identifying those infants carrying potentially pathogenic gene variants for diagnostic testing or monitoring prior to the presentation of any symptoms. The assay is adaptable to the addition of new gene targets for screening. The methods provided herein do not require whole genomic or whole exome sequencing, and therefore provides a low-cost primary screening approach for all newborns of high accuracy and sensitivity. In some embodiments, the methods allow for stratification of at risk infants for diagnostic screening for a diseases or conditions, or further monitoring for development of clinical symptoms. In some embodiments, the methods provided herein are performed prior to or in conjunction with current newborn diagnostic screening methods. The present targeted genomic screening allows for a rapid analysis of sequencing results and return of the results to the patient or care provider. In some embodiments, the patient is selected for diagnostic testing based on the screening results. In some embodiments, the methods provided herein further comprise additional testing for diagnosis of a disease or condition.

Currently, primary screening for genetic conditions in newborn infants is accomplished using a metabolic test for each metabolic condition of interest that each measure the concentration of one or more analytes in blood samples obtained from infants shortly after birth. Because the newborn screening tests are prescribed as part of state-based public health programs, the number and type of genetic conditions varies by state and jurisdiction. In most instances, the test for each condition is a separate assay for a particular metabolite or enzymatic activity for each disease or condition. Thus, coverage of all recommended condition requires multiple assays, which increases the likelihood of testing errors and test sample contamination. Newborn screening of premature, low birth weight, or sick infants is complex and test parameters are not optimized for these patient groups. For example, newborn intensive care unit (NICU) infants are more likely to generate false positive or false negative results and repeated screening is often necessary to obtain acceptable results. In turn, because of inaccurate results, necessary or life-saving treatment may be delayed. Exemplary metabolic tests include tandem mass spectrometry (MS/MS), time resolved fluoro-immunoassay, isoelectric focusing (IEF), fluorometric assay, or real time polymerase chain reaction (rtPCR). Validity of each individual test is subject to confounding factors, such as when and how the sample was collected. Further, sample rejection rate is high due to potential analyte contamination or interference, e.g. due to uneven application or layering in the assay, contamination from other samples, alcohol, glove powder and other collection contamination sources (e.g., improper handling), improper drying of the sample, over-application of the sample, serum or tissue contamination, improper storage of the sample or aging of the sample. In addition, because of the variability in the emergence of phenotypic symptoms of the particular disease or condition, multiple samples often are needed for proper screening. For example, a primary test is performed on a sample collected 24-48 hours after birth and a follow-up test is perform on a sample collected about 10-14 days after birth. On average, results are acquired within 7-14 days of birth.

The current newborn screening approaches also preclude the screening for genetic conditions without a metabolic marker or analyte, or conditions with delayed onset phenotypes. In addition, adding conditions to a recommended testing panel is time consuming and expensive. For example, certain tests, such as the test for severe combined immunodeficiency (SCID), require a specialized assay with specific equipment. As a result, a major cost/benefit decision must be made when adding specialized assays, resulting in the lack of universal adoption. Using a single genomics-based test for primary screening avoids such costly decisions by providing a convenient way to add additional conditions by simple addition of target screening regions to the panel. Thus, in some embodiments, the present methods are tailored to the newborn screening requirements for each state or jurisdiction.

In some embodiments, the methods provided herein screen for gene variants associated with each disease in a panel of diseases as prescribed by a particular state or jurisdiction. In some embodiments, the methods provided herein screen for gene variants associated with each disease in a panel of diseases including congenital adrenal hyperplasia (CAH), medium chain acyl-CoA dehydrogenase deficiency (MCAD), long chain 3-hydroxyacyl-CoA dehydrogenase deficiency (LCHAD), very long chain acyl-CoA dehydrogenase deficiency (VLCAD), beta-ketohiolase deficiency (BKD), isobutyryl-CoA dehydrogenase (IBD) deficiency, isovaleric acidemia (IVA), maple syrup urine disease (MSUD), methylmalonic acidemias (MMA/8 types), propionic acidemia (PROP), argininosuccinate lyase deficiency (ASA), and galactosemia. In some embodiments, the disease or condition is one or more conditions listed in listed in Table 1. In some embodiments, the methods provided herein screen for gene variants associated with each core condition listed in Table 1. In some embodiments, the methods provided herein screen for gene variants associated with each core condition and each secondary condition listed in Table 1. In some embodiments, the methods provided herein screen for gene variants associated with each core condition, each secondary condition, and each added condition listed in Table 1. In some embodiments, the diseases or conditions included in the panel of diseases include the primary and secondary conditions recommended for screening by the American College of Genetics for the screening of all newborn infants. In some embodiments, the diseases or conditions panel includes diseases or conditions that are recommended, but not yet incorporated into state-based screening newborn screening programs. In some embodiments, the methods provided herein screen for gene variants associated with a disease or condition.

### Exemplary Methods

Provided herein are exemplary methods for screening genomic DNA containing samples from newborn infants for gene variants associated with or likely to be associated with a disease or condition. In some embodiments, the methods provided herein involve screening for the presence or absence of a gene variant that is pathogenic or likely pathogenic. The methods provided herein involve targeted sequencing of genomic DNA containing samples from newborns, and do not comprise whole genome or whole exome sequencing. In some embodiments, the methods provided herein involve screening for the presence or absence of a gene variant associated with a disease or condition. In some embodiments, the disease or condition is a disease or condition listed in Table 1. In some embodiments, the disease or condition is a metabolic disorder. In some embodiments, the disease or condition is an organic acid disorder, a fatty acid oxidation disorder, an amino acid disorder, an endocrine disorder, a hemoglobin disorder, or a combination of any of these disorders.

In some embodiments, the methods provided herein involve sequencing target regions of selected genes for the presence or absence of a gene variant that is pathogenic or likely pathogenic. In some embodiments, the methods provided herein involve sequencing target regions of selected genes for the presence or absence of a gene variant associated with a disease or disorder. In some embodiments, the methods provided herein involve sequencing target regions of selected genes for the presence or absence of a gene variant associated with a disease or disorder listed in Table 1. In some embodiments, a target region of a gene associated with a gene or disorder listed in Table 1 is screened. In some embodiments, a gene selected from among the genes listed in Table 2 is screened. In some embodiments, two or more genes selected from among the genes listed in Table 2 are screened. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more genes are screened.

In some embodiments, the method comprises sequencing all or a portion of a targeted region of a selected gene or panel of genes. In some embodiments, all or a portion of the targeted region of each selected gene is sequenced. In some embodiments, at least one targeted region of each selected gene is sequenced. In some embodiments, two or more targeted regions of each selected gene are sequenced. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more target regions of each selected gene is sequenced. In some embodiments, the targeted regions are located within an exon. In some embodiments, the exons are selected from exons listed in Table 4.

In some embodiments, the method comprises screening for one or more variants in a selected gene or panel of genes. In some embodiments, the method comprises screening for one or more variants in a selected gene or panel of genes selected from among the genes lists in Table 2.

In some embodiments, the steps of the method involve (a) generating a genomic library pool from a genomic DNA containing sample from a newborn infant; (b) performing a plurality of DNA sequencing reactions on the genomic library pool to determine the DNA sequence of at least one target region in each of two or more genes selected from the genes listed in Table 2, wherein the DNA encoding the target regions is simultaneously sequenced to produce a plurality of sequencing reads for each target region; (c) identifying gene variants in the two or more genes by comparing the plurality of sequencing reads for each target region to a reference sequence; and (d) generating a report that characterizes all or a subset of identified gene variants as pathogenic or likely pathogenic or associated with the disease or disorder. In some embodiments, the genomic DNA containing sample is an isolated genomic DNA isolated from a biological sample from the infant.

In some embodiments, a target region of a gene selected from a gene listed in Table 2 is sequenced. In some embodiments, a target region of 3, 4, 5, 6, 7, 8, 9, 10, 11, 12, 13, 14, 15, 16, 17, 18, 19, 20 or more genes listed in Table 2 is selected. In some embodiments, a target region of a gene selected from PCCA, PCCB, MUT, MMAA, MMAB, MMADHC, MCEE, IVD, MCCC1, MCCC2, HMGCL, HLCS, ACAT1, GCDH, SLC22A5, ACADM, ACADVL, HADHA, HADHB, ASL, ASS1, BCKDHA, BCKDHB, DBT, DLD, CBS, MTHFR, MTR, MTRR, MMADHC, PAH, FAH, DUOX2, PAX8, SLC5A5, TG, TPO, TSHB, TSHR, CYP21A2, HBB, BTD, CFTR, GALT, GJB2, GJB3, GJB6, ADA, IL2RG, MLYCD, ACAD8, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, GALK1, GALC, GBA, NPC1, NPC2, GAA, GLA, ID UA, ABCD1, and NGLY1 is sequenced. In some embodiments, a target region of 3, 4, 5, 6, 7, 8, 9, 10, 11, 12, 13, 14, 15, 16, 17, 18, 19, 20 or more genes selected from PCCA, PCCB, MUT, MMAA, MMAB, MMADHC, MCEE, IVD, MCCC1, MCCC2, HMGCL, HLCS, ACAT1, GCDH, SLC22A5, ACADM, ACADVL, HADHA, HADHB, ASL, ASS1, BCKDHA, BCKDHB, DBT, DLD, CBS, MTHFR, MTR, MTRR, MMADHC, PAH, FAH, DUOX2, PAX8, SLC5A5, TG, TPO, TSHB, TSHR, CYP21A2, HBB, BTD, CFTR, GALT, GJB2, GJB3, GJB6, ADA, IL2RG, MLYCD, ACAD8, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, GALK1, GALC, GBA, NPC1, NPC2, GAA, GLA, IDUA, ABCD1, and NGLY1 is sequenced.

In some embodiments, a target region of a gene selected from PCCA, PCCB, MUT, MMAA, MMAB, MMADHC, MCEE, IVD, MCCC1, MCCC2, HMGCL, HLCS, ACAT1, GCDH, SLC22A5, ACADM, ACADVL, HADHA, HADHB, ASL, ASS1, BCKDHA, BCKDHB, DBT, DLD, CBS, MTHFR, MTR, MTRR, MMADHC, PAH, FAH, DUOX2, PAX8, SLC5A5, TG, TPO, TSHB, TSHR, CYP21A2, HBB, BTD, CFTR, GALT, GJB2, GJB3, GJB6, ADA, and IL2RG is sequenced. In some embodiments, a target region of 3, 4, 5, 6, 7, 8, 9, 10, 11, 12, 13, 14, 15, 16, 17, 18, 19, 20 or more genes selected from PCCA, PCCB, MUT, MMAA, MMAB, MMADHC, MCEE, IVD, MCCC1, MCCC2, HMGCL, HLCS, ACAT1, GCDH, SLC22A5, ACADM, ACADVL, HADHA, HADHB, ASL, ASS1, BCKDHA, BCKDHB, DBT, DLD, CBS, MTHFR, MTR, MTRR, MMADHC, PAH, FAH, DUOX2, PAX8, SLC5A5, TG, TPO, TSHB, TSHR, CYP21A2, HBB, BTD, CFTR, GALT, GJB2, GJB3, GJB6, ADA, and IL2RG is sequenced.

In some embodiments, a target region of a gene one or more additional genes is sequenced. In some embodiments, the one or more additional genes is selected from among MLYCD, ACAD8, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, and GALK1. In some embodiments, the one or more additional genes is selected from among GALC, GBA, NPC1, NPC2, GAA, GLA, IDUA, ABCD1, and NGLY1.

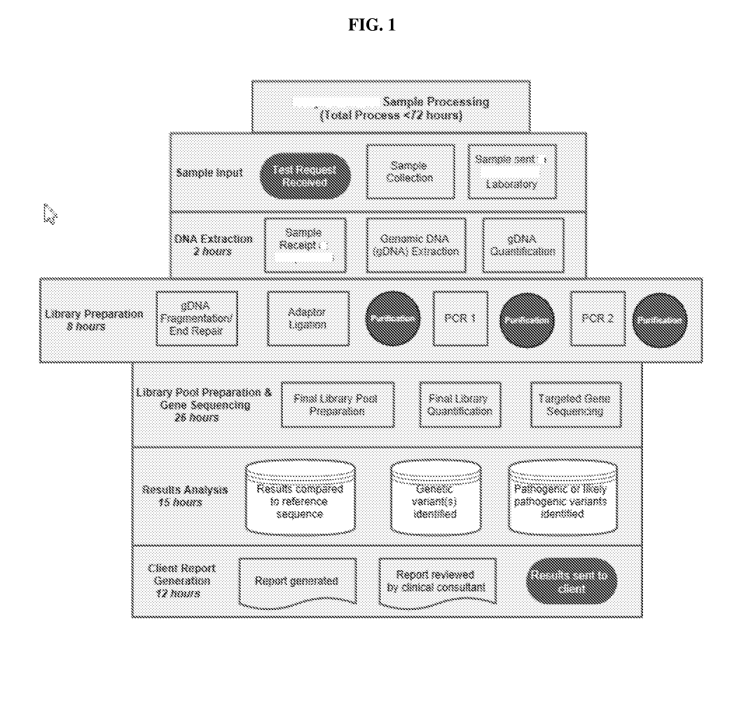

An exemplary illustration of the steps of the method is provided in FIG. 1. In some embodiments, all or some of the steps illustrated are performed. In some embodiments, the step performed include all or some of the following 1) sample input (e.g. receiving and preparation of the biological sample from a newborn infant), 2) genomic DNA extraction, 3) genomic library preparation, 4) genomic library pool preparation and gene sequencing, 5) results analysis, and 6) client report generation. In some embodiments, selected steps (e.g., results analysis, including sequence alignment, identification of variants, classification of variants) are performed with a computer system. In some embodiments, the results of method are used by a doctor in determining a diagnosis of the newborn infant.

In some embodiments, the biological sample includes genomic DNA of a newborn infant. In some embodiments, the genomic DNA is in the form of genomic segments of chromosomes. In some embodiments, genomic DNA is in the form of intact chromosomes. In some embodiments, the biological sample contains cells from the newborn infant. In some embodiments, the biological sample is a fluid or a tissue sample. Biological samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular embodiments, the sample is from a fluid or tissue that is part of, or associated with, the lymphatic system or circulatory system. In some embodiments, the sample is a blood sample that is a venous, arterial, peripheral, tissue, cord blood sample.

In some embodiments, the samples are obtained from the subject by any suitable means of obtaining the sample using well-known and routine clinical methods. Procedures for obtaining fluid samples from a subject are well-known. For example, procedures for drawing and processing whole blood and lymph are well-known and are employed to obtain a sample for use in the methods provided. In some embodiments, the sample is a blood sample obtained from the heel puncture of the newborn infant.

In some embodiments, for collection of a blood sample, the blood is dried on an absorbable medium such as a filter paper.

In some embodiments, for collection of a blood sample, an anti-coagulation agent (e.g. EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

Methods for the isolation of nucleic acids from cells contained in tissue or fluid samples are well-known in the art. In particular embodiments, the genomic DNA is isolated from cells contained in a blood sample collected from the newborn infant. In some embodiments, the genomic DNA extracted from the sample is quantified following extraction.

In some embodiments, the genomic DNA of the sample is fragmented, e.g., by sonication or other suitable methods to obtain smaller genomic segments. In some embodiments, genomic segments of about 200 to about 1000 bases long are generated. In some embodiments, genomic segments of less than about 200 bases long are generated. In some embodiments, genomic segments of greater than 1000 bases long are generated. In some embodiments, the ends of the fragmented genomic DNA are then prepared for adaptor ligation. Methods for end repair of fragments genomic DNA are well-known in the art. In some embodiments, the ends of the fragmented genomic DNA are blunted to prepare for adaptor ligation.

In some embodiments, the genomic segments are tagged with a barcode or multiplex identifier (MID). In some embodiments, a sequence of 10 bases are added (e.g., using a ligase) to the end of a genomic segment. In some embodiments a sequence of 10 bases is added using the primers provided in an ILLUMINA Index 2 Barcode Adaptor reaction packet. In this manner, segments from various samples are sequenced in parallel during a same sequencing run using the ID to multiplex. In some embodiments, the ID is read as part of a sequence read, and reads with the same ID are attributed to a same sample and analyzed as a group.

In some embodiments, the percentage of genomic segments representing a selected target region in the genomic sample is increased. In some embodiments, the percentage of genomic segments representing two or more selected target regions in the genomic sample is increased. In some embodiments, the percentage is increased by amplifying and/or enriching the sample for DNA from one or more targeted regions of the genome. In some embodiments, the resulting amplified sample is referred to as a target-increased sample. In some embodiments, a target region is selected from a gene listed in Table 2. In some embodiments, two or more target regions are selected from a gene listed in Table 4. In some embodiments, the target region is about a few hundred bases, e.g., 150-250 bases, 150-400 bases, or 200-600 bases.

In some embodiments, the addition of a sample-specific ID occurs at different steps of the method. For example, in some embodiments, the ID is added after the amplification/enrichment step and then the samples are mixed together. In this way, the different samples are amplified or enriched for different target regions. In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more target regions are used.

In some embodiments, forward and reverse primers are used to amplify a target region. In some embodiments, the forward and reverse primers are selected from various lengths, e.g., about 15-30 bases long. In some embodiments, the set of forward and reverse primers only amplify one part of the genome. Methods are available in the art for determining optimal primer length and specificity for amplification of genomic DNA segments.

In some embodiments, probes are used to capture genomic segments that correspond to the target region (i.e., enrichment of the target regions). In some embodiments, probes that are designed to hybridize to the target region are placed on a surface. Then, the genomic segments are placed over the surface and the segments of the target region are preferentially be hybridized. For example, a microarray with the probes are constructed, and the segments washed over the microarray. In some embodiments, the probes are about 25-75 bases long for a target region of about 200-550 bases long. As the probe can capture either end of a genomic segment, the segments, in some embodiments, for example, span a region of 200 bases to about 550 bases for genomic segments of up to 250 bases. Methods are available in the art for determining optimal length and specificity of nucleic acid probes for enrichment of genomic DNA segments

In another embodiment, both amplification and enrichment are performed. In some embodiments, the prepared amplified and/or enriched genomic DNA containing various targeted regions is referred to as the genomic library pool.

In some embodiments, the prepared genomic library pool is then sequenced. Sequence reads are determined from amplified and/or enriched genomic segments in the sample. In some embodiments, in the sequencing process, the clones of a same segment created in an amplification process are sequenced separately and optionally, counted later. In some embodiments, about 3,000 reads per sample are obtained. The number of reads depends on a number of factors, such as the size of the sample, amplification of the target region, and the bandwidth of the sequencing process (i.e., how much sequencing the apparatus is set for, e.g., how many beads are used). In some embodiments, not all of the segments in a sample are sequenced. In some embodiments, the sequence reads are about 150-250 bases long. One skilled in the art will appreciate the varied techniques available for performing the sequencing. In some embodiments, the sequencing is performed using an ILLUMINA MISEQ genome sequencer.

The sequencing process is performed by various techniques in various embodiments. In some embodiments, the fragments are amplified during the sequencing process. Where amplification was used to create a target region-increased sample, this amplification would be a second amplification step. The second amplification provides a stronger signal (e.g., a fluorescent signal corresponding to a particular base: A, C, G, or T) than if the second amplification was not performed and, the different amplicons do not result in separate sequence reads.

In some embodiments, the amplified genomic fragments (e.g., where amplification occurred in a solution) are each be attached to a bead. In some embodiments, the attached fragment is then amplified on the bead, and one sequence read is obtained from each bead. In some embodiments, for those that use a surface, a fragment is attached to a surface and then amplified to create a single cluster on the surface. In some embodiments, a single sequence read is obtained for each cluster. In some embodiments, a sequence read is for an entire length of a genomic segment, part of one end, or part of both ends.

In some embodiments, a sequence read includes the bases correspond to the actual segment and optionally the bases corresponding to a sample-specific ID and/or unique sequence tags (e.g., 25 bases long) that were used as part of the sequencing. In some embodiments, the unique sequence tags include part of an adapter that is ligated to the end of a fragment for receiving a universal primer, and part of the adapter is read during the sequencing.

In some embodiments, a plurality of sequence reads are aligned to a target region of a reference genome. By aligning, the process compares the sequence reads to the target region to determine the number of variations between the sequence read and the target region. A perfect match would show no variations. In some embodiments, a portion or all of the sequence reads obtained are used in the alignment process. For example, if the length of a read is too short or too long, then it is removed before alignment.

In some embodiments, the alignment is made so as to minimize the number of variations between the sequence read and the target region. In some embodiments, the sequence read is smaller than the target region or larger. In some embodiments, where the sequence read is larger, the number of variations is counted only in the target region.

In some embodiments, the reads are aligned to a target region only, thereby saving computational effort. As the alignment is specific to only the one or more target region(s), the alignment is performed in a short amount of time as the entire genome does not have to be searched. Also, as the percentage of segments corresponding to a target region is increased, a substantial number of the reads should match favorably to the target region (e.g., relatively few variations).

In some embodiments, where multiple target regions are used, a sequence read is compared to each target region, and the target region that provides the best alignment is identified. In some embodiments, the different target regions are different genes or different exons with a gene. Thus, in some embodiments, the exon with the best alignment is identified.

In some embodiments, where a barcode or ID is used, it is removed before aligning. In some embodiments, where a barcode or ID is used, it is not removed before aligning. In some embodiments, the barcode or ID is used to organize all of the reads for a particular sample into one group. In this manner, mutations from other samples do not impact the analysis of the present sample. This grouping is referred to as demultiplexing. In some embodiments, each sample is aligned to a different reference genome or different part of the reference genome. As different samples may have different target regions, the ID is used to determine which target region(s) of a reference genome should be compared for the alignment.

In some embodiments, sequence reads that differ from a target region by more than a first threshold number of variations are discarded from analysis for the target region. In some embodiments, where the number of variations is more than the threshold, it is an indication that the genomic segment corresponding to the sequence read did not come from the target region, given that the read was so different. In some embodiments, an allowance is made for some variations, so that a later analysis is used to identify mutations, which otherwise would be missed.

Example values for the threshold are 5-10 bases. In some embodiments, the threshold is dependent on the size of the target region. For example, in some embodiments, where the target region is about 200 bases, then the number of variations is capped at about 20 bases, or about 10%. If the target region was 150 bases, then the threshold could be 15 bases.

In some embodiments, for each target region, the reads that have less than or equal to the threshold are identified, e.g., as a group. In some embodiments, this group of reads is then analyzed further in relation to the target region. In some embodiments, where a read satisfies the threshold for more than one target region, it is then added to both groups. Such a read is tracked such that it is not ultimately counted as a mutation for more than one target region.

In some embodiments, accuracy is evaluated in multiple stages, through the library preparation and sequencing of gDNA of well-established samples (Coriell Institute NA12878) with known genomic variants. The ARCHER pipeline (using publicly available tools and algorithms, e.g., BWA, samtools, and freebayes) is used to identify or “call” all genomic variants in comparison to the human reference genome. All detected variants for each sample are compared to the known variants to determine if the sequenced variant(s) agree with the reported variant(s). These known mutations are available for data analysis on the GeT-RM database. The accuracy data is then used to evaluate the sensitivity. The sensitivity data, of the analyzed sample set, is accepted only if at least 85% sensitivity is achieved with 95% confidence interval. Variants are called for replicates of samples, run either on the same sequence runs or multiple sequence runs, and all detected variants for each replicate sample are compared to determine the repeatability and precision of variant calling. The variant calls are accepted if they agree 90% between replicate samples. These verification samples are run on a regular basis to ensure that the system is performing at an acceptable sensitivity/specificity level. Client samples are run similar to the verification samples in that gDNA extraction, library preparation and sequencing are all the same. The resultant raw sequence reads are aligned to the human genome (GCRh37) using Burrows-Wheeler Aligner (bwa) and variants are called using samtools and freebayes. Variants are then filtered for quality and binned to target regions.

In some embodiments, once the variants have been identified within a particular target region, the variants are further characterized as pathogenic or likely pathogenic based on factors, including, but not limited to, current knowledge of the particular gene function or association with a particular disease or condition, nature of the mutation, or a combination thereof.

In some embodiments, the identified variant or variants are compared to known databases of variants. In some embodiments, the variants are for the same target region. In some embodiments, the variants occur for a certain population or subpopulation of people, which is different than the reference genome used.

In some embodiments, the sequence reads from the target region are used to identify mutations in the target region. In some embodiments, the frequency of each variation is determined. For example, for a particular position in a target region, the number of times a G nucleotide variation appears instead of a normal or wild type A nucleotide is counted. A percentage of times the G mutation is seen is determined from the total reads that aligned to that position. In one embodiment, the percentage for a particular variation is required to be greater than a threshold (abundance filter) to be considered an actual mutation. In some embodiments, variations that occur together are identified. In some embodiments, variations that occur together are categorized as part of a same mutation.

In some embodiments, a report is generated which summarizes the identified variants. In some embodiments, the report lists the genes in which each variant was found. In some embodiments, the report lists the genomic location (e.g., chromosome number and numerical location) in which each variant was found. In some embodiments, the report lists the type variant (e.g., single nucleotide change or deletion). In some embodiments, the report lists the identity of the variant (e.g., an A to G mutation). In some embodiments, the report provides information on which variants are pathogenic or likely to be pathogenic. In some embodiments, the report provides information on which variants are associated with a disease of condition. In some embodiments, the disease or condition is a disease or condition listed in Table 1. In some embodiments, the report provides information on the disease or condition, including, but not limited to symptoms, pathology, diagnostic testing, and treatment. In some embodiments, the report provides recommendations for diagnostic genetic testing or diagnostic metabolic testing.

In some embodiments, the time from receipt of the newborn sample to the generation of a report is less than 96 hours. In some embodiments, the time from receipt of the sample to the generation of a report is less than 72 hours. In some embodiments, the time from receipt of the sample to the generation of a report is less than 48 hours.

### Exemplary Computer Systems and Software Modules

In some embodiments, there are provide computer-implemented systems including (a) a digital processing device comprising an operating system configured to perform executable instructions and a memory and (b) a computer program including instructions executable by the digital processing device to create an application comprising: (i) a software module configured to receive a plurality of sequence reads, the sequence reads obtained from sequencing target regions of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8; (ii) a software module configured to perform an alignment of the plurality of sequence reads to a reference sequence; (iii) a software module configured to identify gene variants and, optionally, characterize the gene variants as pathogenic or likely pathogenic or associated with a disease or disorder; and (iv) a software module configured to generate a report providing a list of the gene variants identified. In some embodiments, the system further comprises a sequence analyzer communicatively connected with the software module configured to receive a plurality of sequence reads, wherein the sequence analyzer is configured for sequencing a plurality of target regions to provide a plurality of sequence reads. In some embodiments, the system further comprises a database, in computer memory, of gene variants selected from the gene variants listed in Table 5.

### Digital Processing Device

In some embodiments, the computer-implemented systems described herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® P53®, Sony® P54®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Computer Program

In some embodiments, the computer-implemented systems disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the computer-implemented systems disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the computer-implemented systems disclosed herein include one or more databases, or use of the same. In view of the disclosure provided herein, those of skill in the art will recognize that many databases are suitable for storage and retrieval of gene variant information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices. In some embodiments, the database includes gene variants of target regions of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 that are associated with a disease or disorder. In some embodiments, the database includes target regions of all or a subset of genes from the genes provided in Table 2. In some embodiments, the database includes the gene variants listed in Table 5.

In some embodiments, there are provided genetic screening platforms comprising: (a) a processor configured to provide an application comprising: (i) a software module configured to receive a plurality of sequence reads, the sequence reads obtained from sequencing target regions of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8; (b) a server processor configured to provide a server application comprising: (i) a database, in computer memory, of gene variants selected from the gene variants listed in Table 5; (ii) a software module configured to perform an alignment of the plurality of sequence reads to a reference sequence; (iii) a software module configured to identify gene variants and, optionally, characterize the gene variants as pathogenic or likely pathogenic or associated with a disease or disorder; and (iv) a software module configured to generate a report providing a list of the gene variants identified.

In some embodiments, there are provided non-transitory computer-readable storage media encoded with a computer program including instructions executable by a processor to create an application comprising: (a) a database, in computer memory, of gene variants selected from the gene variants listed in Table 5; (b) a software module configured to receive a plurality of sequence reads, the sequence reads obtained from sequencing target regions of each of genes PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8; (c) a software module configured to perform an alignment of the plurality of sequence reads to a reference sequence; (d) a software module configured to identify gene variants and, optionally, characterize the gene variants as pathogenic or likely pathogenic or associated with a disease or disorder; and (e) a software module configured to generate a report providing a list of the gene variants identified.

### Exemplary Diseases and Conditions for Monitoring

Organic Acid Disorders

Organic acid disorders result from enzyme deficiencies involved in the catabolism any of a number of organic compounds and metabolites. Organic acid disorders are those conditions that lead to an abnormal buildup of particular acids known as organic acids. Abnormal levels of organic acids in the blood (organic acidemia), urine (organic aciduria), and tissues can be toxic and can cause serious health problems. Present screening tests for organic acid disorders are MS/MS detection of acylcarnitines. Currently a diagnosis is confirmed with quantitative acylcarnitines, organic acids, enzyme assay and/or mutation analysis.

In some embodiments, the organic acid disorder is propionic acidemia (PROP), methylalonic acidemia (MUT), isovaleric acidemia (IVA), 3-methylcrotonyl-CoA carboxylase deficiency (3-MCC), 3-hydroxy-3-methylglutaryl-CoA lyase deficiency (HMG), multiple carboxylase deficiency (MCD), beta-ketothiolase deficiency (BKT), or glutaric acidemia type I (GA1).

In some embodiments, the organic acid disorder is malonyl-CoA decarboxylase deficiency (MAL), isobutyryl-CoA dehydrogenase (IBD) deficiency, 2-methylbutyryl-CoA dehydrogenase deficiency, 3-methylglutaconic aciduria (3MGA) type I, 3-methylglutaconic aciduria (3MGA) type V, 3-hydroxy-2-methylbutyryl-CoA dehydrogenase deficiency (2M3HBA).

In some embodiments, a genomic sample from a newborn is screened according to the methods provided herein for a gene variant associated with an organic acid disorder. In some embodiments, a genomic sample from a newborn is screened according to the methods provided herein for a gene variant in at least one target region of at least one gene selected from the group consisting of PCCA, PCCB, MUT, MMAA, MMAB, MMADHC, MCEE, IVD, MCCC1, MCCC2, HMGCL, HLCS, ACAT1, and GCDH. In some embodiments, a genomic sample from a newborn is screened according to the methods provided herein for a gene variant in at least one target region of at least one gene selected from the group consisting of MLYCD, ACAD8, ACADSB, AUH, DNAJC19, OPA3, TAZ, HSD17B1OH. In some embodiments, the methods provided further include a diagnostic test for an organic acid disorder. In some embodiments, the methods further include a diagnostic test for an organic acid disorder if a gene variant is detected in at least one target region of at least one gene selected from the group consisting of PCCA, PCCB, MUT, MMAA, MMAB, MMADHC, MCEE, IVD, MCCC1, MCCC2, HMGCL, HLCS, ACAT1, and GCDH. In some embodiments, the methods further include a diagnostic test for an organic acid disorder if a gene variant is detected in at least one target region of at least one gene selected from the group consisting of MLYCD, ACAD8, ACADSB, AUH, DNAJC19, OPA3, TAZ, HSD17B1OH. In some embodiments, the diagnostic test includes an acylcarnitine profile analysis of a biological sample from the newborn.

Beta-Ketothiolase Deficiency (BKD)

Beta-ketothiolase deficiency is an inherited disorder in which the body cannot effectively process a protein building block (amino acid) called isoleucine. Signs and symptoms typically appear between the ages of six to 24 months. Episodes called ketoacidotic attacks may occur causing symptoms such as vomiting, dehydration, difficulty breathing, extreme lethargy, and occasionally seizures. Infections, fasting, or increased intake of protein rich foods frequently triggers these ketoacidotic attacks. Attacks can also lead to coma. Present screening tests include assays for elevated levels of tiglylcarnitine (C5:1) and 3-hydroxyisovalerylcarnitine (C5OH).

In some embodiments, a genomic sample from a newborn is screened according to the methods provided herein for at least one gene variant in at least one target region of the acetyl-CoA acetyltransferase 1 (ACAT1) gene at chromosome 11q22.3. In some embodiments, the methods provided further include a diagnostic test for BKD. In some embodiments, the methods further include a diagnostic test for BKD if at least one gene variant in at least one target region of ACAT1 is detected. In some embodiments, the diagnostic test includes an acylcarnitine profile analysis of a biological sample from the newborn. In some embodiments, the diagnostic test includes analysis of tiglylcarnitine (C5:1) and/or 3-hydroxyisovalerylcarnitine (C5OH) levels in a biological sample from the newborn.

Isobutyryl CoA Dehydrogenase Deficiency (IBD)

Isobutyryl-CoA dehydrogenase (IBD) deficiency is a condition that disrupts the breakdown of certain proteins. In particular, patients with IBD deficiency have inadequate levels of an enzyme that helps break down the amino acid valine. Most effected individuals do not experience symptoms. A few children with IBD deficiency have developed features such as a weakened and enlarged heart, weak muscle tone, developmental delay, and anemia. IBD is currently detected by measuring elevated levels of isovalerylcarnitine (C5).

In some embodiments, a genomic sample from a newborn is screened according to the methods provided herein for at least one gene variant in at least one target region of the acyl-CoA dehydrogenase family, member 8 (ACAD8) gene at chromosome 11q25. In some embodiments, the methods provided further include a diagnostic test for IBD. In some embodiments, the methods further include a diagnostic test for IBD if at least one gene variant in at least one target region of ACAD8 is detected. In some embodiments, the diagnostic test includes an acylcarnitine profile analysis of a biological sample from the newborn. In some embodiments, the diagnostic test includes analysis of isovalerylcarnitine (C5) levels in a biological sample from the newborn.

Isovaleric Acidemia (IVA)

Isovaleric acidemia is a rare disorder in which the body is unable to process certain proteins properly. Patients with isovaleric acidemia have inadequate levels of an enzyme that helps break down the amino acid called leucine. Cases vary from mild to life threatening and in severe cases the features of the disorder become apparent within days after birth. Symptoms include poor feeding, vomiting, seizures, lethargy, coma and possibly death. An odor of sweaty feet is present with acute illness. IVA is currently detected by measuring elevated levels of C4.

In some embodiments, a genomic sample from a newborn is screened according to the methods provided herein for at least one gene variant in at least one target region of the isovaleryl-CoA dehydrogenase (IVD) gene at chromosome 15q14-q15. In some embodiments, the methods provided further include a diagnostic test for IVA. In some embodiments, the methods further include a diagnostic test for IVA if at least one gene variant in at least one target region of IVD is detected. In some embodiments, the diagnostic test includes an acylcarnitine profile analysis of a biological sample from the newborn. In some embodiments, the diagnostic test includes analysis of isobutyryl (C4) levels in a biological sample from the newborn.

Fatty Acid Disorders

Fatty acid disorders are those disorders in which an enzyme deficiency prevents the body from converting certain fats to energy. Mitochondrial beta-oxidation of fatty acids is important in the body's ability to produce energy during fasting. In infants, a “fasting” state can be produced in as little as four hours. Fatty acids must be transported into the cytoplasm and then into the mitochondria for oxidation; carnitine is required for these transport steps. Once in the mitochondria, fatty acid chains 4-18 carbons in length must be oxidized, two carbons at a time, each reaction using a chain-specific enzyme, before ketogenesis can occur. There are over 20 individual steps in beta oxidation some with multiple enzyme complexes. An enzyme block or deficiency anywhere in this process or a carnitine deficiency results in hypoketotic hypoglycemia and tissue damage related to the toxic accumulation of unoxidized fatty acids. At least 16 separate enzyme disorders have been identified within this oxidation process, which are currently identified by measuring the accumulation of various acylcarnitines.

In some embodiments, the fatty acid disorder is primary carnitine deficiency (CUD), medium chain acyl-CoA dehydrogenase deficiency (MCAD), long chain 3 hydroxyacyl-CoA dehydrogenase deficiency (LCHAD), very long chain acyl-CoA dehydrogenase deficiency (VLCAD), or trifunctional protein deficiency (TFP).

In some embodiments, the fatty acid disorder is short chain acyl-CoA dehydrogenase (SCAD) deficiency, 3-hydroxyacyl-CoA dehydrogenase deficiency (M/SCHAD), glutaric acidemia type II (GA2), carnitine palmitoyltransferase I deficiency (CPT IA), carnitine palmitoyltransferase II deficiency (CPT II), or carnitine-acylcarnitine translocase (CACT).

In some embodiments, a genomic sample from a newborn is screened according to the methods provided herein for a gene variant associated with a fatty acid disorder. In some embodiments, a genomic sample from a newborn is screened according to the methods provided herein for a gene variant in at least one target region of at least one gene selected from the group consisting of SLC22A5, ACADM, ACADVL, HADHA, and HADHB. In some embodiments, a genomic sample from a newborn is screened according to the methods provided herein for a gene variant in at least one target region of at least one gene selected from the group consisting of ACADS, HADH, ETFA, ETFB, ETFDH, DECR1, CPT1A, CPT2, and SLC25A20. In some embodiments, the methods provided further include a diagnostic test for a fatty acid disorder. In some embodiments, the methods further include a diagnostic test for a fatty acid disorder if a gene variant is detected in at least one target region of at least one gene selected from the group consisting of SLC22A5, ACADM, ACADVL, HADHA, and HADHB. In some embodiments, the methods further include a diagnostic test for a fatty acid disorder if a gene variant is detected in at least one target region of at least one gene selected from the group consisting of ACADS, HADH, ETFA, ETFB, ETFDH, DECR1, CPT1A, CPT2, and SLC25A20. In some embodiments, the diagnostic test includes an acylcarnitine profile analysis of a biological sample from the newborn.

Medium Chain Acyl-CoA Dehydrogenase Deficiency (MCAD)

Medium-chain acyl-CoA dehydrogenase (MCAD) deficiency is a condition that prevents the body from converting certain fats to energy. Signs and symptoms typically appear during infancy or early childhood and include vomiting, lack of energy, and low blood sugar, seizures, breathing difficulties, liver problems, brain damage, coma, or sudden death. MCAD is the most common of the fatty acid oxidation conditions. Present screening methods include assays for detecting elevated levels of hexanoylcarnitine (C6), octanoylcarnitine (C8), decanoyl (C10), and/or C8/10.

In some embodiments, a genomic sample from a newborn is screened according to the methods provided herein for at least one gene variant in at least one target region of the acyl-CoA dehydrogenase, C-4 to C-12 straight chain (ACADM) gene at chromosome 1p31. In some embodiments, the methods provided further include a diagnostic test for MCAD. In some embodiments, the methods further include a diagnostic test for MCAD if at least one gene variant in at least one target region of ACADM is detected. In some embodiments, the diagnostic test includes an acylcarnitine profile analysis of a biological sample from the newborn. In some embodiments, the diagnostic test includes analysis of hexanoylcarnitine (C6), octanoylcarnitine (C8), decanoyl (C10), and/or C8/10 levels in a biological sample from the newborn

Long Chain 3 Hydroxyacyl-CoA Dehydrogenase Deficiency (LCHAD)

Long chain 3-hydroxyacyl-CoA dehydrogenase (LCHAD) deficiency prevents the body from converting certain fats to energy. Symptoms include feeding difficulties, lethargy, low blood sugar, weak muscle tone, retinal abnormalities, muscle pain or breakdown of muscle tissue and loss of sensation in arms and legs. Present screening methods include assays for detecting elevated levels of tetradecenolycarnitine (C14:1), hexadecanoylcarnitine (C16), 3-hydroxyhexadecanoylcarnitine (C16OH), octadecanoylcarnitine (C18), and/or 3-hydroxyoctadecanoylcarnitine (C18OH).

In some embodiments, a genomic sample from a newborn is screened according to the methods provided herein for at least one gene variant in at least one target region of the hydroxyacyl-CoA dehydrogenase/3-ketoacyl-CoA thiolase/enoyl-CoA hydratase (trifunctional protein), alpha subunit (HADHA) gene in chromosome 2p23 and/or hydroxyacyl-CoA dehydrogenase/3-ketoacyl-CoA thiolase/enoyl-CoA hydratase (trifunctional protein), beta subunit (HADHB) at chromosome 2p23. In some embodiments, the methods provided further include a diagnostic test for LCHAD. In some embodiments, the methods further include a diagnostic test for LCHAD if at least one gene variant in at least one target region of HADHA and/or HADHB is detected. In some embodiments, the diagnostic test includes an acylcarnitine profile analysis of a biological sample from the newborn. In some embodiments, the diagnostic test includes analysis of tetradecenoylcarnitine (C14:1), hexadecanoylcarnitine (C16), 3-hydroxyhexadecanoylcarnitine (C16OH), octadecanoylcarnitine (C18), and/or 3-hydroxyoctadecanoylcarnitine (C18OH) levels in a biological sample from the newborn.

Very Long Chain Acyl-CoA Dehydrogenase Deficiency (VLCAD)

Very long-chain acyl-CoA dehydrogenase (VLCAD) deficiency is a disorder in which the body is unable to convert very long-chain fatty acids into energy. Characteristic signs and symptoms of this disorder include lack of energy, and low blood sugar. Very long-chain fatty acids or partially metabolized fatty acids may also build up in tissues and damage the heart, liver, and muscles. Present screening tests include assays for elevated tetradecanolycarnitine (C14), tetradecenolycarnitine (C14:1), hexadecanoylcarnitine (C16), hexadecenoylcarnitine (C16:1), octadecanoylcarnitine (C18), and/or octadecenoylcarnitine (C18:1) levels.

In some embodiments, a genomic sample from a newborn is screened according to the methods provided herein for at least one gene variant in at least one target region of the acyl-CoA dehydrogenase, very long chain (ACADVL) gene in chromosome 17p13.1. In some embodiments, the methods provided further include a diagnostic test for VLCAD. In some embodiments, the methods further include a diagnostic test for VLCAD if at least one gene variant in at least one target region of ACADVL is detected. In some embodiments, the diagnostic test includes an acylcarnitine profile analysis of a biological sample from the newborn. In some embodiments, the diagnostic test includes analysis of tetradecanolycarnitine (C14), tetradecenolycarnitine (C14:1), hexadecanoylcarnitine (C16), hexadecenoylcarnitine (C16:1), octadecanoylcarnitine (C18), and/or octadecenoylcarnitine (C18:1) levels in a biological sample from the newborn.

Amino Acid Disorders

Amino acid disorders result from enzyme deficiencies involved in the catabolism any of a number of amino acids. Amino acid disorders are those conditions that lead to an abnormal buildup of particular amino acids. Present screening tests for amino acid disorders are MS/MS detection of particular amino acids. Currently a diagnosis is confirmed with quantitative amino acids, enzyme assay and/or mutation analysis.

In some embodiments, the amino acid disorder is arginosuccinic aciduria (ASA), citrullinemia (CIT) type I, maple syrup urine disease (MSUD), homocystinuria (HCY), phenylketonuria (PKU), or tyrosinemia (TYR I, II, III).

In some embodiments, the amino acid disorder is arginase deficiency (ARG), citrullinemia type II (CIT II), hypermethioninemia (MET), or disorders of biopterin regeneration.

In some embodiments, a genomic sample from a newborn is screened according to the methods provided herein for a gene variant associated with an amino acid disorder. In some embodiments, a genomic sample from a newborn is screened according to the methods provided herein for a gene variant in at least one target region of at least one gene selected from the group consisting of ASL, ASS1, BCKDHA, BCKDHB, DBT, DLD, CBS, MTHFR, MTR, MTRR, MMADHC, PAH, and FAH. In some embodiments, a genomic sample from a newborn is screened according to the methods provided herein for a gene variant in at least one target region of at least one gene selected from the group consisting of ARG1, SLC25A13, AHCY, GNMT, MAT1A, PAH, GCH1, PCBD1, PTS, QDPR, TAT, and HPD. In some embodiments, the methods provided further include a diagnostic test for an amino acid disorder. In some embodiments, the methods further include a diagnostic test for an amino acid disorder if a gene variant is detected in at least one target region of at least one gene selected from the group consisting of ASL, ASS1, BCKDHA, BCKDHB, DBT, DLD, CBS, MTHFR, MTR, MTRR, MMADHC, PAH, and FAH. In some embodiments, the methods further include a diagnostic test for an amino acid disorder if a gene variant is detected in at least one target region of at least one gene selected from the group consisting of ARGL SLC25A13, AHCY, GNMT, MAT1A, PAH, GCH1, PCBD1, PTS, QDPR, TAT, and HPD. In some embodiments, the diagnostic test includes an amino acid profile analysis of a biological sample from the newborn. In some embodiments, the diagnostic test includes an assay of the affected amino acid in a biological sample from the newborn. In some embodiments, the diagnostic test includes an analysis of leucine, methionine, and/or tyrosine.

Maple Syrup Urine Disease (MSUD)

Maple syrup urine disease is an inherited disorder in which the body is unable to process certain protein building blocks (amino acids) properly. Symptoms commonly begin in early infancy and include urine of a distinctive sweet odor, poor feeding, vomiting, lack of energy and developmental delay. If untreated, maple syrup urine disease can lead to seizures, coma, and death. This disorder may also be caused by mutations in the BCKDHB, DBT, and DLD genes. Maple syrup urine disease is currently detected by an elevation of the amino acid leucine and an abnormal leucine/alanine ratio.

In some embodiments, a genomic sample from a newborn is screened according to the methods provided herein for at least one gene variant in at least one target region of the branched chain keto acid dehydrogenase E1, alpha polypeptide (BCKDHA) gene in chromosome 19q13.1-q13.2; the branched chain keto acid dehydrogenase E1, beta polypeptide (BCKDHB) gene in chromosome 6q14.1; the dihydrolipoamide branched chain transacylase E2 (DBT) gene in chromosome 1p31; and/or the dihydrolipoamide dehydrogenase (DLD) gene at chromosome 7q31-q32. In some embodiments, the methods provided further include a diagnostic test for MSUD. In some embodiments, the methods further include a diagnostic test for MSUD if at least one gene variant in at least one target region of BCKDHA, BCKDHB, DBT, and/or DLD is detected. In some embodiments, the diagnostic test includes analysis of leucine levels in a biological sample from the newborn. In some embodiments, the diagnostic test includes analysis of leucine/alanine ratio in a biological sample from the newborn.

Propionic Acidemia (PA)

Propionic acidemia is an inherited disorder in which the body is unable to process certain parts of proteins and lipids (fats) properly. Mutations in the PCCA or PCCB genes prevent the production of functional propionyl-CoA carboxylase or reduce the enzyme's activity. The altered or missing enzyme prevents certain parts of proteins and lipids from being broken down properly. As a result, propionyl-CoA and other potentially toxic compounds can build up to toxic levels in the body. Within the first few days of life initial symptoms may arise including poor feeding, vomiting, loss of appetite, weak muscle tone (hypotonia), and lack of energy (lethargy). These symptoms sometimes progress to more serious medical problems, including heart abnormalities, seizures, coma, and possibly death. This condition can also be caused by mutation in the PCCA gene or the PCCB gene. PA is currently detected by measuring elevated levels of propionylcamitine (C3) and/or propionylcarnitine/acetylcarnitine (C3/C2).

In some embodiments, a genomic sample from a newborn is screened according to the methods provided herein for at least one gene variant in at least one target region of the propionyl CoA carboxylase, alpha polypeptide (PCCA) gene at chromosome 13q32 or a mutation in the propionyl CoA carboxylase, beta polypeptide (PCCB) gene at chromosome 3q21-q22. In some embodiments, the methods provided further include a diagnostic test for PA. In some embodiments, the methods further include a diagnostic test for PA if at least one gene variant in at least one target region of PCCA and/or PCCB is detected. In some embodiments, the diagnostic test includes analysis of propionylcamitine (C3) and/or propionylcarnitine/acetylcarnitine (C3/C2) levels in a biological sample from the newborn.

Argininosuccinate Lyase Deficiency (ASA)

Argininosuccinic aciduria is an inherited disorder that causes ammonia to accumulate in the blood. Ammonia, which is formed when proteins are broken down in the body, is toxic if the levels become too high. Argininosuccinic aciduria belongs to a class of genetic diseases called urea cycle disorders. The urea cycle is a sequence of reactions that occur in liver cells. It processes excess nitrogen, generated when protein is used by the body, to make a compound called urea that is excreted by the kidneys. In argininosuccinic aciduria, the enzyme that starts a specific reaction within the urea cycle is damaged or missing. The urea cycle cannot proceed normally, and nitrogen accumulates in the bloodstream in the form of ammonia. Mutations in the ASL gene cause argininosuccinic aciduria. Argininosuccinic aciduria usually becomes evident in the first few days of life. Symptoms include lack of energy, unwilling to eat, poorly controlled respiratory rate or body temperature, seizures, and coma. Currently, ASA is detected by measuring elevated levels of argininosuccinic acid/citrulline.

In some embodiments, a genomic sample from a newborn is screened according to the methods provided herein for at least one gene variant in at least one target region of the argininosuccinate lyase (ASL) gene at chromosome 7q11.21. In some embodiments, the methods provided further include a diagnostic test for ASA. In some embodiments, the methods further include a diagnostic test for ASA if at least one gene variant in at least one target region of ASL is detected. In some embodiments, the diagnostic test includes analysis of argininosuccinic acid/citrulline levels in a biological sample from the newborn.

Endocrine Disorders

Endocrine disorders are diseases related to the endocrine glands of the body. The endocrine system produces hormones, which are secreted into the bloodstream and affect other organs within the body to regulate processes, such as appetite, breathing, growth, fluid balance, feminization and virilization, and weight control. In some embodiments, the endocrine disorders are congenital adrenal hyperplasia (CAH) or congenital hypothyroidism (CH).

In some embodiments, a genomic sample from a newborn is screened according to the methods provided herein for a gene variant associated with an endocrine disorder. In some embodiments, a genomic sample from a newborn is screened according to the methods provided herein for a gene variant in at least one target region of at least one gene selected from the group consisting of DUOX2, PAX8, SLC5A5, TG, TPO, TSHB, TSHR, and CYP21A2. In some embodiments, the methods provided further include a diagnostic test for an endocrine disorder.

In some embodiments, the methods further include a diagnostic test for an endocrine disorder if a gene variant is detected in at least one target region of at least one gene selected from the group consisting of DUOX2, PAX8, SLC5A5, TG, TPO, TSHB, TSHR, and CYP21A2. In some embodiments, the diagnostic test includes a test for congenital hypothyroidism (CH). In some embodiments, the diagnostic test includes a test for congenital adrenal hyperplasia (CAH). In some embodiments, the diagnostic test includes an assay of thyroid hormones, T4 and TSH, in a biological sample from the newborn. In some embodiments, the diagnostic test includes an analysis 17-OH-progesterone in a biological sample from the newborn.

Congenital Adrenal Hyperplasia (CAH)

CAH is an inherited defect of cortisol synthesis, in which the adrenal gland cannot make cortisol and overproduces male hormones. Without cortisol, infants are at risk of death due to adrenal crisis and inability to regulate salt and fluids. The most common disorder is 21-hydroxylase deficiency. is an inherited disorder that affects the adrenal glands. Three types of 21-hydroxylase deficiency include the salt-wasting, simple virilizing, and non-classic types.

Infants may be symptomatic at birth, due to diminished cortisol production during gestation, which stimulates the fetal pituitary gland to produce ACTH resulting in excessive adrenal androgens. The androgens virilize female external genitalia, but ovaries and uterus are unaffected. Male infants may have increased scrotal pigmentation or may be asymptomatic. In 75 percent of cases, the 21-hydroxylase deficiency causes reduced production of mineralocorticoids. This reduction leads to a hypotensive, hyperkalemic, salt-losing crisis with rapid onset of adrenocortical failure within 7-28 days of birth, which can be fatal. In 25 percent of cases, the infant has a “non-salt losing” or “simple virilizing form.”

Currently screening is based on an immunoassay for a precursor steroid, 17-hydroxyprogesterone (17-OHP). Affected infants have high levels of 17-OHP, Infants with milder disorders have intermediate levels. Chromosome analysis is used to confirm gender if genitalia are ambiguous.

In some embodiments, a genomic sample from a newborn is screened according to the methods provided herein for at least one gene variant in at least one target region of the cytochrome P450, family 21, subfamily A, polypeptide 2 (CYP21A2) gene at chromosome 6p21.3. In some embodiments, the methods provided further include a diagnostic test for CAH. In some embodiments, the methods further include a diagnostic test for CAH if at least one gene variant in at least one target region of CYP2.1A2 is detected. In some embodiments, the diagnostic test includes analysis of 17-OH-progesterone levels in a biological sample from the newborn.

Hemoglobin Disorders

Hemoglobin disorders are those disorders that affect the production of function of hemoglobin. In some embodiments the hemoglobin disorder is sickle cell disease, metheglobinemia (beta-globin type), or beta thalassemia (thalassemia major and thalassemia intermedia).

In some embodiments the hemoglobin disorder is alpha thalassemia (hemoglobin disorder-Var-Hb).

In some embodiments, a genomic sample from a newborn is screened according to the methods provided herein for a gene variant associated with a hemoglobin disorder. In some embodiments, a genomic sample from a newborn is screened according to the methods provided herein for a gene variant in at least one target region of HBB. In some embodiments, the methods provided further include a diagnostic test for a hemoglobin disorder. In some embodiments, a genomic sample from a newborn is screened according to the methods provided herein for a gene variant in at least one target region of at least one gene selected from the group consisting of HBA1 and HBA2. In some embodiments, the methods further include a diagnostic test for a hemoglobin disorder if a gene variant is detected in at least one target region of HBB. In some embodiments, the methods further include a diagnostic test for a hemoglobin disorder if a gene variant is detected in at least one target region of at least one gene selected from the group consisting of HBA1 and HBA2. In some embodiments, the diagnostic test includes a test for sickle cell disease, metheglobinemia (beta-globin type), or beta thalassemia (thalassemia major and thalassemia intermedia), In some embodiments, the diagnostic test includes an assay for hemoglobinopathies using isoelectric focusing (IEF) of a biological sample from the newborn. In some embodiments, the diagnostic test includes an assay for hemoglobinopathies using high performance liquid chromatography (HPLC) of a biological sample from the newborn. In some embodiments, the diagnostic test includes an assay for hemoglobinopathies using both IEF and HPLC of a biological sample from the newborn.

Other Disorders

Other disorders include those conditions which are genetic disorders that fall outside of the categories of disorders listed above. In some embodiments, the other condition is biotinidase deficiency (BIOT), cystic fibrosis (CF), galactosemia type I, hearing loss ((1) nonsyndromic deafness, (2) palmoplantar karatoderma, (3) hystrix-like ichthyosis, (4) Bart-Pumphrey syndrome, (5) Vohwinkel syndrome, (6) karatitis-ichthyosis-deafness (KID), (7) erythrokeratodermia variabilis et progressive (EKVP), (8) Clouston syndrome), severe combined immunodeficiency (SCID), or X-linked severe combined immunodeficiency (SCID).

In some embodiments, the other disorder is galactosemia type III or galactosemia type II.

In other embodiments, the other disorder is X-linked adrenoleukodystrophy, adrenomyeloneuropathy Addison disease (X-ALD), 2,4 dienoyl-CoA reductase deficiency, Pompe disease (GAA deficiency), Krabbe disease, Gaucher disease (types I, II, & III), Fabry disease, mucopolysaccharidosis type I (MPS I), congenital disorder of deglycosylation type 1v, Niemann-Pick disease (type C1), or Niemann-Pick disease (type C2).

In some embodiments, a genomic sample from a newborn is screened according to the methods provided herein for a gene variant in at least one target region of at least one gene selected from the group consisting of BTD, CFTR, GALT, GJB2, GJB3, GJB6, ADA, and IL2RG. In some embodiments, the methods further include a diagnostic test for the associated disorder if a gene variant is detected in at least one target region of at least one gene selected from the group consisting of BTD, CFTR, GALT, GJB2, GJB3, GJB6, ADA, and IL2RG. In some embodiments, the diagnostic test includes a test for biotinidase deficiency (BIOT), cystic fibrosis (CF), galactosemia type I, hearing loss, severe combined immunodeficiency (SCID), or X-linked severe combined immunodeficiency (SCID). In some embodiments, the diagnostic test includes an assay for immunotrypsinogen, biotinidase, and/or GALT enzyme activity.

Galactosemia Type I

Galactosemia is a disorder that prevents the body from processing a simple sugar called galactose into energy. Galactosemia type I is the most common and most severe form of the disorder. If infants are not promptly treated complications can arise within the first few days of life. Complications include feeding difficulties, lack of energy, failure to thrive, jaundice, liver damage and bleeding. Mutations in the GALT gene are responsible for classic galactosemia (type I). Most of these genetic changes almost completely eliminate the activity of the enzyme produced from the GALT gene, preventing the normal processing of galactose and resulting in the life-threatening signs and symptoms of this disorder. Another GALT gene mutation, known as the Duarte variant, reduces but does not eliminate the activity of the enzyme. People with the Duarte variant tend to have much milder features of galactosemia. Currently, two screening tests are used to detect galactosemia in a two-tiered sequence, the GALT activity test and the galactose (Hill) test. The GALT enzyme activity test depends upon fluorescence produced by the normal galactose enzyme cascade in red blood cells. It does not differentiate milder variants from severe defects. All infants are screened with the GALT test. The Hill test is a fluorometric chemical spot test that measures galactose and galactose-1-phosphate. Galactose metabolites are greatly elevated in infants with galactosemia if they are receiving a lactose-containing formula or breast milk. All infants with an abnormal GALT or who have been transfused are screened with the Hill test.

In some embodiments, a genomic sample from a newborn is screened according to the methods provided herein for at least one gene variant in at least one target region of the galactose-1-phosphate uridylyltransferase (GALT) gene at chromosome 9p13. In some embodiments, the methods provided further include a diagnostic test for Galactosemia. In some embodiments, the methods further include a diagnostic test for galactosemia if at least one gene variant in at least one target region of GALT is detected. In some embodiments, the diagnostic test includes analysis of GALT enzyme activity level and/or galactose and galactose-1-phosphate levels in a biological sample from the newborn. In some embodiments, a biological sample from the newborn is analyzed using a CALF enzyme activity test and/or a Hill test. In some embodiments, the method further includes treatment of the disorder using the standard treatment for galactosemia, if a gene variant in GALT is identified.

Kits and Articles of Manufacture

In some embodiments, there are provided compositions comprising a collection of oligonucleotide primers for selective amplification of plurality of target regions of each of PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8. In some embodiments, the composition further includes oligonucleotide primers for target regions of additional genes listed in Table 2.

In some embodiments, there are provided kits comprising a collection of oligonucleotide primers for sequencing of each of PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8. In some embodiments, the kit further includes oligonucleotide primers for target regions of additional genes listed in Table 2. In some embodiments, the kit further comprises one or more reagents for performing a sequencing reaction.

TABLE 1
Gene and Diseases Reference: Genes, Cytogenetic Location, Disease, Brief Description, Signs and Symptoms

| | | | | |
|---|---|---|---|---|
| | Cytogenic | | | |
| Gene | Location | Disease | Brief Description | Signs & Symptoms |

Core Conditions

| | | | | | |
|---|---|---|---|---|---|
| Organic | PCCA | 13q32 | Propionic acidemia | Propionic acidemia is an | Within the first few days of life initial symptoms |
| Acid | | | (PROP) | inherited disorder in which | may arise including poor feeding, vomiting, loss of |
| Disorders | | | | the body is unable to | appetite, weak muscle tone (hypotonia), and lack |
| | | | | process certain parts of | of energy (lethargy). These symptoms sometimes |
| | | | | proteins and lipids (fats) | progress to more serious medical problems, |
| | | | | properly. | including heart abnormalities, seizures, coma, and |
| | | | | | possibly death. This condition can also be caused |
| | | | | | by mutation in the PCCB gene. |
| | PCCB | 3q21-q22 | Propionic acidemia | Propionic acidemia is an | Within the first few days of life initial symptoms |
| | | | (PROP) | inherited disorder in which | may arise including poor feeding, vomiting, loss of |
| | | | | the body is unable to | appetite, weak muscle tone (hypotonia), and lack |
| | | | | process certain parts of | of energy (lethargy). These symptoms sometimes |
| | | | | proteins and lipids (fats) | progress to more serious medical problems, |
| | | | | properly. | including heart abnormalities, seizures, coma, and |
| | | | | | possibly death. This condition can also be caused |
| | | | | | by mutation in the PCCA gene. |
| | MCEE | 2p13.3 | Methylmalonic acidemia | Methylmalonic acidemia is | Effects of the disorder, which normally arise during |
| | | | (MUT) | an inherited disorder in | infancy, range from mild to life threatening. |
| | | | | which the body is unable to | Symptoms include vomiting, dehydration, weak |
| | | | | process certain proteins | muscle tone, developmental delay, lethargy, |
| | | | | and fats (lipids) properly. | enlarged liver, failure to thrive. Long-term |
| | | | | | complications can lead to intellectual disability, |
| | | | | | kidney disease, and pancreatitis and can lead to |
| | | | | | coma or death. This disorder may also arise due to |
| | | | | | mutations in the MUT, MMAA, MMAB, and |
| | | | | | MMADHC genes. |
| | MMAA | 4q31.21 | Methylmalonic acidemia | Methylmalonic acidemia is | Effects of the disorder, which normally arise during |
| | | | (MUT) | an inherited disorder in | infancy, range from mild to life threatening. |
| | | | | which the body is unable to | Symptoms include vomiting, dehydration, weak |
| | | | | process certain proteins | muscle tone, developmental delay, lethargy, |
| | | | | and fats (lipids) properly. | enlarged liver, failure to thrive. Long-term |
| | | | | | complications can lead to intellectual disability, |
| | | | | | kidney disease, and pancreatitis and can lead to |
| | | | | | coma or death. This disorder may also arise due to |
| | | | | | mutations ain the MCEE, MUT, MMAB, and |
| | | | | | MMADHC genes. |
| | MMAB | 12q24 | Methylmalonic acidemia | Methylmalonic acidemia is | Effects of the disorder, which normally arise during |
| | | | (MUT) | an inherited disorder in | infancy, range from mild to life threatening. |
| | | | | which the body is unable to | Symptoms include vomiting, dehydration, weak |
| | | | | process certain proteins | muscle tone, developmental delay, lethargy, |
| | | | | and fats (lipids) properly. | enlarged liver, failure to thrive. Long-term |
| | | | | | complications can lead to intellectual disability, |
| | | | | | kidney disease, and pancreatitis and can lead to |
| | | | | | coma or death. This disorder may also arise due to |
| | | | | | mutations in the MCEE, MUT, MMAA, and |
| | | | | | MMADHC genes. |
| | MMADHC | 2q23.2 | (I)Methylmalonic | (I) Methylmalonic acidemia | (I) Effects of the disorder, which normally arise |
| | | | acidemia (MUT) | is an inherited disorder in | during infancy, range from mild to life threatening. |
| | | | (II)Homocystinuria (HCY) | which the body is unable to | Symptoms include vomiting, dehydration, weak |
| | | | | process certain proteins | muscle tone, developmental delay, lethargy, |
| | | | | and fats (lipids) properly. | enlarged liver, failure to thrive. Long-term |
| | | | | (II) Homocystinuria is an | complications can lead to intellectual disability, |
| | | | | inherited disorder in which | kidney disease, and pancreatitis and can lead to |
| | | | | the body is unable to | coma or death. This disorder may also arise due to |
| | | | | process certain building | mutations in the MCEE, MUT, MMAA, and |
| | | | | blocks of proteins (amino | MMAB genes. |
| | | | | acids) properly. | (II) Multiple forms of this disorder exist but most |
| | | | | | common symptoms include nearsightedness, |
| | | | | | dislocation of the lens at the front of the eye, |
| | | | | | increased risk of abnormal blood clotting, and |
| | | | | | brittle bones. |
| | | | | | Although most often mutations in the CBS gene |
| | | | | | cause this disorder, it may also arise from |
| | | | | | mutations in the CBS, MTHFR, MTR, and MTRR. |
| | MUT | 6p12.3 | Methylmalonic acidemia | Methylmalonic acidemia is | Effects of the disorder, which normally arise during |
| | | | (MUT) | an inherited disorder in | infancy, range from mild to life threatening. |
| | | | | which the body is unable to | Symptoms include vomiting, dehydration, weak |
| | | | | process certain proteins | muscle tone, developmental delay, lethargy, |
| | | | | and fats (lipids) properly. | enlarged liver, failure to thrive. Long-term |
| | | | | | complications can lead to intellectual disability, |
| | | | | | kidney disease, and pancreatitis and can lead to |
| | | | | | coma or death. This disorder may also arise due to |
| | | | | | mutations in the MCEE, MMAA, MMAB, and |
| | | | | | MMADHC genes. |
| | IVD | 15q14-q15 | Isovaleric acidemia | Isovaleric acidemia is a | Cases vary from mild to life threatening and in |
| | | | (IVA) | rare disorder in which the | severe cases the features of the disorder become |
| | | | | body is unable to process | apparent within days after birth. Symptoms include |
| | | | | certain proteins properly | poor feeding, vomiting, seizures, lethargy, coma |
| | | | | | and possibly death. An odor of sweaty feet is |
| | | | | | present with acute illness. |
| | MCCC1 | 3q27 | 3-methylcrotonyl-CoA | 3-methylcrotonyl-CoA | Affected infants often appear normal at birth but |
| | | | carboxylase deficiency | carboxylase deficiency is | develop signs and symptoms in infancy or early |
| | | | (3-MCC) | an inherited disorder in | childhood. Symptoms include vomiting and |
| | | | | which the body is unable to | diarrhea, lethargy, weak muscle tone, delayed |
| | | | | process certain proteins | development, seizures and coma. Many problems |
| | | | | properly. | can be prevented with early detection. This |
| | | | | | disorder may also be caused my mutations in the |
| | | | | | MCCC2 gene. |
| | MCCC2 | 5q12-q13 | 3-methylcrotonyl-CoA | 3-methylcrotonyl-CoA | Affected infants often appear normal at birth but |
| | | | carboxylase deficiency | carboxylase deficiency is | develop signs and symptoms in infancy or early |
| | | | (3-MCC) | an inherited disorder in | childhood. Symptoms include vomiting and |
| | | | | which the body is unable to | diarrhea, lethargy, weak muscle tone, delayed |
| | | | | process certain proteins | development, seizures and coma. Many problems |
| | | | | properly. | can be prevented with early detection. This |
| | | | | | disorder may also be caused my mutations in the |
| | | | | | MCCC1 gene. |
| | HMGCL | 1p36.1- | 3-hydroxy-3- | 3-hydroxy-3- | Characteristics of this disorder generally arise |
| | | p35 | methylglutaryl-CoA lyase | methylglutaryl-CoA lyase | within the first year of life. Signs and symptoms |
| | | | deficiency | deficiency (also known as | include vomiting, diarrhea, dehydration, lethargy, |
| | | | (HMG) | HMG-CoA lyase | weak muscle tone, hypoglycemia. Can lead to |
| | | | | deficiency) is an | breathing problems, convulsions, coma and death. |
| | | | | uncommon inherited | Commonly mistaken for Reye syndrome. |
| | | | | disorder in which the body | |
| | | | | cannot process a particular | |
| | | | | protein building block | |
| | | | | (amino acid) called | |
| | | | | leucine. | |
| | HLCS | 21q22.13 | Multiple Carboxylase | Another name for MCD is | The signs and symptoms of this disorder generally |
| | | | Deficiency (MCD) | holocarboxylase | appear within the first few months of life. They |
| | | | | synthetase deficiency, a | include breathing problems, skin rash, hair loss, |
| | | | | disorder where the body is | lethargy. Biotin supplements may prevent |
| | | | | unable to use the vitamin | complications. |
| | | | | biotin effectively. | |
| | ACAT1 | 11q22.3 | Beta-ketothiolase | Beta-ketothiolase | Signs and symptoms typically appear between the |
| | | | deficiency (βKT) | deficiency is an inherited | ages of six to 24 months. Episodes called |
| | | | | disorder in which the body | ketoacidotic attacks may occur causing symptoms |
| | | | | cannot effectively process | such as vomiting, dehydration, difficulty breathing, |
| | | | | a protein building block | extreme lethargy, and occasionally seizures. |
| | | | | (amino acid) called | Infections, fasting, or increased intake of protein |
| | | | | isoleucine. | rich foods frequently triggers these ketoacidotic |
| | | | | | attacks. Attacks can also lead to coma. |
| | GCDH | 19p13.2 | Glutaric acidemia type I | Glutaric acidemia type I is | Most often signs and symptoms first occur in |
| | | | (GA1) | an inherited disorder in | infancy and early childhood. Symptoms vary from |
| | | | | which the body is unable to | mild to severe and may result in poor motor control |
| | | | | process certain proteins | and intellectual disability. Some individuals develop |
| | | | | properly. | bleeding in the brain or eyes. |
| Fatty Acid | SLC22A5 | 5q23.3 | Primary carnitine | Primary carnitine | Signs and symptoms typically appear during |
| Disorders | | | deficiency (CUD) | deficiency is a condition | infancy or early childhood and can include severe |
| | | | | that prevents the body | brain dysfunction, a weakened and enlarged heart, |
| | | | | from using certain fats. | confusion, vomiting, muscle weakness, and low |
| | | | | | blood sugar, heart failure, liver problems, and |
| | | | | | sudden death. |
| | ACADM | 1p31 | Medium-chain acyl-CoA | Medium-chain acyl-CoA | Signs and symptoms typically appear during |
| | | | dehydrogenase (MCAD) | dehydrogenase (MCAD) | infancy or early childhood and can include |
| | | | deficiency | deficiency is a condition | vomiting, lack of energy, and low blood sugar, |
| | | | | that prevents the body | seizures, breathing difficulties, liver problems, brain |
| | | | | from converting certain fats | damage, coma, and sudden death. |
| | | | | to energy. | |
| | ACADVL | 17p13.1 | Very long-chain acyl-CoA | Very long-chain acyl-CoA | Characteristic signs and symptoms of this disorder |
| | | | dehydrogenase (VLCAD) | dehydrogenase (VLCAD) | include lack of energy, and low blood sugar. Very |
| | | | deficiency | deficiency is a disorder in | long-chain fatty acids or partially metabolized fatty |
| | | | | which the body is unable to | acids may also build up in tissues and damage the |
| | | | | convert very long-chain | heart, liver, and muscles. |
| | | | | fatty acids into energy. | |
| | HADHA | 2p23 | Trifunctional protein | (I) Long chain 3- | (I) Symptoms include feeding difficulties, lethargy, |
| | | | deficiency (TFP) | hydroxyacyl-CoA | low blood sugar, weak muscle tone, retinal |
| | | | | dehydrogenase | abnormalities. May experience muscle pain or |
| | | | | (LCHAD) deficiency | breakdown of muscle tissue and loss of sensation |
| | | | | prevents the body | in arms and legs. |
| | | | | from converting | (II) Symptoms include feeding difficulties, lethargy, |
| | | | | certain fats to energy. | low blood sugar, and liver problems. High risk for |
| | | | | (II) Mitochondrial | heart problems, breathing difficulty, coma and |
| | | | | trifunctional protein | sudden death. This disorder can also arise from |
| | | | | deficiency prevents the | mutations in the HADHB gene. |
| | | | | body from converting | |
| | | | | certain fats into energy. | |
| | HADHB | 2p23 | Trifunctional protein | Mitochondrial trifunctional | Symptoms include feeding difficulties, lethargy, low |
| | | | deficiency (TFP) | protein deficiency prevents | blood sugar, and liver problems. High risk for heart |
| | | | | the body from converting | problems, breathing difficulty, coma and sudden |
| | | | | certain fats into energy. | death. This disorder can also arise from mutations |
| | | | | | in the HADHA gene. |
| Amino | ASL | 7q11.21 | Argininosuccinic aciduria | Argininosuccinic aciduria is | Argininosuccinic aciduria usually becomes evident |
| Acid | | | (ASA) | an inherited disorder that | in the first few days of life. Symptoms include lack |
| Disorders | | | | causes ammonia to | of energy, unwilling to eat, poorly controlled |
| | | | | accumulate in the blood. | respiratory rate or body temperature, seizures, and |
| | | | | | coma. |
| | ASS1 | 9q34.1 | Citrullinemia (CIT) | Citrullinemia is an inherited | Type I (classic citrullinemia): usually becomes |
| | | | type I | disorder that causes | evident in the first few days of life. Symptoms |
| | | | | ammonia and other toxic | include lack of energy, poor feeding, vomiting, |
| | | | | substances to accumulate | seizures, and loss of consciousness later in |
| | | | | in the blood. | childhood or adulthood. This later-onset form is |
| | | | | | associated with intense headaches, partial loss of |
| | | | | | vision, problems with balance and muscle |
| | | | | | coordination (ataxia), and lethargy. |
| | | | | | Type II is caused by mutations in the SLC25A13 |
| | | | | | gene. |
| | BCKDHA | 19q13.1- | Maple Syrup Urine | Maple syrup urine disease | Symptoms commonly begin in early infancy and |
| | | q13.2 | Disease (MSUD) | is an inherited disorder in | include urine of a distinctive sweet odor, poor |
| | | | | which the body is unable to | feeding, vomiting, lack of energy and |
| | | | | process certain protein | developmental delay. If untreated, maple syrup |
| | | | | building blocks (amino | urine disease can lead to seizures, coma, and |
| | | | | acids) properly. | death. This disorder may also be caused by |
| | | | | | mutations in the BCKDHB, DBT, and DLD genes. |
| | BCKDHB | 6q14.1 | Maple Syrup Urine | Maple syrup urine disease | Symptoms commonly begin in early infancy and |
| | | | Disease (MSUD) | is an inherited disorder in | include urine of a distinctive sweet odor, poor |
| | | | | which the body is unable to | feeding, vomiting, lack of energy and |
| | | | | process certain protein | developmental delay. If untreated, maple syrup |
| | | | | building blocks (amino | urine disease can lead to seizures, coma, and |
| | | | | acids) properly. | death. This disorder may also be caused by |
| | | | | | mutations in the BCKDHA, DBT, and DLD genes. |
| | DBT | 1p31 | Maple Syrup Urine | Maple syrup urine disease | Symptoms commonly begin in early infancy and |
| | | | Disease (MSUD) | is an inherited disorder in | include urine of a distinctive sweet odor, poor |
| | | | | which the body is unable to | feeding, vomiting, lack of energy and |
| | | | | process certain protein | developmental delay. If untreated, maple syrup |
| | | | | building blocks (amino | urine disease can lead to seizures, coma, and |
| | | | | acids) properly. | death. This disorder may also be caused by |
| | | | | | mutations in the BCKDHA, BCKDHB, and DLD |
| | | | | | genes. |
| | DLD | 7q31-q32 | Maple Syrup Urine | Maple syrup urine disease | Symptoms commonly begin in early infancy and |
| | | | Disease (MSUD) | is an inherited disorder in | include urine of a distinctive sweet odor, poor |
| | | | | which the body is unable to | feeding, vomiting, lack of energy and |
| | | | | process certain protein | developmental delay. If untreated, maple syrup |
| | | | | building blocks (amino | urine disease can lead to seizures, coma, and |
| | | | | acids) properly. | death. This disorder may also be caused by |
| | | | | | mutations in the BCKDHA, BCKDHB, and DBT |
| | | | | | genes. |
| | CBS | 21q22.3 | Homocystinuria (HCY) | Homocystinuria is an | Multiple forms of this disorder exist but most |
| | | | | inherited disorder in which | common symptoms include nearsightedness, |
| | | | | the body is unable to | dislocation of the lens at the front of the eye, |
| | | | | process certain building | increased risk of abnormal blood clotting, and |
| | | | | blocks of proteins (amino | brittle bones. |
| | | | | acids) properly. | Although most often mutations in the CBS gene |
| | | | | | cause this disorder, it may also arise from |
| | | | | | mutations in the MTHFR, MTR, MTRR and |
| | | | | | MMADHC genes. |
| | MTHFR | 1p36.3 | Homocystinuria (HCY) | Homocystinuria is an | Multiple forms of this disorder exist but most |
| | | | | inherited disorder in which | common symptoms include nearsightedness, |
| | | | | the body is unable to | dislocation of the lens at the front of the eye, |
| | | | | process certain building | increased risk of abnormal blood clotting, and |
| | | | | blocks of proteins (amino | brittle bones. |
| | | | | acids) properly. | Although most often mutations in the CBS gene |
| | | | | | cause this disorder, it may also arise from |
| | | | | | mutations in the CBS, MTR, MTRR and MMADHC |
| | | | | | genes. |
| | MTR | 1q43 | Homocystinuria (HCY) | Homocystinuria is an | Multiple forms of this disorder exist but most |
| | | | | inherited disorder in which | common symptoms include nearsightedness, |
| | | | | the body is unable to | dislocation of the lens at the front of the eye, |
| | | | | process certain building | increased risk of abnormal blood clotting, and |
| | | | | blocks of proteins (amino | brittle bones. |
| | | | | acids) properly. | Although most often mutations in the CBS gene |
| | | | | | cause this disorder, it may also arise from |
| | | | | | mutations in the CBS, MTHFR, MTRR and |
| | | | | | MMADHC genes. |
| | MTRR | 5p15.31 | Homocystinuria (HCY) | Homocystinuria is an | Multiple forms of this disorder exist but most |
| | | | | inherited disorder in which | common symptoms include nearsightedness, |
| | | | | the body is unable to | dislocation of the lens at the front of the eye, |
| | | | | process certain building | increased risk of abnormal blood clotting, and |
| | | | | blocks of proteins (amino | brittle bones. |
| | | | | acids) properly. | Although most often mutations in the CBS gene |
| | | | | | cause this disorder, it may also arise from |
| | | | | | mutations in the CBS, MTHFR, MTR, and |
| | | | | | MMADHC genes. |
| | PAH | 12q22- | Phenylketonuria | Phenylketonuria is an | Classic PKU presents normally until the infant is a |
| | | q24.2 | (PKU) | inherited disorder that | few months old. Without treatment, these children |
| | | | | increases the levels of a | develop permanent intellectual disability. Seizures, |
| | | | | substance called | delayed development, behavioral problems, and |
| | | | | phenylalanine in the blood. | psychiatric disorders are also common. Other, less |
| | | | | | severe forms have a smaller risk of brain damage. |
| | | | | | Untreated individuals may have a musty or mouse- |
| | | | | | like odor as a side effect of excess phenylalanine |
| | | | | | in the body. |
| | FAH | 15q25.1 | Tyrosinemia (TYR I, II, III) | Tyrosinemia is a genetic | Type 1: Symptoms include failure to thrive, |
| | | | | disorder characterized by | diarrhea, vomiting, jaundice, cabbage odor, and |
| | | | | elevated blood levels of | nosebleeds. Can lead to liver and kidney failure |
| | | | | the amino acid tyrosine. | and impact the nervous system. |
| | | | | | Type II: Symptoms include excessive tearing, |
| | | | | | sensitivity to light, eye pain, redness, and |
| | | | | | intellectual disability. |
| | | | | | Type III: Symptoms include Intellectual disability, |
| | | | | | seizures, and loss of balance. |
| | | | | | Mutations in the HPD, and TAT genes may also |
| | | | | | cause tyrosinemia. |
| Endocrine | DUOX2 | 15q15.3 | Congenital hypothyroidism | Congenital hypothyroidism | If untreated, congenital hypothyroidism can lead to |
| Disorders | | | (CH) | is a condition that affects | intellectual disability and abnormal growth. |
| | | | | infants from birth | Other genes that may cause congenital |
| | | | | (congenital) and results | hypothyroidism include the PAX8, SLC5A5, TG, |
| | | | | from a partial or complete | TPO, TSHB, and TSHR genes. |
| | | | | loss of thyroid function | |
| | | | | (hypothyroidism). | |
| | PAX8 | 2q13 | Congenital hypothyroidism | Congenital hypothyroidism | If untreated, congenital hypothyroidism can lead to |
| | | | (CH) | is a condition that affects | intellectual disability and abnormal growth. |
| | | | | infants from birth | Other genes that may cause congenital |
| | | | | (congenital) and results | hypothyroidism include the DUOX2, SLC5A5, TG, |
| | | | | from a partial or complete | TPO, TSHB, and TSHR genes. |
| | | | | loss of thyroid function | |
| | | | | (hypothyroidism). | |
| | SLC5A5 | 19p13.11 | Congenital hypothyroidism | Congenital hypothyroidism | If untreated, congenital hypothyroidism can lead to |
| | | | (CH) | is a condition that affects | intellectual disability and abnormal growth. |
| | | | | infants from birth | Other genes that may cause congenital |
| | | | | (congenital) and results | hypothyroidism include the DUOX2, PAX8, TG, |
| | | | | from a partial or complete | TPO, TSHB, and TSHR genes. |
| | | | | loss of thyroid function | |
| | | | | (hypothyroidism). | |
| | TG | 8q24 | Congenital hypothyroidism | Congenital hypothyroidism | If untreated, congenital hypothyroidism can lead to |
| | | | (CH) | is a condition that affects | intellectual disability and abnormal growth. |
| | | | | infants from birth | Other genes that may cause congenital |
| | | | | (congenital) and results | hypothyroidism include the DUOX2, PAX8, |
| | | | | from a partial or complete | SLC5A5, TPO, TSHB, and TSHR genes. |
| | | | | loss of thyroid function | |
| | | | | (hypothyroidism). | |
| | TPO | 2p25 | Congenital hypothyroidism | Congenital hypothyroidism | If untreated, congenital hypothyroidism can lead to |
| | | | (CH) | is a condition that affects | intellectual disability and abnormal growth. |
| | | | | infants from birth | Other genes that may cause congenital |
| | | | | (congenital) and results | hypothyroidism include the DUOX2, PAX8, |
| | | | | from a partial or complete | SLC5A5, TG, TSHB, and TSHR genes. |
| | | | | loss of thyroid function | |
| | | | | (hypothyroidism). | |
| | TSHB | 1p13 | Congenital hypothyroidism | Congenital hypothyroidism | If untreated, congenital hypothyroidism can lead to |
| | | | (CH) | is a condition that affects | intellectual disability and abnormal growth. |
| | | | | infants from birth | Other genes that may cause congenital |
| | | | | (congenital) and results | hypothyroidism include the DUOX2, PAX8, |
| | | | | from a partial or complete | SLC5A5, TG, TPO, and TSHR genes. |
| | | | | loss of thyroid function | |
| | | | | (hypothyroidism). | |
| | TSHR | 14q31 | Congenital hypothyroidism | Congenital hypothyroidism | If untreated, congenital hypothyroidism can lead to |
| | | | (CH) | is a condition that affects | intellectual disability and abnormal growth. |
| | | | | infants from birth | Other genes that may cause congenital |
| | | | | (congenital) and results | hypothyroidism include the DUOX2, PAX8, |
| | | | | from a partial or complete | SLC5A5, TG, TPO, and TSHB genes. |
| | | | | loss of thyroid function | |
| | | | | (hypothyroidism). | |
| | CYP21A2 | 6p21.3 | 21-hydroxylase deficiency | 21-hydroxylase deficiency | Signs and symptoms for the salt-wasting type |
| | | | (CAH) | is an inherited disorder that | include low hormone production, loss of sodium in |
| | | | | affects the adrenal glands. | urine, poor feeding, weight loss, dehydration, and |
| | | | | Three types of this | vomiting. Females with the salt-wasting and simple |
| | | | | disorder include the salt- | virilizing forms typically have external genitalia that |
| | | | | wasting, simple virilizing, | do not look clearly female or male. Individuals with |
| | | | | and non-classic types. | these forms may also experience decreased |
| | | | | | fertility and other hormone related symptoms. |
| Hemoglobin | HBB | 11p15.5 | (I ) Sickle cell disease | All three disorders affect | Symptoms of the different disorders are as follows: |
| Disorders | | | (II)Metheglobinemia, beta- | the production or function | (1) Anemia, repeated infections, periodic episodes |
| | | | globin type | of hemoglobin. | of pain. |
| | | | (III) Beta thalassemia: | | (2) Bluish appearance of the skin, mucous |
| | | | thalassemia major and | | membranes, or area under the fingernails. |
| | | | thalassemia intermedia | | (3) Anemia, pale skin, weakness, fatigue, risk of |
| | | | | | abnormal blood clots, enlarged spleen, liver, and |
| | | | | | heart. |
| Other | BTD | 3p25 | Biotinidase deficiency | Biotinidase deficiency is an | The signs and symptoms of biotinidase deficiency |
| Disorders | | | (BIOT) | inherited disorder in which | typically appear within the first few months of life, |
| | | | | the body is unable to reuse | but the age of onset varies. Symptoms often |
| | | | | and recycle the vitamin | include seizures, muscle weakness, breathing |
| | | | | biotin. | problems, and delayed development. If left |
| | | | | | untreated, the disorder can lead to hearing loss, |
| | | | | | eye abnormalities and loss of vision, problems with |
| | | | | | movement and balance, skin rashes, hair loss, and |
| | | | | | a fungal infection called candidiasis. Immediate |
| | | | | | treatment and lifelong management with biotin |
| | | | | | supplements can prevent many of these |
| | | | | | complications. |
| | CFTR | 7q31.2 | Cystic fibrosis (CF) | Cystic fibrosis is an | The disorder's most common signs and symptoms |
| | | | | inherited disease | include progressive damage to the respiratory |
| | | | | characterized by the | system and chronic digestive system problems. |
| | | | | buildup of thick, sticky | Problems with digestion can lead to diarrhea, |
| | | | | mucus that can damage | malnutrition, poor growth, and weight loss. In |
| | | | | many of the body's organs. | adolescence or adulthood, a shortage of insulin |
| | | | | | can cause a form of diabetes known as cystic |
| | | | | | fibrosis-related diabetes mellitus (CFRDM). |
| | GALT | 9p13 | Galactosemia type I | Galactosemia is a disorder | Type I: This is the most common and most severe |
| | | | | that prevents the body | form of the disorder. If infants are not promptly |
| | | | | from processing a simple | treated complications can arise within the first few |
| | | | | sugar called galactose into | days of life. Complications include feeding |
| | | | | energy. | difficulties, lack of energy, failure to thrive, |
| | | | | | jaundice, liver damage and bleeding. |
| | | | | | Type II and III are caused by mutations in the |
| | | | | | GALE and GALT genes. |
| | GJB2 | 13q11-q12 | Hearing Loss | Several conditions can | (1) Damage to inner ear structure resulting in |
| | | | (1) Nonsyndromic | cause hearing damage or | permanent hearing loss. (2) Mild to profound |
| | | | deafness | loss in infants. Listed are | hearing loss, skin abnormalities. Increased risk of |
| | | | (2) Palmoplantar | the disorders caused by | skin cancer. |
| | | | karatoderma | mutations in the GJB2 | (3) |
| | | | (3) Hystrix-like ichthyosis | gene. | Dry, thick scaly skin and profound hearing loss, |
| | | | (4) Bart-Pumphrey | | which may worsen over time. Increased risk of |
| | | | syndrome | | skin cancer. |
| | | | (5) Vohwinkel syndrome | | (4) |
| | | | (6) Karatitis-ichthyosis- | | Discoloration of nails and wart-like growths on |
| | | | deafness (KID) | | fingers and toes. Hearing loss is profound. |
| | | | | | (5) Symptoms include hearing loss, starfish- |
| | | | | | shaped patches of skin on fingers, toes and knees, |
| | | | | | and tight fibrous tissue around fingers and toes |
| | | | | | resulting in amputation. |
| | | | | | (6) Profound hearing loss, vision loss |
| | GJB3 | 1p34 | Hearing Loss | Several conditions can | (1) Damage to inner ear structure resulting in |
| | | | (1)Nonsyndromic deafness | cause hearing damage or | permanent hearing loss. |
| | | | (2)Erythrokeratodermia | loss in infants. Listed are | (2) Symptoms include rough, thickened reddish- |
| | | | variabilis et progressive | the disorders caused by | brown patches and patches of reddened skin. |
| | | | (EKVP) | mutations in the GJB3 | |
| | | | | gene | |
| | GJB6 | 13q12 | Hearing Loss | Several conditions can | (1) Damage to inner ear structure |
| | | | (1) Nonsyndromic | cause hearing damage or | resulting in permanent hearing loss. |
| | | | deafness | loss in infants. Listed are | (2) Symptoms include sparse, patchy, fragile scalp |
| | | | (2) Clouston syndrome | the disorders caused by | hair and abnormal fingernails and toenails. |
| | | | | mutations in the GJB3 | |
| | | | | gene | |
| | ADA | 20q13.12 | Severe combined | Adenosine deaminase | The main symptoms arise due to a very weakened |
| | | | immunodeficiency (SCID) | (ADA) deficiency is an | immune system and commonly include |
| | | | | inherited disorder that | pneumonia, chronic diarrhea, and widespread skin |
| | | | | damages the immune | rashes. Affected children tend to grow slower than |
| | | | | system and causes severe | healthy children and some experience |
| | | | | combined | developmental delay. |
| | | | | immunodeficiency (SCID). | |
| | IL2RG | Xq13.1 | X-linked severe combined | X-linked severe combined | Individuals with this disorder are prone to infection, |
| | | | immunodeficiency (SCID) | immunodeficiency (SCID) | which may cause life-threatening illnesses. May |
| | | | | is an inherited disorder of | experience chronic diarrhea or skin rashes. |
| | | | | the immune system that | |
| | | | | occurs almost exclusively | |
| | | | | in males. | |

Secondary Conditions

| | | | | | |
|---|---|---|---|---|---|
| Organic | MLYCD | 16q24 | Malonyl CoA | Malonyl CoA | Symptoms appear in early childhood and result in |
| Acid | | | decarboxylase deficiency | decarboxylase deficiency | delayed development, weak muscle tone, seizures, |
| Disorders | | | (MAL) | is a condition that prevents | diarrhea, vomiting, low blood sugar, and |
| | | | | the body from converting | cardiomyopathy. |
| | | | | certain fats to energy. | |
| | ACAD8 | 11q25 | Isobutyryl-CoA | Isobutyryl-CoA | Most effected individuals do not experience |
| | | | dehydrogenase (IBD) | dehydrogenase (IBD) | symptoms. A few children with IBD deficiency have |
| | | | deficiency | deficiency is a condition | developed features such as a weakened and |
| | | | | that disrupts the | enlarged heart, weak muscle tone, developmental |
| | | | | breakdown of certain | delay, and anemia. |
| | | | | proteins. | |
| | ACADSB | 10q26.13 | 2-methylbutyryl-CoA | 2-methylbutyryl-CoA | The initial symptoms often include poor feeding, |
| | | | dehydrogenase deficiency | dehydrogenase deficiency | lack of energy, vomiting, and an irritable mood. |
| | | | | is a type of organic acid | These symptoms sometimes progress to serious |
| | | | | disorder in which the body | medical problems such as difficulty breathing, |
| | | | | is unable to process | seizures, and coma. Additional problems can |
| | | | | proteins properly | include poor growth, vision problems, learning |
| | | | | | disabilities, muscle weakness, and delays in motor |
| | | | | | skills such as standing and walking. |
| | AUH | 9q22.31 | 3-methylglutaconic | The name 3- | There are five types of 3-methylglutaconic aciduria |
| | | | aciduria (3MGA) type I | methylglutaconic aciduria | numbered I, II, III, IV and V. |
| | | | | is used to describe five | Type I: Mutations in the AUH gene cause Type I 3- |
| | | | | different disorders that | methylglutaconic aciduria. Symptoms include |
| | | | | impair the functioning of | speech delay, delay in the development of mental |
| | | | | energy-producing centers | and motor skills, elevated levels of acid in the |
| | | | | within cells (mitochondria). | blood, abnormal muscle tone, and spasms and |
| | | | | | weakness of the arms and legs |
| | | | | | Types II, III, IV and IV are caused by mutations in |
| | | | | | the DNAJC19, OPA3, and TAZ genes. |
| | DNAJC19 | 3q26.33 | 3-methylglutaconic | The name 3- | There are five types of 3-methylglutaconic aciduria |
| | | | aciduria (3MGA) type V | methylglutaconic aciduria | numbered I, II, III, IV and V. |
| | | | | is used to describe five | Type V: Mutations in the DNAJC19 gene cause |
| | | | | different disorders that | Type V 3-methylglutaconic aciduria. Symptoms |
| | | | | impair the functioning of | include an enlarged and weakened heart, inability |
| | | | | energy-producing centers | to control voluntary muscle movements, growth |
| | | | | within cells (mitochondria). | failure, mild intellectual disability and optic atrophy |
| | | | | | Types I, II, III, and V are caused by mutations in |
| | | | | | the AUH, OPA3, and TAZ genes. |
| | OPA3 | 19q13.32 | 3-methylglutaconic | The name 3- | There are five types of 3-methylglutaconic aciduria |
| | | | aciduria (3MGA) type I | methylglutaconic aciduria | numbered I, II, III, IV and V. |
| | | | | is used to describe five | Type III (Costeff Optic Syndrome): Mutations in |
| | | | | different disorders that | the OPA3 gene cause the Type III form of this |
| | | | | impair the functioning of | disorder which is characterized by degeneration of |
| | | | | energy-producing centers | the optic nerves, inability to maintain posture, poor |
| | | | | within cells (mitochondria). | muscle tone, gradual increase in involuntary |
| | | | | | jerking movements, and general decrease in |
| | | | | | cognitive function. |
| | | | | | Types I, II, IV and IV are caused by mutations in |
| | | | | | the AUH, DNAJC19, and TAZ genes. |
| | TAZ | Xq28 | 3-methylglutaconic | The name 3- | There are five types of 3-methylglutaconic aciduria |
| | | | aciduria (3MGA) type I | methylglutaconic aciduria | numbered I, II, III, IV and V. |
| | | | | is used to describe five | Type II (Barth Syndrome): Mutations in the TAZ |
| | | | | different disorders that | gene cause the Type II form of this disorder, which |
| | | | | impair the functioning of | is characterized by, and enlarge and weakened |
| | | | | energy-producing centers | heart, reoccurring infections, muscle weakness, |
| | | | | within cells (mitochondria). | and muscle spasms. |
| | | | | | Types I, III, IV and IV are caused by mutations in |
| | | | | | the AUH, DNAJC19, and OPA3 genes. |
| | HSD17B10 | Xp11.2 | 3-hydroxy-2-methylbutyryl- | Another name for | This disorder is more severe in males. Normal |
| | | | CoA dehydrogenase | 2M3HBA is | early development, but experience developmental |
| | | | deficiency (2M3HBA) | 17β-hydroxysteroid | regression around age 5 resulting in intellectual |
| | | | | dehydrogenase type 10 | disability and loss of motor skills. Hearing and |
| | | | | (HSD10) deficiency. | vision loss may also occur. |
| Fatty Acid | ACADS | 12q24.31 | Short-chain acyl-CoA | Short-chain acyl-CoA | Signs and symptoms can include vomiting, low |
| Disorders | | | dehydrogenase (SCAD) | dehydrogenase (SCAD) | blood sugar (hypoglycemia), a lack of energy, poor |
| | | | deficiency | deficiency is a condition | feeding, failure to gain weight and grow at the |
| | | | | that prevents the body | expected rate, poor muscle tone, seizures, |
| | | | | from converting certain fats | developmental delay, and a small head size. |
| | | | | into energy | |
| | HADH | 4q22-q26 | 3-hydroxyacyl-CoA | 3-hydroxyacyl-CoA | Characteristics of this disorder typically arise |
| | | | dehydrogenase deficiency | dehydrogenase deficiency | during infancy or early childhood. Signs and |
| | | | (M/SCHAD) | is an inherited condition | symptoms include muscle weakness, poor |
| | | | | that prevents the body | appetite, vomiting, diarrhea, lethargy, liver |
| | | | | from converting certain fats | problems, low blood sugar, seizures, life- |
| | | | | to energy, particularly | threatening heart and breaking problems, coma, |
| | | | | during prolonged periods | and sudden death (SIDS). |
| | | | | without food (fasting). | |
| | ETFA | 15q23-q25 | Glutaric acidemia type II | Glutaric acidemia type II is | Due to buildup of incompletely processed proteins |
| | | | (GA2) | an inherited disorder that | and fats symptoms can arise including weakness, |
| | | | | interferes with the body's | poor feeding, decreased activity, and vomiting. |
| | | | | ability to break down | Other abnormalities include brain malformations, |
| | | | | proteins and fats to | an enlarged liver, a weakened and enlarged heart, |
| | | | | produce energy. | fluid-filled cysts and other malformations of the |
| | | | | | kidneys, unusual facial features, and genital |
| | | | | | abnormalities. |
| | | | | | Mutations in the ETFB and ETFDH genes can also |
| | | | | | cause this disorder. |
| | ETFB | 19q13.3 | Glutaric acidemia type II | Glutaric acidemia type II is | Due to buildup of incompletely processed proteins |
| | | | (GA2) | an inherited disorder that | and fats symptoms can arise including weakness, |
| | | | | interferes with the body's | poor feeding, decreased activity, and vomiting. |
| | | | | ability to break down | Other abnormalities include brain malformations, |
| | | | | proteins and fats to | an enlarged liver, a weakened and enlarged heart, |
| | | | | produce energy. | fluid-filled cysts and other malformations of the |
| | | | | | kidneys, unusual facial features, and genital |
| | | | | | abnormalities. |
| | | | | | Mutations in the ETFA and ETFDH genes can also |
| | | | | | cause this disorder. |
| | ETFDH | 4q32-q35 | Glutaric acidemia type II | Glutaric acidemia type II is | Due to buildup of incompletely processed proteins |
| | | | (GA2) | an inherited disorder that | and fats symptoms can arise including weakness, |
| | | | | interferes with the body's | poor feeding, decreased activity, and vomiting. |
| | | | | ability to break down | Other abnormalities include brain malformations, |
| | | | | proteins and fats to | an enlarged liver, a weakened and enlarged heart, |
| | | | | produce energy. | fluid-filled cysts and other malformations of the |
| | | | | | kidneys, unusual facial features, and genital |
| | | | | | abnormalities. |
| | | | | | Mutations in the ETFA and ETFB genes can also |
| | | | | | cause this disorder. |
| | CPT1A | 11q13.2 | Carnitine | Carnitine | Signs and symptoms of CPT I deficiency often |
| | | | palmitoyltransferase I | palmitoyltransferase I | appear during early childhood. Signs and |
| | | | deficiency (CPT IA) | (CPT I) deficiency is a | symptoms include low blood sugar and low ketone |
| | | | | condition that prevents the | levels, enlarged liver, liver malfunction, and |
| | | | | body from using certain | elevated levels of carnitine in the blood. Individuals |
| | | | | fats for energy, particularly | with CPT I deficiency are at risk for nervous |
| | | | | during periods without food | system damage, liver failure, seizures, coma, and |
| | | | | (fasting). | sudden death. |
| | CPT2 | 1p32 | Carnitine | Carnitine | Type I (Lethal Neonatal): Appears soon after birth |
| | | | palmitoyltransferase II | palmitoyltransferase II | with symptoms including respiratory failure, |
| | | | deficiency (CPT II) | (CPT II) deficiency is a | seizures, liver failure, various abnormal heart |
| | | | | condition that prevents the | conditions, and hypoketotic hypoglycemia. |
| | | | | body from using certain | Type II (Severe Infantile Hepatocardiomuscular): |
| | | | | fats for energy, particularly | Appears within the first year of life with symptoms |
| | | | | during periods without food | including hypoketotic hypoglycemia, seizures, |
| | | | | (fasting). | enlarged liver, and various abnormal heart |
| | | | | | conditions. |
| | | | | | Type II (Myopathic): Characterized by reoccurring |
| | | | | | muscle pain and weakness and discolored urine. |
| | SLC25A20 | 3p21.31 | Carnitine-acylcarnitine | Carnitine-acylcarnitine | Many infants with CACT deficiency do not survive |
| | | | translocase (CACT) | translocase (CACT) | the newborn period without treatment. Signs and |
| | | | | deficiency is a condition | symptoms include low blood sugar, low levels of |
| | | | | that prevents the body | ketones, excess ammonia in the blood, enlarged |
| | | | | from using certain fats for | liver, and weakened heart muscles. |
| | | | | energy, particularly during | |
| | | | | periods without food | |
| | | | | (fasting). | |
| Amino | ARG1 | 6q23 | Arginase deficiency (ARG) | Arginase deficiency (ARG) | Arginase deficiency usually becomes evident by |
| Acid | | | | is an inherited disorder that | about the age of 3. It most often appears as |
| Disorders | | | | causes the amino acid | stiffness, caused by abnormal tensing of the |
| | | | | arginine (a building block | muscles, slow growth patterns, developmental |
| | | | | of proteins) and ammonia | delays, intellectual disabilities, seizures, tremors, |
| | | | | to accumulate gradually in | and difficulty with coordination. |
| | | | | the blood. | |
| | SLC25A13 | 7q21.3 | Citrullinemia type II | Citrullinemia is an inherited | Type I: Caused by mutations in the ASS1 gene. |
| | | | (CIT II) | disorder that causes | Type II: Caused by mutations in the SLC25A13 |
| | | | | ammonia and other toxic | gene and chiefly affects the nervous system, |
| | | | | substances to accumulate | causing confusion, restlessness, memory loss, |
| | | | | in the blood. | abnormal behaviors (such as aggression, |
| | | | | | irritability, and hyperactivity), seizures, and coma. |
| | AHCY | 20q11.22 | Hypermethioninemia | Hypermethioninemia is an | People with hypermethioninemia often do not show |
| | | | (MET) | excess of a particular | any symptoms. Some individuals with |
| | | | | protein building block | hypermethioninemia exhibit intellectual disability |
| | | | | (amino acid), called | and other neurological problems; delays in motor |
| | | | | methionine, in the blood. | skills such as standing or walking; sluggishness; |
| | | | | | muscle weakness; liver problems; unusual facial |
| | | | | | features; and their breath, sweat, or urine may |
| | | | | | have a smell resembling boiled cabbage. This |
| | | | | | disorder may also be caused by mutations in the |
| | | | | | GNMT and MAT1A genes. |
| | GNMT | 6p12 | Hypermethioninemia | Hypermethioninemia is an | People with hypermethioninemia often do not show |
| | | | (MET) | excess of a particular | any symptoms. Some individuals with |
| | | | | protein building block | hypermethioninemia exhibit intellectual disability |
| | | | | (amino acid), called | and other neurological problems; delays in motor |
| | | | | methionine, in the blood. | skills such as standing or walking; sluggishness; |
| | | | | | muscle weakness; liver problems; unusual facial |
| | | | | | features; and their breath, sweat, or urine may |
| | | | | | have a smell resembling boiled cabbage. This |
| | | | | | disorder may also be caused by mutations in the |
| | | | | | AHCY and MAT1A genes. |
| | MAT1A | 10q22 | Hypermethioninemia | Hypermethioninemia is an | People with hypermethioninemia often do not show |
| | | | (MET) | excess of a particular | any symptoms. Some individuals with |
| | | | | protein building block | hypermethioninemia exhibit intellectual disability |
| | | | | (amino acid), called | and other neurological problems; delays in motor |
| | | | | methionine, in the blood. | skills such as standing or walking; sluggishness; |
| | | | | | muscle weakness; liver problems; unusual facial |
| | | | | | features; and their breath, sweat, or urine may |
| | | | | | have a smell resembling boiled cabbage. This |
| | | | | | disorder may also be caused by mutations in the |
| | | | | | AHCY and GNMT genes. |
| | GCH1 | 14q22.1- | Disorders of biopterin | Tetrahydrobiopterin | Infants with this deficiency often appear normal at |
| | | q22.2 | regeneration | deficiency is a rare | birth but over time begin to exhibit mild to severe |
| | | | | disorder characterized by a | symptoms and signs such as intellectual disability, |
| | | | | shortage of a molecule | developmental problems, movement disorders, |
| | | | | called tetrahydrobiopterin, | difficulty swallowing, seizures, behavioral |
| | | | | which leads to an | problems, and inability to control body |
| | | | | increased level of | temperature. |
| | | | | phenylalanine in the body. | |
| | PCBD1 | 10q22 | Disorders of biopterin | Tetrahydrobiopterin | Infants with this deficiency often appear normal at |
| | | | regeneration | deficiency is a rare | birth but over time begin to exhibit mild to severe |
| | | | | disorder characterized by a | symptoms and signs such as intellectual disability, |
| | | | | shortage of a molecule | developmental problems, movement disorders, |
| | | | | called tetrahydrobiopterin, | difficulty swallowing, seizures, behavioral |
| | | | | which leads to an | problems, and inability to control body |
| | | | | increased level of | temperature. |
| | | | | phenylalanine in the body. | |
| | PTS | 11q22.3 | Disorders of biopterin | Tetrahydrobiopterin | Infants with this deficiency often appear normal at |
| | | | regeneration | deficiency is a rare | birth but over time begin to exhibit mild to severe |
| | | | | disorder characterized by a | symptoms and signs such as intellectual disability, |
| | | | | shortage of a molecule | developmental problems, movement disorders, |
| | | | | called tetrahydrobiopterin, | difficulty swallowing, seizures, behavioral |
| | | | | which leads to an | problems, and inability to control body |
| | | | | increased level of | temperature. |
| | | | | phenylalanine in the body. | |
| | QDPR | 4p15.31 | Disorders of biopterin | Tetrahydrobiopterin | Infants with this deficiency often appear normal at |
| | | | regeneration | deficiency is a rare | birth but over time begin to exhibit mild to severe |
| | | | | disorder characterized by a | symptoms and signs such as intellectual disability, |
| | | | | shortage of a molecule | developmental problems, movement disorders, |
| | | | | called tetrahydrobiopterin, | difficulty swallowing, seizures, behavioral |
| | | | | which leads to an | problems, and inability to control body |
| | | | | increased level of | temperature. |
| | | | | phenylalanine in the body. | |
| | TAT | 16q22.1 | Tyrosinemia (TYR I, II, III) | Tyrosinemia is a genetic | Type 1: Symptoms include failure to thrive, |
| | | | | disorder characterized by | diarrhea, vomiting, jaundice, cabbage odor, and |
| | | | | elevated blood levels of | nosebleeds. Can lead to liver and kidney failure |
| | | | | the amino acid tyrosine. | and impact the nervous system. |
| | | | | | Type II: Symptoms include excessive tearing, |
| | | | | | sensitivity to light, eye pain, redness, and |
| | | | | | intellectual disability. |
| | | | | | Type III: Symptoms include Intellectual disability, |
| | | | | | seizures, and loss of balance. |
| | | | | | Mutations in the FAH and HPD genes may also |
| | | | | | cause tyrosinemia. |
| | HPD | 12q24.31 | Tyrosinemia (TYR I, II, III) | Tyrosinemia is a genetic | Type 1: Symptoms include failure to thrive, |
| | | | | disorder characterized by | diarrhea, vomiting, jaundice, cabbage odor, and |
| | | | | elevated blood levels of | nosebleeds. Can lead to liver and kidney failure |
| | | | | the amino acid tyrosine. | and impact the nervous system. |
| | | | | | Type II: Symptoms include excessive tearing, |
| | | | | | sensitivity to light, eye pain, redness, and |
| | | | | | intellectual disability. |
| | | | | | Type III: Symptoms include Intellectual disability, |
| | | | | | seizures, and loss of balance. |
| | | | | | Mutations in the FAH and TAT genes may also |
| | | | | | cause tyrosinemia. |
| Hemoglobin | HBA1 | 16p13.3 | Alpha thalassemia | Alpha thalassemia is a | This disorder causes a shortage of red blood cells |
| Disorders | | | (Hemoglobin Disorder-Var- | blood disorder that | (anemia), which can cause pale skin, weakness, |
| | | | Hb) | reduces the production of | fatigue, along with other complications. |
| | | | | hemoglobin. | Two types have been observed: |
| | | | | | (I) Hb Bart: characterized by severe anemia, |
| | | | | | enlarged liver and spleen, heart defects, |
| | | | | | abnormalities of urinary system or genitalia. |
| | | | | | (II) HbH: Characterized by mild to moderate |
| | | | | | anemia, jaundice, overgrowth of jaw or prominent |
| | | | | | forehead. |
| | | | | | This disorder can also be caused by mutations in |
| | | | | | the HBA2 gene. |
| | HBA2 | 16p13.3 | Alpha thalassemia | Alpha thalassemia is a | This disorder causes a shortage of red blood cells |
| | | | (Hemoglobin Disorder-Var- | blood disorder that | (anemia), which can cause pale skin, weakness, |
| | | | Hb) | reduces the production of | fatigue, along with other complications. |
| | | | | hemoglobin. | Two types have been observed: |
| | | | | | (I) Hb Bart: characterized by severe anemia, |
| | | | | | enlarged liver and spleen, heart defects, |
| | | | | | abnormalities of urinary system or genitalia. |
| | | | | | (II) HbH: Characterized by mild to moderate |
| | | | | | anemia, jaundice, overgrowth of jaw or prominent |
| | | | | | forehead. |
| | | | | | This disorder can also be caused by mutations in |
| | | | | | the HBA1 gene. |
| Other | GALE | 1p36-p35 | Galactosemia type III | Galactosemia is a disorder | Type III: Symptoms include cataracts, delayed |
| Disorders | | | | that prevents the body | growth and development, intellectual disability, |
| | | | | from processing a simple | liver disease, and kidney problems. |
| | | | | sugar called galactose into | Type I and II are caused by mutations in the |
| | | | | energy. | GALK1 and GALT genes. |
| | GALK1 | 17q24 | Galactosemia type II | Galactosemia is a disorder | Type II: Affected infants may develop cataracts, |
| | | | | that prevents the body | but otherwise experience few additional |
| | | | | from processing a simple | complications. |
| | | | | sugar called galactose into | Type I and III are caused by mutations in the GALE |
| | | | | energy. | and GALT genes. |

Additional Conditions

| | | | | |
|---|---|---|---|---|
| ABCD1 | Xq28 | X-linked | X-linked | Cerebral form (childhood): learning and behavioral |
| | | adrenoleukodystrophy | adrenoleukodystrophy | problems, aggressive behavior, vision problems, |
| | | Adrenomyeloneuropathy | Adrenomyeloneuropathy is | difficulty swallowing, poor coordination, and |
| | | Addison disease | a genetic disorder, | impaired adrenal function. |
| | | (X-ALD) | effecting primarily males | Adrenomyeloneuropathy form (early adulthood to |
| | | | that negatively impacts the | middle age): stiffness and weakness in the legs, |
| | | | nervous system and the | urinary and genital tract disorders and changes in |
| | | | adrenal glands. | behavior and thinking ability. |
| | | | | Addison disease: adrenocortical insufficiency only. |
| DECR1 | 8q21.3 | 2,4 Dienoyl-CoA reductase | 2,4 Dienoyl-CoA reductase | Poor appetite, vomiting, irritability, weak muscle |
| | | deficiency | deficiency is a condition | tone, short torso, small body, and small head size. |
| | | | associated with respiratory | |
| | | | acidosis in infancy. | |
| GAA | 17q25.2- | Pompe disease | Pompe disease is an | Type I (Classic): Onset begins within a few months |
| | 25.3 | (GAA deficiency) | inherited disorder caused | and triggers symptoms such as muscle weakness, |
| | | | by the buildup of a | enlarged liver, and heart defects. If untreated, this |
| | | | complex sugar called | form leads to death within the first year of life. |
| | | | glycogen in the body's | Type II (Non-classic): Typically appears by the age |
| | | | cells causing an inability to | of one with characteristics including delayed motor |
| | | | function properly. | skills and progressive muscle weakness. This form |
| | | | | can cause death within early childhood. |
| | | | | Type III (Late-onset): May not become apparent |
| | | | | until adulthood and may cause muscle weakness |
| | | | | and progressively worsening breathing problems. |
| GALC | 14q31 | Krabbe Disease | Krabbe disease (also | Symptoms usually arise before the age of one and |
| | | | called globoid cell | include irritability, muscle weakness, feeding |
| | | | leukodystrophy) is a | problems, fever, and slowed mental and physical |
| | | | degenerative disorder that | development, vision loss and seizures. |
| | | | affects the nervous | |
| | | | system. | |
| GBA | 1q21 | Gaucher disease | Gaucher disease is an | Type I (Non-neuronopathic): Characteristics |
| | | (types I, II, & III) | inherited disorder that | include enlarged liver and spleen, anemia, easy |
| | | | affects many of the body's | bruising caused by decreased platelets, lung |
| | | | organs and tissues. | disease and bone abnormalities. |
| | | | | Types II & III (Neuronopathic): Both forms have |
| | | | | similar symptoms to Type I but additionally affect |
| | | | | the central nervous system. Additional conditions |
| | | | | may include abnormal eye movements, seizures |
| | | | | and brain damage. |
| | | | | Perinatal Lethal: The most severe form that |
| | | | | causes life-threatening complications including |
| | | | | extensive swelling, skin abnormalities, and |
| | | | | neurological complications. |
| | | | | Cardiovascular: Causes hardening of heart valves, |
| | | | | eye abnormalities, and bone diseases. |
| GLA | Xq22 | Fabry disease | Fabry disease is an | With disorder characters beginning in childhood, |
| | | | inherited disorder that | complications may include life-threatening kidney |
| | | | results from the buildup of | damage, heart attack and stroke. Symptoms |
| | | | a particular type of fat, | include pain in hands and feet, dark red spots on |
| | | | called | skin, decreased ability to sweat, corneal opacity, |
| | | | globotriaosylceramide, in | ear ringing, gastrointestinal problems. |
| | | | the body's cells. | |
| IDUA | 4p16.3 | Mucopolysaccharidosis | Mucopolysaccharidosis | Typically no signs and symptoms are present at |
| | | type I | type I (MPS I) is a | birth. Other characteristics of the disorder that |
| | | (MPS I) | condition that affects many | slowly progress include large head, fluid in the |
| | | | parts of the body. | bread, heart abnormalities, large tongue, upper |
| | | | | respiratory infection, clouding of cornea resulting in |
| | | | | vision loss, and hearing loss. Severely affected |
| | | | | individuals eventually lose basic motor skills. |
| NGLY1 | 3p24.2 | Congenital Disorder of | Congenital disorder of | Features include seizures, abnormal eye |
| | | Deglycosylation type 1v | deglysosylation is an | movements, liver dysfunction, and microcephaly. |
| | | | autosomal recessive | |
| | | | multisystem disorder | |
| | | | caused by a defect in | |
| | | | glycoprotein synthesis. | |
| NPC1 | 18q11.2 | Niemann-Pick disease | Niemann-Pick disease is | Niemann-Pick disease is divided into four main |
| | | (type C1) | an inherited condition | types: A, B, C1 and C2. Mutations in the NPC1 |
| | | | involving lipid metabolism, | gene are responsible for type C1. Symptoms for |
| | | | which is the breakdown, | this form usually appear in childhood and often |
| | | | transport, and use of fats | include severe liver disease, breathing difficulties, |
| | | | and cholesterol in the | developmental delay, seizures, poor muscle tone, |
| | | | body. | lack of coordination, problems feeding, and inability |
| | | | | to move the eyes vertically. Type A and B are |
| | | | | caused by mutations in the SMPD1 gene and type |
| | | | | C2 is caused by mutations in the NPC2 gene. |
| NPC2 | 14q24.3 | Niemann-Pick disease | Niemann-Pick disease is | Niemann-Pick disease is divided into four main |
| | | (type C2) | an inherited condition | types: A, B, C1 and C2. Mutations in the NPC2 |
| | | | involving lipid metabolism, | gene are responsible for type C2. Symptoms for |
| | | | which is the breakdown, | this form usually appear in childhood and often |
| | | | transport, and use of fats | include severe liver disease, breathing difficulties, |
| | | | and cholesterol in the | developmental delay, seizures, poor muscle tone, |
| | | | body. | lack of coordination, problems feeding, and inability |
| | | | | to move the eyes vertically. Type A and B are |
| | | | | caused by mutations in the SMPD1 gene and type |
| | | | | C1 is caused by mutations in the NPC1 gene. |

TABLE 2
Exemplary Genes List showing the affected genes and core, secondary and additional conditions.

| | | Metabolic Disorder | | | | | | | | | | | | |
|---|---|---|---|---|---|---|---|---|---|---|---|---|---|---|
| | | | Fatty Acid | | Endocrine | Hemoglobin | | | | | | | | |
| | ACMG Code | Organic Acid | oxidation | Amino Acid | Disorder | Disorder | Other Disorder | Genes Affected | | | | | | |
| Core (or Primary) Conditions | PROP | X | | | | | | PCCA | PCCB | | | | | |
| | MUT | X | | | | | | MUT | MMAA | MMAB | MMADHC | MCEE | | |
| | Cbl A,B | X | | | | | | MUT | MMAA | MMAB | MMADHC | MCEE | | |
| | IVA | X | | | | | | IVD | | | | | | |
| | 3-MCC | X | | | | | | MCCC1 | MCCC2 | | | | | |
| | HMG | X | | | | | | HMGCL | | | | | | |
| | MCD | X | | | | | | HLCS | | | | | | |
| | βKT | X | | | | | | ACAT1 | | | | | | |
| | GA1 | X | | | | | | GCDH | | | | | | |
| | CUD | | X | | | | | SLC22A5 | | | | | | |
| | MCAD | | X | | | | | ACADM | | | | | | |
| | VLCAD | | X | | | | | ACADVL | | | | | | |
| | LCHAD | | X | | | | | HADHA | | | | | | |
| | TFP | | X | | | | | HADHA | HADHB | | | | | |
| | ASA | | | X | | | | ASL | | | | | | |
| | CIT | | | X | | | | ASS1 | | | | | | |
| | MSUD | | | X | | | | BCKDHA | BCKDHB | DBT | DLD | | | |
| | HCY | | | X | | | | CBS | MTHFR | MTR | MTRR | MMADHC | | |
| | PKU | | | X | | | | PAH | | | | | | |
| | TYR I | | | X | | | | FAH | | | | | | |
| | CH | | | | X | | | DUOX2 | PAX8 | SLC5A5 | TG | TPO | TSHB | TSHR |
| | CAH | | | | X | | | CYP21A2 | | | | | | |
| | Hb SS | | | | | X | | HBB | | | | | | |
| | Hb S/β TH | | | | | X | | HBB | | | | | | |
| | Hb S/C | | | | | X | | HBB | | | | | | |
| | BIOT | | | | | | X | BTD | | | | | | |
| | CF | | | | | | X | CFTR | | | | | | |
| | GALT | | | | | | X | GALT | | | | | | |
| | HEAR | | | | | | X | GJB2 | GJB3 | GJB6 | | | | |
| | SCID | | | | | | X | ADA | IL2RG | | | | | |
| Secondary Conditions | CBL C, D | X | | | | | | MMADHC | | | | | | |
| | MAL | X | | | | | | MLYCD | | | | | | |
| | IBD | X | | | | | | ACAD8 | | | | | | |
| | 2MBG | X | | | | | | ACADSB | | | | | | |
| | 3MGA | X | | | | | | AUH | DNAJC19 | OPA3 | TAZ | | | |
| | 2M3HBA | X | | | | | | HSD17810 | | | | | | |
| | SCAD | | X | | | | | ACADS | | | | | | |
| | M/SCHAD | | X | | | | | HADH | | | | | | |
| | GA2 | | X | | | | | ETFA | ETFB | ETFDH | | | | |
| | MCAT | | X | | | | | SCL25A20 | | | | | | |
| | DE RED | | X | | | | | DECR1 | | | | | | |
| | CPT IA | | X | | | | | CPT1A | | | | | | |
| | CPT II | | X | | | | | CPT2 | | | | | | |
| | CACT | | X | | | | | SLC25A20 | | | | | | |
| | ARG | | | X | | | | ARG1 | | | | | | |
| | CIT II | | | X | | | | SLC25A13 | | | | | | |
| | MET | | | X | | | | AHCY | GNMT | MAT1A | | | | |
| | H-PHE | | | X | | | | PAH | | | | | | |
| | BIOPT (BS) | | | X | | | | GCH1 | PCBD1 | PTS | QDPR | | | |
| | BIOPT (Reg) | | | X | | | | GCH1 | PCBD1 | PTS | QDPR | | | |
| | TYR II | | | X | | | | TAT | | | | | | |
| | TYR III | | | X | | | | HPD | | | | | | |
| | Var Hb | | | | | X | | HBA1 | HBA2 | HBB | | | | |
| | GALE | | | | | | X | GALE | | | | | | |
| | GALK | | | | | | X | GALK1 | | | | | | |
| ADDED Conditions | Krabbe | | | | | | | GALC | | | | | | |
| | Gaucher | | | | | | | GBA | | | | | | |
| | Niemann-Pick A/B | | | | | | | NPC1 | NPC2 | | | | | |
| | Pompe | | | | | | | GAA | | | | | | |
| | Fabry | | | | | | | GLA | | | | | | |
| | MPS I | | | | | | | IDUA | | | | | | |
| | X-ALD | | | | | | | ABCD1 | | | | | | |
| | | | | | | | | NGLY1 | | | | | | |

TABLE 3
Cytogenic Locations for genes included in Exemplary Genes List

| Gene | Cytogenic location |
|---|---|
| ACAD8 | 11q25 |
| ACADM | 1p31 |
| ACADS | 12q24.31 |
| ACADSB | 10q26.13 |
| ACADVL | 17p13.1 |
| ACAT1 | 11q22.3 |
| ADA | 20q13.12 |
| AHCY | 20q11.22 |
| ARG1 | 6q23 |
| ASL | 7q11.21 |
| ASS1 | 9q34.1 |
| AUH | 9q22.31 |
| BCKDHA | 19q13.1-q13.2 |
| BCKDHB | 6q14.1 |
| BTD | 3p25 |
| CBS | 21q22.3 |
| CFTR | 7q31.2 |
| CPT1A | 11q13.2 |
| CPT2 | 1p32 |
| CYP21A2 | 6p21.3 |
| DBT | 1p31 |
| DECR1 | 8q21.3 |
| DLD | 7q31-q32 |
| DNAJC19 | 3q26.33 |
| DUOX2 | 15q15.3 |
| ETFA | 15q23-q25 |
| ETFB | 19q13.3 |
| ETFDH | 4q32-q35 |
| FAH | 15q25.1 |
| GALE | 1p36-p35 |
| GALK1 | 17q24 |
| GALT | 9p13 |
| GCDH | 19p13.2 |
| GCH1 | 14q22.1-q22.2 |
| GNMT | 6p12 |
| HADH | 4q22-q26 |
| HADHA | 2p23 |
| HADHB | 2p23 |
| HBA1 | 16p13.3 |
| HBA2 | 16p13.3 |
| HBB | 11p15.5 |
| HLCS | 21q22.13 |
| HMGCL | 1p36.1-p35 |
| HPD | 12q24.31 |
| HSD17B10 | Xp11.2 |
| IL2RG | Xq13.1 |
| IVD | 15q14-q15 |
| MAT1A | 10q22 |
| MCCC1 | 3q27 |
| MCCC2 | 5q12-q13 |
| MCEE | 2p13.3 |
| MLYCD | 16q24 |
| MMAA | 4q31.21 |
| MMAB | 12q24 |
| MMADHC | 2q23.2 |
| MTHFR | 1p36.3 |
| MTR | 1q43 |
| MTRR | 5p15.31 |
| MUT | 6p12.3 |
| OPA3 | 19q13.32 |
| PAH | 12q22-q24.2 |
| PAX8 | 2q13 |
| PCBD1 | 10q22 |
| PCCA | 13q32 |
| PCCB | 3q21-q22 |
| PTS | 11q22.3 |
| QDPR | 4p15.31 |
| SLC22A5 | 5q23.3 |
| SLC25A13 | 7q21.3 |
| SLC25A20 | 3p21.31 |
| SLC5A5 | 19p13.11 |
| TAT | 16q22.1 |
| TAZ | Xq28 |
| TG | 8q24 |
| TPO | 2p25 |
| TSHB | 1p13 |
| TSHR | 14q31 |

TABLE 4
Exemplary Target Regions: Coding Exons

| | NCBI reference | | |
|---|---|---|---|
| Target name | sequence | Target exons | Direction |
| ABCD1 | NM_000033 | 1, 2, 3, 4, 5, 6, 7, 8, 9, 10 | 3 |
| ACAD8 | NM_014384 | 1, 2, 3, 4, 5, 6, 7, 8, 9, 10, 11 | 3 |
| ACADM | NM_000016 | 1, 3, 4, 5, 6, 7, 8, 9, 10, 11, 12 | 3 |
| ACADM | NM_001127328 | 2 | 3 |
| ACADS | NM_000017 | 1, 2, 3, 4, 5, 6, 7, 8, 9, 10 | 3 |
| ACADSB | NM_001609 | 1, 2, 3, 4, 5, 6, 7, 8, 9, 10, 11 | 3 |
| ACADVL | NM_001270447 | 1, 2 | 3 |
| ACADVL | NM_000018 | 1, 3, 4, 5, 6, 7, 8, 9, 10, 11, 12, 13, 14, 15, 16, 17, 18, 19, 20 | 3 |
| ACAT1 | NM_000019 | 1, 2, 3, 4, 5, 6, 7, 8, 9, 10, 11, 12 | 3 |
| ADA | NM_000022 | 12, 11, 10, 9, 8, 7, 6, 5, 4, 3, 2, 1 | 3 |
| AHCY | NM_000687 | 10, 9, 8, 7, 6, 5, 4, 3, 2, 1 | 3 |
| ARG1 | NM_000045 | 1, 2, 4, 5, 6, 7, 8 | 3 |
| ARG1 | NM_001244438 | 3 | 3 |
| ASL | NM_000048 | 3, 4, 5, 6, 7, 8, 9, 10, 11, 12, 13, 14, 15, 16, 17 | 3 |
| ASS1 | NM_000050 | 3, 4, 5, 6, 7, 8, 9, 10, 11, 12, 13, 14, 15, 16 | 3 |
| AUH | NM_001698 | 10, 9, 8, 7, 6, 5, 4, 3, 2, 1 | 3 |
| BCKDHA | NM_000709 | 1, 2, 3, 4, 5, 6, 7, 8, 9 | 3 |
| BCKDHB | NM_000056 | 1, 2, 3, 4, 5, 6, 7, 8, 9, 10 | 3 |
| BTD | NM_000060 | 2, 3, 4 | 3 |
| CBS | NM_000071 | 16, 15, 14, 13, 12, 11, 10, 9, 8, 7, 6, 5, 4, 3 | 3 |
| CFTR | NM_000492 | 1, 2, 3, 4, 5, 6, 7, 8, 9, 10, 11, 12, 13, 14, 15, 16, 17, 18, 19, 20, 21, 22, | 3 |
| | | 23, 24, 25, 26, 27 | |
| CPT1A | NM_001031847 | 19, 18, 17, 16, 15, 14, 13, 12, 11, 10, 9, 8, 7, 6, 5, 4, 3, 2 | 3 |
| CPT1A | NM_001876 | 19 | 3 |
| CPT2 | NM_000098 | 1, 2, 3, 4, 5 | 3 |
| CYP21A2 | NM_000500 | 1, 2, 3, 4, 5, 6, 7, 8, 9, 10 | 3 |
| DBT | NM_001918 | 11, 10, 9, 8, 7, 6, 5, 4, 3, 2, 1 | 3 |
| DECR1 | NM_001359 | 1, 2, 3, 4, 5, 6, 7, 8, 9, 10 | 3 |
| DLD | NM_000108 | 1, 2, 3, 4, 5, 6, 7, 8, 9, 10, 11, 12, 13, 14 | 3 |
| DNAJC19 | NM_145261 | 1 | 3 |
| DNAJC19 | NM_001190233 | 6, 5, 4, 3, 2, 1 | 3 |
| DUOX2 | NM_014080 | 34, 33, 32, 31, 30, 29, 28, 27, 26, 25, 24, 23, 22, 21, 20, 19, 18, 17, | 3 |
| | | 16, 15, 14, 13, 12, 11, 10, 9, 8, 7, 6, 5, 4, 3, 2, 1 | |
| ETFA | NM_000126 | 12, 11, 10, 9, 8, 7, 6, 5, 4, 3, 2, 1 | 3 |
| ETFB | NM_001985 | 2, 1 | 3 |
| ETFB | NM_001014763 | 5, 4, 3, 2 | 3 |
| ETFDH | NM_004453 | 1, 2, 4, 5, 6, 7, 8, 9, 10, 11, 12, 13 | 3 |
| FAH | NM_000137 | 1, 2, 3, 4, 5, 6, 7, 8, 9, 10, 11, 12, 13, 14 | 3 |
| GAA | NM_001079803 | 1 | 3 |
| GAA | NM_000152 | 2, 3, 4, 5, 6, 7, 8, 9, 10, 11, 12, 13, 14, 15, 16, 17, 18, 19, 20 | 3 |
| GALC | NM_001201402 | 1 | 3 |
| GALC | NM_000153 | 17, 16, 15, 14, 13, 12, 11, 10, 9, 8, 7, 6, 5, 4, 3, 2, 1 | 3 |
| GALE | NM_001008216 | 1 | 3 |
| GALE | NM_000403 | 12, 11, 10, 9, 8, 7, 6, 5, 4, 3, 2, 1 | 3 |
| GALK1 | NM_000154 | 8, 7, 6, 5, 4, 3, 2, 1 | 3 |
| GALT | NM_000155 | 1, 2, 3, 4, 5, 6, 7, 8, 9, 10, 11 | 3 |
| GBA | NM_000157 | 11, 10, 9, 8, 7, 6, 5, 4, 3, 2 | 3 |
| GBA | NM_001005742 | 2 | 3 |
| GBA | NM_001005741 | 1 | 3 |
| GCDH | NM_000159 | 1, 2, 5, 6, 7, 8, 9, 10, 11, 12 | 3 |
| GCH1 | NM_001024024 | 7 | 3 |
| GCH1 | NM_000161 | 5, 4, 3, 2, 1 | 3 |
| GJB2 | NM_004004 | 2, 1 | 3 |
| GJB3 | NM_024009 | 1, 2 | 3 |
| GJB3 | NM_001005752 | 1 | 3 |
| GJB6 | NM_001110219 | 5, 4, 3, 2, 1 | 3 |
| GJB6 | NM_006783 | 1 | 3 |
| GLA | NM_000169 | 7, 6, 5, 4, 3, 2, 1 | 3 |
| GNMT | NM_018960 | 1, 2, 3, 4, 5, 6 | 3 |
| HADH | NM_001184705 | 1, 2, 3, 4, 5, 6, 7, 8, 9 | 3 |
| HADHA | NM_000182 | 20, 19, 18, 17, 16, 15, 14, 13, 12, 11, 10, 9, 8, 7, 6, 5, 4, 3, 2, 1 | 3 |
| HADHB | NM_000183 | 1, 2, 3, 4, 5, 6, 7, 8, 9, 10, 11, 12, 13, 14, 15, 16 | 3 |
| HBA1 | NM_000558 | 1, 2, 3 | 3 |
| HBA2 | NM_000517 | 1, 2, 3 | 3 |
| HBB | NM_000518 | 3, 2, 1 | 3 |
| HLCS | NM_000411 | 12, 11, 10, 9, 8, 7, 6, 5, 4, 3, 2, 1 | 3 |
| HLCS | NM_001242784 | 3, 1 | 3 |
| HLCS | NM_001242785 | 1 | 3 |
| HMGCL | NM_000191 | 9, 8, 7, 6, 5, 4, 3, 2, 1 | 3 |
| HPD | NM_001171993 | 16, 15, 14, 13, 12, 11, 10, 9, 8, 7, 6, 5, 4, 3, 2, 1 | 3 |
| HPD | NM_002150 | 1 | 3 |
| HSD17B10 | NM_001037811 | 6, 4, 3, 2, 1 | 3 |
| HSD17B10 | NM_004493 | 5 | 3 |
| IDUA | NM_000203 | 1, 2, 3, 4, 5, 6, 7, 8, 9, 10, 11, 12, 13, 14 | 3 |
| IL2RG | NM_000206 | 8, 7, 6, 5, 4, 3, 2, 1 | 3 |
| IVD | NM_002225 | 2 | 3 |
| IVD | NM_001159508 | 1, 2, 3, 4, 5, 6, 7, 8, 9, 10, 11 | 3 |
| MAT1A | NM_000429 | 9, 8, 7, 6, 5, 4, 3, 2, 1 | 3 |
| MCCC1 | NM_020166 | 19, 18, 17, 16, 15, 14, 13, 12, 11, 10, 9, 8, 7, 6, 5, 4, 3, 2, 1 | 3 |
| MCCC2 | NM_022132 | 1, 2, 3, 4, 5, 6, 7, 8, 9, 10, 11, 12, 13, 14, 15, 16, 17 | 3 |
| MCEE | NM_032601 | 3, 2, 1 | 3 |
| MLYCD | NM_012213 | 1, 2, 3, 4, 5 | 3 |
| MMAA | NM_172250 | 1, 2, 3, 4, 5, 6, 7 | 3 |
| MMAB | NM_052845 | 9, 8, 7, 6, 5, 4, 3, 2, 1 | 3 |
| MMADHC | NM_015702 | 8, 7, 6, 5, 4, 3, 2, 1 | 3 |
| MTHFR | NM_005957 | 12, 11, 10, 9, 8, 7, 6, 5, 4, 3, 2, 1 | 3 |
| MTR | NM_000254 | 1, 2, 3, 4, 5, 6, 7, 8, 9, 10, 11, 12, 13, 14, 15, 16, 17, 18, 19, 20, 21, 22, 23, | 3 |
| | | 24, 25, 26, 27, 28, 29, 30, 31, 32, 33 | |
| MTRR | NM_002454 | 2, 3, 4, 5, 6, 7, 8, 9, 10, 11, 12, 13, 14, 15 | 3 |
| MTRR | NM_024010 | 1 | 3 |
| MUT | NM_000255 | 13, 12, 11, 10, 9, 8, 7, 6, 5, 4, 3, 2, 1 | 3 |
| NGLY1 | NM_001145294 | 7, 1 | 3 |
| NGLY1 | NM_001145293 | 12, 11, 10, 9, 8, 7, 6, 5, 4, 3, 2, 1 | 3 |
| NPC1 | NM_000271 | 25, 24, 23, 22, 21, 20, 19, 18, 17, 16, 15, 14, 13, 12, 11, 10, 9, 8, 7, 6, | 3 |
| | | 5, 4, 3, 2, 1 | |
| NPC2 | NM_006432 | 5, 4, 3, 2, 1 | 3 |
| OPA3 | NM_001017989 | 2, 1 | 3 |
| OPA3 | NM_025136 | 2 | 3 |
| PAH | NM_000277 | 13, 12, 11, 10, 9, 8, 7, 6, 5, 4, 3, 2, 1 | 3 |
| PAX8 | NM_003466 | 12, 11, 10, 8, 7, 6, 5, 4, 3, 2, 1 | 3 |
| PAX8 | NM_013952 | 9 | 3 |
| PCBD1 | NM_000281 | 4, 3, 2, 1 | 3 |
| PCCA | NM_000282 | 1, 2, 3, 4, 5, 6, 7, 8, 9, 10, 11, 12, 13, 14, 15, 16, 17, 18, 19, 20, 21, 22, | 3 |
| | | 23, 24 | |
| PCCB | NM_001178014 | 4 | 3 |
| PCCB | NM_000532 | 1, 2, 3, 4, 5, 6, 7, 8, 9, 10, 11, 12, 13, 14, 15 | 3 |
| PTS | NM_000317 | 1, 2, 3, 4, 5, 6 | 3 |
| QDPR | NM_000320 | 7, 6, 5, 4, 3, 2, 1 | 3 |
| SLC22A5 | NM_003060 | 1, 2, 3, 4, 5, 6, 7, 8, 9, 10 | 3 |
| SLC25A13 | NM_014251 | 10 | 3 |
| SLC25A13 | NM_001160210 | 18, 17, 16, 15, 14, 13, 12, 11, 9, 8, 7, 6, 5, 4, 3, 2, 1 | 3 |
| SLC25A20 | NM_000387 | 9, 8, 7, 6, 5, 4, 3, 2, 1 | 3 |
| SLC5A5 | NM_000453 | 1, 2, 3, 4, 5, 6, 7, 8, 9, 10, 11, 12, 13, 14, 15 | 3 |
| TAT | NM_000353 | 12, 11, 10, 9, 8, 7, 6, 5, 4, 3, 2, 1 | 3 |
| TAZ | NM_000116 | 1, 2, 3, 4, 5, 6, 7, 8, 9, 10, 11 | 3 |
| TG | NM_003235 | 1, 2, 3, 4, 5, 6, 7, 8, 9, 10, 11, 12, 13, 14, 15, 16, 17, 18, 19, 20, 21, 22, 23, | 3 |
| | | 24, 25, 26, 27, 28, 29, 30, 31, 32, 33, 34, 35, 36, 37, 38, 39, 40, 41, | |
| | | 42, 43, 44, 45, 46, 47, 48 | |
| TPO | NM_001206744 | 1 | 3 |
| TPO | NM_000547 | 2, 3, 4, 5, 6, 7, 8, 9, 10, 11, 12, 13, 14, 15, 16, 17 | 3 |
| TSHB | NM_001277991 | 1 | 3 |
| TSHB | NM_000549 | 1, 2 | 3 |
| TSHR | NM_001142626 | 9 | 3 |
| TSHR | NM_000369 | 1, 2, 3, 4, 5, 6, 7, 8, 9, 10 | 3 |

TABLE 5
Exemplary Gene Variant Locations for Screening

| | Chrom. | Location | Location | | |
|---|---|---|---|---|---|
| Gene | #: | Start | End | ID | Type |
| ABCD1 | X | 152990721 | 152990729 | RCV000012069 | deletion |
| ABCD1 | X | 152990752 | 152990767 | RCV000077962 | deletion |
| ABCD1 | X | 152991067 | 152991067 | RCV000077963 | SNV |
| ABCD1 | X | 152991127 | 152991127 | RCV000077964 | SNV |
| ABCD1 | X | 152991142 | 152991142 | RCV000029289; RCV000077965 | SNV |
| ABCD1 | X | 152991164 | 152991164 | RCV000012049 | SNV |
| ABCD1 | X | 152991219 | 152991241 | RCV000077966 | deletion |
| ABCD1 | X | 152991241 | 152991241 | RCV000012050 | SNV |
| ABCD1 | X | 152991335 | 152991335 | RCV000077967 | SNV |
| ABCD1 | X | 152991517 | 152991517 | RCV000012051 | SNV |
| ABCD1 | X | 152991559 | 152991559 | RCV000029290 | SNV |
| ABCD1 | X | 152991592 | 152991592 | RCV000012044 | SNV |
| ABCD1 | X | 152991592 | 152991594 | RCV000012066 | deletion |
| ABCD1 | X | 153001649 | 153001649 | RCV000012048 | SNV |
| ABCD1 | X | 153001686 | 153001686 | RCV000012052 | SNV |
| ABCD1 | X | 153001826 | 153001826 | RCV000012053 | SNV |
| ABCD1 | X | 153001940 | 153001941 | RCV000029284 | duplication |
| ABCD1 | X | 153001964 | 153001964 | RCV000012054 | SNV |
| ABCD1 | X | 153002613 | 153002613 | RCV000077950 | SNV |
| ABCD1 | X | 153002632 | 153002633 | RCV000012055; RCV000077951 | deletion |
| ABCD1 | X | 153002646 | 153002646 | RCV000012056 | SNV |
| ABCD1 | X | 153002668 | 153002668 | RCV000012045 | SNV |
| ABCD1 | X | 153005601 | 153005601 | RCV000012057 | SNV |
| ABCD1 | X | 153005609 | 153005609 | RCV000012058 | deletion |
| ABCD1 | X | 153005609 | 153005609 | RCV000012059; RCV000077954 | SNV |
| ABCD1 | X | 153005610 | 153005610 | RCV000077955 | SNV |
| ABCD1 | X | 153005643 | 153005643 | RCV000077956 | SNV |
| ABCD1 | X | 153005649 | 153005649 | RCV000029285 | SNV |
| ABCD1 | X | 153006053 | 153006053 | RCV000077957 | SNV |
| ABCD1 | X | 153006072 | 153006072 | RCV000077958 | SNV |
| ABCD1 | X | 153006164 | 153006164 | RCV000077959 | SNV |
| ABCD1 | X | 153008462 | 153008462 | RCV000077960 | SNV |
| ABCD1 | X | 153008477 | 153008477 | RCV000012062 | SNV |
| ABCD1 | X | 153008509 | 153008509 | RCV000012065 | SNV |
| ABCD1 | X | 153008510 | 153008510 | RCV000012064 | SNV |
| ACAD8 | 11 | 134128412 | 134128412 | RCV000081617 | SNV |
| ACAD8 | 11 | 134128475 | 134128476 | RCV000081619 | duplication |
| ACAD8 | 11 | 134128483 | 134128483 | RCV000005686 | SNV |
| ACAD8 | 11 | 134131194 | 134131194 | RCV000005689 | SNV |
| ACAD8 | 11 | 134131232 | 134131232 | RCV000005690 | SNV |
| ACAD8 | 11 | 134131680 | 134131680 | RCV000005688 | SNV |
| ACAD8 | 11 | 134132450 | 134132450 | RCV000005687; RCV000081615 | SNV |
| ACADM | 1 | 76198337 | 76198337 | RCV000077881 | SNV |
| ACADM | 1 | 76198367 | 76198367 | RCV000077882 | SNV |
| ACADM | 1 | 76198409 | 76198409 | RCV000003780 | SNV |
| ACADM | 1 | 76198554 | 76198554 | RCV000077885 | SNV |
| ACADM | 1 | 76199288 | 76199288 | RCV000003782 | SNV |
| ACADM | 1 | 76200535 | 76200535 | RCV000003774 | SNV |
| ACADM | 1 | 76205773 | 76205773 | RCV000003778 | SNV |
| ACADM | 1 | 76205779 | 76205779 | RCV000003777; RCV000077891 | SNV |
| ACADM | 1 | 76211507 | 76211507 | RCV000077892 | SNV |
| ACADM | 1 | 76211508 | 76211508 | RCV000077893 | SNV |
| ACADM | 1 | 76215125 | 76215125 | RCV000003773 | SNV |
| ACADM | 1 | 76215129 | 76215129 | RCV000003781; RCV000077894 | SNV |
| ACADM | 1 | 76215194 | 76215194 | RCV000003771 | SNV |
| ACADM | 1 | 76215237 | 76215237 | RCV000003779 | SNV |
| ACADM | 1 | 76226846 | 76226846 | RCV000003769; RCV000077895 | SNV |
| ACADM | 1 | 76226963 | 76226966 | RCV000003775; RCV000077877 | deletion |
| ACADM | 1 | 76226985 | 76226985 | RCV000003772 | SNV |
| ACADS | 12 | 121164918 | 121164918 | RCV000004029 | SNV |
| ACADS | 12 | 121164946 | 121164946 | RCV000023585 | SNV |
| ACADS | 12 | 121174846 | 121174846 | RCV000004036 | SNV |
| ACADS | 12 | 121174852 | 121174852 | RCV000004031 | SNV |
| ACADS | 12 | 121174888 | 121174890 | RCV000004037 | deletion |
| ACADS | 12 | 121174897 | 121174897 | RCV000004030 | SNV |
| ACADS | 12 | 121174901 | 121174901 | RCV000023587 | SNV |
| ACADS | 12 | 121175678 | 121175678 | RCV000004034 | SNV |
| ACADS | 12 | 121175696 | 121175696 | RCV000004032 | SNV |
| ACADS | 12 | 121175742 | 121175742 | RCV000004038 | SNV |
| ACADS | 12 | 121176083 | 121176083 | RCV000004035; RCV000077896 | SNV |
| ACADS | 12 | 121176662 | 121176662 | RCV000004039 | SNV |
| ACADS | 12 | 121176944 | 121176944 | RCV000023586 | SNV |
| ACADS | 12 | 121176971 | 121176971 | RCV000004040 | SNV |
| ACADS | 12 | 121177150 | 121177150 | RCV000004041 | SNV |
| ACADS | 12 | 121177159 | 121177159 | RCV000004033 | SNV |
| ACADSB | 10 | 124800121 | 124800121 | RCV000009780 | SNV |
| ACADSB | 10 | 124802643 | 124802643 | RCV000009778 | SNV |
| ACADSB | 10 | 124812607 | 124812607 | RCV000009781 | SNV |
| ACADSB | 10 | 124812676 | 124812676 | RCV000009777 | SNV |
| ACADVL | 17 | 7123838 | 7123838 | RCV000001698; RCV000020076; | SNV |
| | | | | RCV000077913 | |
| ACADVL | 17 | 7123838 | 7123838 | RCV000001698; RCV000020076; | SNV |
| | | | | RCV000077913 | |
| ACADVL | 17 | 7124242 | 7124242 | RCV000001692 | deletion |
| ACADVL | 17 | 7124243 | 7124243 | RCV000001691; RCV000077915 | deletion |
| ACADVL | 17 | 7124288 | 7124290 | RCV000001693 | deletion |
| ACADVL | 17 | 7124899 | 7124899 | RCV000077919 | SNV |
| ACADVL | 17 | 7125387 | 7125387 | RCV000001698 | SNV |
| ACADVL | 17 | 7125522 | 7125522 | RCV000020080 | SNV |
| ACADVL | 17 | 7125591 | 7125591 | RCV000020081; RCV000077925 | SNV |
| ACADVL | 17 | 7126002 | 7126004 | RCV000001694 | deletion |
| ACADVL | 17 | 7126003 | 7126005 | RCV000077926 | deletion |
| ACADVL | 17 | 7126480 | 7126480 | RCV000077900 | SNV |
| ACADVL | 17 | 7126518 | 7126518 | RCV000001695 | SNV |
| ACADVL | 17 | 7127006 | 7127006 | RCV000020069 | SNV |
| ACADVL | 17 | 7127026 | 7127026 | RCV000001700 | SNV |
| ACADVL | 17 | 7127184 | 7127184 | RCV000020072; RCV000077903 | SNV |
| ACADVL | 17 | 7127303 | 7127303 | RCV000001701 | SNV |
| ACADVL | 17 | 7127326 | 7127326 | RCV000001699 | SNV |
| ACADVL | 17 | 7127343 | 7127343 | RCV000077905 | duplication |
| ACADVL | 17 | 7127359 | 7127359 | RCV000020073 | SNV |
| ACADVL | 17 | 7127360 | 7127360 | RCV000077906 | SNV |
| ACADVL | 17 | 7128285 | 7128285 | RCV000001690 | SNV |
| ACAT1 | 11 | 107992335 | 107992335 | RCV000002972 | SNV |
| ACAT1 | 11 | 108004987 | 108004987 | RCV000002977 | SNV |
| ACAT1 | 11 | 108005967 | 108005967 | RCV000002980 | SNV |
| ACAT1 | 11 | 108009661 | 108009661 | RCV000077931 | SNV |
| ACAT1 | 11 | 108009736 | 108009736 | RCV000002967 | SNV |
| ACAT1 | 11 | 108012415 | 108012415 | RCV000002974; RCV000077933 | SNV |
| ACAT1 | 11 | 108013242 | 108013242 | RCV000077934 | deletion |
| ACAT1 | 11 | 108013272 | 108013272 | RCV000002978 | SNV |
| ACAT1 | 11 | 108014766 | 108014766 | RCV000002979 | SNV |
| ACAT1 | 11 | 108016956 | 108016958 | RCV000002975 | deletion |
| ACAT1 | 11 | 108017006 | 108017007 | RCV000002976 | duplication |
| ACAT1 | 11 | 108017059 | 108017059 | RCV000002973 | SNV |
| ACAT1 | 11 | 108017061 | 108017061 | RCV000002966 | SNV |
| ADA | 20 | 43249032 | 43249032 | RCV000002036 | SNV |
| ADA | 20 | 43249723 | 43249723 | RCV000002035 | SNV |
| ADA | 20 | 43249744 | 43249744 | RCV000059115; RCV000002038 | SNV |
| ADA | 20 | 43249762 | 43249762 | RCV000002048 | SNV |
| ADA | 20 | 43249762 | 43249762 | RCV000029302 | SNV |
| ADA | 20 | 43251253 | 43251253 | RCV000059114; RCV000002041 | SNV |
| ADA | 20 | 43251552 | 43251552 | RCV000059113; RCV000002057 | SNV |
| ADA | 20 | 43251680 | 43251680 | RCV000002045 | SNV |
| ADA | 20 | 43251683 | 43251683 | RCV000059112; RCV000002044 | SNV |
| ADA | 20 | 43251694 | 43251694 | RCV000002034 | SNV |
| ADA | 20 | 43251695 | 43251695 | RCV000059111; RCV000002043 | SNV |
| ADA | 20 | 43254221 | 43254221 | RCV000059106; RCV000002061 | SNV |
| ADA | 20 | 43254222 | 43254222 | RCV000002047 | SNV |
| ADA | 20 | 43254234 | 43254234 | RCV000059105; RCV000002056 | SNV |
| ADA | 20 | 43254242 | 43254242 | RCV000059104; RCV000002040 | SNV |
| ADA | 20 | 43255139 | 43255139 | RCV000002042 | SNV |
| ADA | 20 | 43255143 | 43255143 | RCV000002058 | SNV |
| ADA | 20 | 43255157 | 43255157 | RCV000002033 | SNV |
| ADA | 20 | 43255158 | 43255158 | RCV000002032 | SNV |
| ADA | 20 | 43255169 | 43255169 | RCV000002058 | SNV |
| ADA | 20 | 43255233 | 43255233 | RCV000059096; RCV000002039 | SNV |
| ADA | 20 | 43255238 | 43255238 | RCV000002054 | SNV |
| ADA | 20 | 43280227 | 43280227 | RCV000002050 | SNV |
| AHCY | 20 | 32880181 | 32880181 | RCV000013819 | SNV |
| AHCY | 20 | 32880273 | 32880273 | RCV000013818 | SNV |
| ARG1 | 6 | 131894454 | 131894454 | RCV000002494 | SNV |
| ASL | 7 | 65546812 | 65546812 | RCV000078010 | SNV |
| ASL | 7 | 65547430 | 65547430 | RCV000002499 | SNV |
| ASL | 7 | 65547921 | 65547921 | RCV000020416 | SNV |
| ASL | 7 | 65551738 | 65551738 | RCV000002503 | SNV |
| ASL | 7 | 65551750 | 65551750 | RCV000078012 | SNV |
| ASL | 7 | 65554101 | 65554101 | RCV000002500; RCV000078017 | SNV |
| ASL | 7 | 65554680 | 65554680 | RCV000020415; RCV000078007 | SNV |
| ASL | 7 | 65557065 | 65557065 | RCV000002504 | SNV |
| ASL | 7 | 65557553 | 65557553 | RCV000002502 | SNV |
| ASS1 | 9 | 133327655 | 133327655 | RCV000006696 | SNV |
| ASS1 | 9 | 133327668 | 133327668 | RCV000006703 | SNV |
| ASS1 | 9 | 133333869 | 133333869 | RCV000006704 | SNV |
| ASS1 | 9 | 133333936 | 133333936 | RCV000006706 | SNV |
| ASS1 | 9 | 133342161 | 133342161 | RCV000006697 | SNV |
| ASS1 | 9 | 133346260 | 133346260 | RCV000006707 | SNV |
| ASS1 | 9 | 133346264 | 133346264 | RCV000006698 | SNV |
| ASS1 | 9 | 133355785 | 133355785 | RCV000078024 | SNV |
| ASS1 | 9 | 133355791 | 133355791 | RCV000078025 | SNV |
| ASS1 | 9 | 133355792 | 133355792 | RCV000078026 | SNV |
| ASS1 | 9 | 133355833 | 133355833 | RCV000006705 | SNV |
| ASS1 | 9 | 133355834 | 133355834 | RCV000078027 | SNV |
| ASS1 | 9 | 133364791 | 133364791 | RCV000006702 | SNV |
| ASS1 | 9 | 133364809 | 133364809 | RCV000006711 | SNV |
| ASS1 | 9 | 133364851 | 133364851 | RCV000006699 | SNV |
| ASS1 | 9 | 133370368 | 133370368 | RCV000006708 | SNV |
| ASS1 | 9 | 133370370 | 133370370 | RCV000006700 | SNV |
| ASS1 | 9 | 133374932 | 133374932 | RCV000006701; RCV000078019 | SNV |
| AUH | 9 | 93976659 | 93976659 | RCV000022984 | SNV |
| AUH | 9 | 94058308 | 94058308 | RCV000022983 | SNV |
| AUH | 9 | 94060275 | 94060275 | RCV000009623 | SNV |
| AUH | 9 | 94060305 | 94060305 | RCV000022982 | SNV |
| BCKDHA | 19 | 41903746 | 41903746 | RCV000079231 | deletion |
| BCKDHA | 19 | 41916550 | 41916550 | RCV000079223 | deletion |
| BCKDHA | 19 | 41916909 | 41916909 | RCV000079238 | SNV |
| BCKDHA | 19 | 41925187 | 41925187 | RCV000079247 | SNV |
| BCKDHA | 19 | 41928081 | 41928081 | RCV000079249 | SNV |
| BCKDHA | 19 | 41928162 | 41928163 | RCV000079252 | duplication |
| BCKDHA | 19 | 41928167 | 41928167 | RCV000002479 | SNV |
| BCKDHA | 19 | 41928183 | 41928183 | RCV000079253 | SNV |
| BCKDHA | 19 | 41928214 | 41928214 | RCV000002481 | SNV |
| BCKDHA | 19 | 41928215 | 41928215 | RCV000002478 | SNV |
| BCKDHA | 19 | 41928275 | 41928275 | RCV000079257 | SNV |
| BCKDHA | 19 | 41928548 | 41928548 | RCV000002475; RCV000079258 | SNV |
| BCKDHA | 19 | 41928585 | 41928585 | RCV000079260 | SNV |
| BCKDHA | 19 | 41928589 | 41928590 | RCV000079261 | deletion |
| BCKDHA | 19 | 41928597 | 41928597 | RCV000079263 | deletion |
| BCKDHA | 19 | 41928609 | 41928609 | RCV000002480; RCV000079264 | SNV |
| BCKDHA | 19 | 41928644 | 41928644 | RCV000079265 | SNV |
| BCKDHA | 19 | 41928659 | 41928659 | RCV000079269 | SNV |
| BCKDHA | 19 | 41928943 | 41928943 | RCV000079216 | SNV |
| BCKDHA | 19 | 41930401 | 41930401 | RCV000002476; RCV000002477 | SNV |
| BCKDHA | 19 | 41930409 | 41930409 | RCV000079225 | SNV |
| BCKDHA | 19 | 41930477 | 41930477 | RCV000079227 | SNV |
| BCKDHA | 19 | 41930485 | 41930486 | RCV000079228 | deletion |
| BCKDHA | 19 | 41930487 | 41930487 | RCV000055825; RCV000002473; | SNV |
| | | | | RCV000079229 | |
| BCKDHA | 19 | 41930489 | 41930489 | RCV000079230 | SNV |
| BCKDHB | 6 | 80816443 | 80816444 | RCV000082736 | deletion |
| BCKDHB | 6 | 80816503 | 80816513 | RCV000082778 | deletion |
| BCKDHB | 6 | 80838905 | 80838905 | RCV000082734 | SNV |
| BCKDHB | 6 | 80838945 | 80838945 | RCV000082737 | SNV |
| BCKDHB | 6 | 80877394 | 80877394 | RCV000082738 | SNV |
| BCKDHB | 6 | 80877407 | 80877407 | RCV000012717 | SNV |
| BCKDHB | 6 | 80878593 | 80878593 | RCV000082741 | SNV |
| BCKDHB | 6 | 80878602 | 80878602 | RCV000082742 | SNV |
| BCKDHB | 6 | 80878622 | 80878622 | RCV000082746 | SNV |
| BCKDHB | 6 | 80878622 | 80878622 | RCV000082747 | SNV |
| BCKDHB | 6 | 80878622 | 80878622 | RCV000082748 | SNV |
| BCKDHB | 6 | 80878623 | 80878623 | RCV000082749 | SNV |
| BCKDHB | 6 | 80878640 | 80878640 | RCV000082751 | SNV |
| BCKDHB | 6 | 80878661 | 80878661 | RCV000082753 | SNV |
| BCKDHB | 6 | 80878662 | 80878662 | RCV000012715; RCV000056008; | SNV |
| | | | | RCV000082754 | |
| BCKDHB | 6 | 80878706 | 80878707 | RCV000082757 | deletion |
| BCKDHB | 6 | 80878709 | 80878710 | RCV000082758 | deletion |
| BCKDHB | 6 | 80878730 | 80878730 | RCV000012716 | SNV |
| BCKDHB | 6 | 80910656 | 80910656 | RCV000082766 | SNV |
| BCKDHB | 6 | 80910660 | 80910660 | RCV000082767 | SNV |
| BCKDHB | 6 | 80910707 | 80910707 | RCV000082768 | SNV |
| BCKDHB | 6 | 80910740 | 80910740 | RCV000056009; RCV000082770 | SNV |
| BCKDHB | 6 | 80912831 | 80912831 | RCV000082775 | SNV |
| BCKDHB | 6 | 80912862 | 80912862 | RCV000082776 | deletion |
| BCKDHB | 6 | 80912880 | 80912880 | RCV000082777 | SNV |
| BCKDHB | 6 | 80982851 | 80982851 | RCV000082780 | SNV |
| BCKDHB | 6 | 80982870 | 80982870 | RCV000082781 | SNV |
| BCKDHB | 6 | 80982916 | 80982916 | RCV000082720 | SNV |
| BCKDHB | 6 | 81053388 | 81053388 | RCV000082721 | SNV |
| BCKDHB | 6 | 81053456 | 81053456 | RCV000056007; RCV000082726 | SNV |
| BTD | 3 | 15676984 | 15676990 | RCV000001972; RCV000078084; | indel |
| | | | | RCV000021886 | |
| BTD | 3 | 15676984 | 15676990 | RCV000001972; RCV000078084; | indel |
| | | | | RCV000021886 | |
| BTD | 3 | 15676986 | 15676986 | RCV000001976 | SNV |
| BTD | 3 | 15677014 | 15677014 | RCV000022030 | SNV |
| BTD | 3 | 15677019 | 15677019 | RCV000021888; RCV000021890; | SNV |
| | | | | RCV000021889 | |
| BTD | 3 | 15677019 | 15677019 | RCV000021888; RCV000021890; | SNV |
| | | | | RCV000021889 | |
| BTD | 3 | 15677019 | 15677019 | RCV000021888; RCV000021890; | SNV |
| | | | | RCV000021889 | |
| BTD | 3 | 15677022 | 15677022 | RCV000021891 | SNV |
| BTD | 3 | 15677045 | 15677045 | RCV000021892 | SNV |
| BTD | 3 | 15677046 | 15677046 | RCV000021892 | SNV |
| BTD | 3 | 15677057 | 15677057 | RCV000021893 | SNV |
| BTD | 3 | 15677070 | 15677070 | RCV000021894 | SNV |
| BTD | 3 | 15677070 | 15677070 | RCV000021895 | SNV |
| BTD | 3 | 15677076 | 15677076 | RCV000021896 | SNV |
| BTD | 3 | 15677078 | 15677078 | RCV000032017 | SNV |
| BTD | 3 | 15677080 | 15677080 | RCV000021898 | SNV |
| BTD | 3 | 15677098 | 15677098 | RCV000021900; RCV000021886; | SNV |
| | | | | RCV000021901 | |
| BTD | 3 | 15677098 | 15677098 | RCV000021900; RCV000021886; | SNV |
| | | | | RCV000021901 | |
| BTD | 3 | 15677098 | 15677098 | RCV000021900; RCV000021886; | SNV |
| | | | | RCV000021901 | |
| BTD | 3 | 15677121 | 15677121 | RCV000001982; RCV000021903; | SNV |
| | | | | RCV000032009 | |
| BTD | 3 | 15677121 | 15677121 | RCV000001982; RCV000021903; | SNV |
| | | | | RCV000032009 | |
| BTD | 3 | 15677121 | 15677121 | RCV000001982; RCV000021903; | SNV |
| | | | | RCV000032009 | |
| BTD | 3 | 15677122 | 15677122 | RCV000021901; RCV000021905 | SNV |
| BTD | 3 | 15677122 | 15677122 | RCV000021901; RCV000021905 | SNV |
| BTD | 3 | 15677131 | 15677131 | RCV000021906 | SNV |
| BTD | 3 | 15677131 | 15677131 | RCV000021907 | SNV |
| BTD | 3 | 15677132 | 15677140 | RCV000021908 | deletion |
| BTD | 3 | 15677134 | 15677134 | RCV000021909 | SNV |
| BTD | 3 | 15677148 | 15677148 | RCV000021910 | SNV |
| BTD | 3 | 15677164 | 15677164 | RCV000021911 | SNV |
| BTD | 3 | 15677169 | 15677169 | RCV000021912 | SNV |
| BTD | 3 | 15677184 | 15677184 | RCV000021913 | SNV |
| BTD | 3 | 15683415 | 15683415 | RCV000021914 | SNV |
| BTD | 3 | 15683431 | 15683431 | RCV000021915 | SNV |
| BTD | 3 | 15683439 | 15683439 | RCV000021916 | SNV |
| BTD | 3 | 15683439 | 15683439 | RCV000021917 | SNV |
| BTD | 3 | 15683446 | 15683446 | RCV000021918 | SNV |
| BTD | 3 | 15683461 | 15683461 | RCV000021919 | SNV |
| BTD | 3 | 15683469 | 15683469 | RCV000021920 | SNV |
| BTD | 3 | 15683487 | 15683487 | RCV000021921 | SNV |
| BTD | 3 | 15683498 | 15683498 | RCV000021922 | deletion |
| BTD | 3 | 15683512 | 15683513 | RCV000032021 | duplication |
| BTD | 3 | 15683529 | 15683529 | RCV000021923 | SNV |
| BTD | 3 | 15683548 | 15683548 | RCV000021924 | SNV |
| BTD | 3 | 15683550 | 15683550 | RCV000021926 | SNV |
| BTD | 3 | 15683559 | 15683559 | RCV000021927 | SNV |
| BTD | 3 | 15683564 | 15683564 | RCV000021928 | SNV |
| BTD | 3 | 15685829 | 15685829 | RCV000021930 | SNV |
| BTD | 3 | 15685832 | 15685832 | RCV000021931 | SNV |
| BTD | 3 | 15685833 | 15685833 | RCV000021904; RCV000078072; | SNV |
| | | | | RCV000032009; RCV000021933 | |
| BTD | 3 | 15685833 | 15685833 | RCV000021904; RCV000078072; | SNV |
| | | | | RCV000032009; RCV000021933 | |
| BTD | 3 | 15685833 | 15685833 | RCV000021904; RCV000078072; | SNV |
| | | | | RCV000032009; RCV000021933 | |
| BTD | 3 | 15685848 | 15685848 | RCV000021934 | SNV |
| BTD | 3 | 15685853 | 15685854 | RCV000021935 | deletion |
| BTD | 3 | 15685874 | 15685874 | RCV000021936; RCV000031859; | SNV |
| | | | | RCV000078073 | |
| BTD | 3 | 15685874 | 15685874 | RCV000021936; RCV000031859; | SNV |
| | | | | RCV000078073 | |
| BTD | 3 | 15685878 | 15685878 | RCV000021937 | SNV |
| BTD | 3 | 15685881 | 15685881 | RCV000032018 | SNV |
| BTD | 3 | 15685891 | 15685891 | RCV000021938; RCV000078074 | SNV |
| BTD | 3 | 15685907 | 15685907 | RCV000021939 | deletion |
| BTD | 3 | 15685920 | 15685920 | RCV000021940; RCV000078075 | SNV |
| BTD | 3 | 15685922 | 15685922 | RCV000021941 | SNV |
| BTD | 3 | 15685946 | 15685946 | RCV000021942 | SNV |
| BTD | 3 | 15685947 | 15685947 | RCV000021943 | SNV |
| BTD | 3 | 15685950 | 15685950 | RCV000021944 | SNV |
| BTD | 3 | 15685957 | 15685957 | RCV000021946 | deletion |
| BTD | 3 | 15685957 | 15685959 | RCV000021945 | deletion |
| BTD | 3 | 15685958 | 15685958 | RCV000021947 | SNV |
| BTD | 3 | 15685968 | 15685968 | RCV000021948 | SNV |
| BTD | 3 | 15685992 | 15685992 | RCV000021949 | SNV |
| BTD | 3 | 15685994 | 15685994 | RCV000021950 | SNV |
| BTD | 3 | 15685995 | 15685995 | RCV000021951; RCV000078077 | SNV |
| BTD | 3 | 15686004 | 15686004 | RCV000021952; RCV000032022 | SNV |
| BTD | 3 | 15686004 | 15686004 | RCV000021952; RCV000032022 | SNV |
| BTD | 3 | 15686006 | 15686006 | RCV000021953 | SNV |
| BTD | 3 | 15686015 | 15686015 | RCV000021955 | SNV |
| BTD | 3 | 15686017 | 15686017 | RCV000021956 | SNV |
| BTD | 3 | 15686027 | 15686027 | RCV000022031 | SNV |
| BTD | 3 | 15686045 | 15686045 | RCV000021957 | SNV |
| BTD | 3 | 15686072 | 15686072 | RCV000021958 | SNV |
| BTD | 3 | 15686097 | 15686097 | RCV000021959 | SNV |
| BTD | 3 | 15686106 | 15686106 | RCV000021960 | SNV |
| BTD | 3 | 15686118 | 15686118 | RCV000001978 | SNV |
| BTD | 3 | 15686120 | 15686120 | RCV000021961 | SNV |
| BTD | 3 | 15686121 | 15686121 | RCV000032019 | SNV |
| BTD | 3 | 15686127 | 15686127 | RCV000021962 | SNV |
| BTD | 3 | 15686157 | 15686157 | RCV000021963; RCV000021964 | SNV |
| BTD | 3 | 15686157 | 15686157 | RCV000021963; RCV000021964 | SNV |
| BTD | 3 | 15686177 | 15686177 | RCV000021965 | SNV |
| BTD | 3 | 15686195 | 15686195 | RCV000021966 | SNV |
| BTD | 3 | 15686196 | 15686196 | RCV000021967 | SNV |
| BTD | 3 | 15686199 | 15686199 | RCV000021968 | SNV |
| BTD | 3 | 15686228 | 15686228 | RCV000021889 | SNV |
| BTD | 3 | 15686243 | 15686243 | RCV000021969 | SNV |
| BTD | 3 | 15686250 | 15686250 | RCV000021970 | SNV |
| BTD | 3 | 15686259 | 15686259 | RCV000021971 | SNV |
| BTD | 3 | 15686292 | 15686292 | RCV000021972 | SNV |
| BTD | 3 | 15686295 | 15686295 | RCV000021973 | SNV |
| BTD | 3 | 15686296 | 15686296 | RCV000021964; RCV000021975 | SNV |
| BTD | 3 | 15686296 | 15686296 | RCV000021964; RCV000021975 | SNV |
| BTD | 3 | 15686296 | 15686296 | RCV000021974; RCV000078082 | deletion |
| BTD | 3 | 15686297 | 15686297 | RCV000021976 | SNV |
| BTD | 3 | 15686298 | 15686298 | RCV000021977 | SNV |
| BTD | 3 | 15686331 | 15686331 | RCV000021978; RCV000078083 | SNV |
| BTD | 3 | 15686364 | 15686364 | RCV000022032 | SNV |
| BTD | 3 | 15686409 | 15686409 | RCV000022033 | SNV |
| BTD | 3 | 15686412 | 15686412 | RCV000021979 | deletion |
| BTD | 3 | 15686415 | 15686415 | RCV000021980 | deletion |
| BTD | 3 | 15686459 | 15686459 | RCV000021981 | SNV |
| BTD | 3 | 15686469 | 15686469 | RCV000021982 | SNV |
| BTD | 3 | 15686520 | 15686520 | RCV000021983 | SNV |
| BTD | 3 | 15686521 | 15686521 | RCV000032015 | SNV |
| BTD | 3 | 15686534 | 15686534 | RCV000021984; RCV000021985 | SNV |
| BTD | 3 | 15686534 | 15686534 | RCV000021984; RCV000021985 | SNV |
| BTD | 3 | 15686554 | 15686555 | RCV000021986 | deletion |
| BTD | 3 | 15686568 | 15686568 | RCV000021987 | SNV |
| BTD | 3 | 15686570 | 15686570 | RCV000001981 | SNV |
| BTD | 3 | 15686574 | 15686574 | RCV000021990 | SNV |
| BTD | 3 | 15686577 | 15686577 | RCV000021991 | SNV |
| BTD | 3 | 15686590 | 15686604 | RCV000021992 | indel |
| BTD | 3 | 15686600 | 15686600 | RCV000032020 | SNV |
| BTD | 3 | 15686602 | 15686602 | RCV000021993 | deletion |
| BTD | 3 | 15686602 | 15686613 | RCV000021988 | deletion |
| BTD | 3 | 15686603 | 15686614 | RCV000032016 | deletion |
| BTD | 3 | 15686612 | 15686612 | RCV000021995 | SNV |
| BTD | 3 | 15686615 | 15686615 | RCV000021994 | SNV |
| BTD | 3 | 15686616 | 15686616 | RCV000021996 | SNV |
| BTD | 3 | 15686627 | 15686627 | RCV000021997 | duplication |
| BTD | 3 | 15686630 | 15686630 | RCV000021998 | SNV |
| BTD | 3 | 15686631 | 15686631 | RCV000021999 | SNV |
| BTD | 3 | 15686634 | 15686634 | RCV000021890; RCV000022001 | SNV |
| BTD | 3 | 15686634 | 15686634 | RCV000021890; RCV000022001 | SNV |
| BTD | 3 | 15686634 | 15686634 | RCV000022000 | SNV |
| BTD | 3 | 15686638 | 15686638 | RCV000022002 | SNV |
| BTD | 3 | 15686647 | 15686647 | RCV000022003 | SNV |
| BTD | 3 | 15686676 | 15686676 | RCV000022004 | SNV |
| BTD | 3 | 15686677 | 15686677 | RCV000022005 | SNV |
| BTD | 3 | 15686693 | 15686693 | RCV000001977; RCV000078064; | SNV |
| | | | | RCV000001981; RCV000021912; | |
| | | | | RCV000021936; RCV000021952; | |
| | | | | RCV000021933 | |
| BTD | 3 | 15686693 | 15686693 | RCV000001977; RCV000078064; | SNV |
| | | | | RCV000001981; RCV000021912; | |
| | | | | RCV000021936; RCV000021952; | |
| | | | | RCV000021933 | |
| BTD | 3 | 15686693 | 15686693 | RCV000001977; RCV000078064; | SNV |
| | | | | RCV000001981; RCV000021912; | |
| | | | | RCV000021936; RCV000021952; | |
| | | | | RCV000021933 | |
| BTD | 3 | 15686693 | 15686693 | RCV000001977; RCV000078064; | SNV |
| | | | | RCV000001981; RCV000021912; | |
| | | | | RCV000021936; RCV000021952; | |
| | | | | RCV000021933 | |
| BTD | 3 | 15686693 | 15686693 | RCV000001977; RCV000078064; | SNV |
| | | | | RCV000001981; RCV000021912; | |
| | | | | RCV000021936; RCV000021952; | |
| | | | | RCV000021933 | |
| BTD | 3 | 15686693 | 15686693 | RCV000001977; RCV000078064; | SNV |
| | | | | RCV000001981; RCV000021912; | |
| | | | | RCV000021936; RCV000021952; | |
| | | | | RCV000021933 | |
| BTD | 3 | 15686696 | 15686696 | RCV000022006 | SNV |
| BTD | 3 | 15686697 | 15686697 | RCV000022007; RCV000021985 | SNV |
| BTD | 3 | 15686697 | 15686697 | RCV000022007; RCV000021985 | SNV |
| BTD | 3 | 15686702 | 15686702 | RCV000022008 | SNV |
| BTD | 3 | 15686715 | 15686715 | RCV000022009 | SNV |
| BTD | 3 | 15686724 | 15686724 | RCV000021903 | SNV |
| BTD | 3 | 15686731 | 15686731 | RCV000001979; RCV000078065 | SNV |
| BTD | 3 | 15686732 | 15686732 | RCV000022010 | SNV |
| BTD | 3 | 15686747 | 15686747 | RCV000022011 | deletion |
| BTD | 3 | 15686751 | 15686751 | RCV000022012 | SNV |
| BTD | 3 | 15686757 | 15686757 | RCV000032023 | duplication |
| BTD | 3 | 15686795 | 15686795 | RCV000022014 | SNV |
| BTD | 3 | 15686795 | 15686795 | RCV000022034 | SNV |
| BTD | 3 | 15686818 | 15686818 | RCV000022015 | SNV |
| BTD | 3 | 15686822 | 15686822 | RCV000022016 | SNV |
| BTD | 3 | 15686822 | 15686822 | RCV000022017 | deletion |
| BTD | 3 | 15686826 | 15686826 | RCV000022018 | SNV |
| BTD | 3 | 15686829 | 15686829 | RCV000001980 | SNV |
| BTD | 3 | 15686852 | 15686852 | RCV000022019; RCV000078068 | SNV |
| BTD | 3 | 15686856 | 15686856 | RCV000022020 | duplication |
| BTD | 3 | 15686871 | 15686875 | RCV000078069 | deletion |
| BTD | 3 | 15686874 | 15686874 | RCV000022021 | SNV |
| BTD | 3 | 15686894 | 15686894 | RCV000022022 | SNV |
| BTD | 3 | 15686958 | 15686958 | RCV000001974 | SNV |
| BTD | 3 | 15686973 | 15686973 | RCV000022023 | SNV |
| BTD | 3 | 15686975 | 15686975 | RCV000001975; RCV000078070 | SNV |
| BTD | 3 | 15686976 | 15686976 | RCV000022024 | SNV |
| BTD | 3 | 15686979 | 15686979 | RCV000022025 | duplication |
| BTD | 3 | 15686982 | 15686982 | RCV000022026 | SNV |
| BTD | 3 | 15686990 | 15686990 | RCV000022027 | SNV |
| BTD | 3 | 15686992 | 15686992 | RCV000022028; RCV000078071 | SNV |
| CBS | 21 | 44474030 | 44474030 | RCV000000146 | SNV |
| CBS | 21 | 44478325 | 44478325 | RCV000000153 | SNV |
| CBS | 21 | 44478972 | 44478972 | RCV000000149; RCV000000148; | SNV |
| | | | | RCV000078108 | |
| CBS | 21 | 44479037 | 44479037 | RCV000000152 | SNV |
| CBS | 21 | 44479409 | 44479409 | RCV000000145 | SNV |
| CBS | 21 | 44480638 | 44480638 | RCV000000154; RCV000078106 | SNV |
| CBS | 21 | 44482454 | 44482454 | RCV000078105 | SNV |
| CBS | 21 | 44483098 | 44483098 | RCV000000137; RCV000000138; | SNV |
| | | | | RCV000078112 | |
| CBS | 21 | 44483184 | 44483184 | RCV000000142; RCV000000141; | SNV |
| | | | | RCV000078111 | |
| CBS | 21 | 44484041 | 44484041 | RCV000000147 | SNV |
| CBS | 21 | 44485591 | 44485591 | RCV000000155 | SNV |
| CBS | 21 | 44485755 | 44485755 | RCV000000150 | SNV |
| CBS | 21 | 44486370 | 44486370 | RCV000000139 | SNV |
| CBS | 21 | 44486374 | 44486374 | RCV000000144 | SNV |
| CBS | 21 | 44486389 | 44486389 | RCV000000143 | SNV |
| CBS | 21 | 44486463 | 44486463 | RCV000000140 | SNV |
| CFTR | 7 | 117120149 | 117120149 | RCV000056356 | SNV |
| CFTR | 7 | 117120162 | 117120162 | RCV000029477 | SNV |
| CFTR | 7 | 117120167 | 117120167 | RCV000007657 | SNV |
| CFTR | 7 | 117144306 | 117144417 | RCV000007648 | deletion |
| CFTR | 7 | 117144367 | 117144367 | RCV000029470 | SNV |
| CFTR | 7 | 117144368 | 117144368 | RCV000056342 | SNV |
| CFTR | 7 | 117144378 | 117144378 | RCV000029472 | SNV |
| CFTR | 7 | 117149087 | 117149196 | RCV000007648 | deletion |
| CFTR | 7 | 117149094 | 117149094 | RCV000007599 | SNV |
| CFTR | 7 | 117149101 | 117149101 | RCV000056355 | SNV |
| CFTR | 7 | 117149123 | 117149123 | RCV000056357 | SNV |
| CFTR | 7 | 117149146 | 117149146 | RCV000056362 | SNV |
| CFTR | 7 | 117149147 | 117149147 | RCV000029494; RCV000119039; | SNV |
| | | | | RCV000078985; RCV000116686 | |
| CFTR | 7 | 117149177 | 117149177 | RCV000007563 | SNV |
| CFTR | 7 | 117149185 | 117149186 | RCV000007654 | deletion |
| CFTR | 7 | 117149194 | 117149194 | RCV000007603 | SNV |
| CFTR | 7 | 117170952 | 117170952 | RCV000056370 | SNV |
| CFTR | 7 | 117170953 | 117170953 | RCV000007601 | SNV |
| CFTR | 7 | 117170953 | 117170953 | RCV000007606 | SNV |
| CFTR | 7 | 117170971 | 117170971 | RCV000056375 | SNV |
| CFTR | 7 | 117170992 | 117170992 | RCV000046792 | deletion |
| CFTR | 7 | 117171004 | 117171006 | RCV000056378 | indel |
| CFTR | 7 | 117171005 | 117171005 | RCV000007618 | SNV |
| CFTR | 7 | 117171007 | 117171007 | RCV000007527; RCV000058930 | SNV |
| CFTR | 7 | 117171028 | 117171028 | RCV000056382 | SNV |
| CFTR | 7 | 117171029 | 117171029 | RCV000007528; RCV000007529; | SNV |
| | | | | RCV000078997 | |
| CFTR | 7 | 117171034 | 117171034 | RCV000029525 | SNV |
| CFTR | 7 | 117171045 | 117171045 | RCV000056385 | SNV |
| CFTR | 7 | 117171049 | 117171049 | RCV000029528 | SNV |
| CFTR | 7 | 117171061 | 117171062 | RCV000029532 | duplication |
| CFTR | 7 | 117171103 | 117171103 | RCV000007566 | deletion |
| CFTR | 7 | 117171108 | 117171108 | RCV000007567 | deletion |
| CFTR | 7 | 117171121 | 117171121 | RCV000056392 | deletion |
| CFTR | 7 | 117171138 | 117171155 | RCV000007608 | deletion |
| CFTR | 7 | 117174371 | 117174371 | RCV000056393 | deletion |
| CFTR | 7 | 117174372 | 117174372 | RCV000056394 | SNV |
| CFTR | 7 | 117174417 | 117174417 | RCV000047199; RCV000079010 | SNV |
| CFTR | 7 | 117175301 | 117175301 | RCV000056398 | SNV |
| CFTR | 7 | 117175314 | 117175314 | RCV000029542 | SNV |
| CFTR | 7 | 117175317 | 117175317 | RCV000056399 | SNV |
| CFTR | 7 | 117175335 | 117175335 | RCV000056400 | SNV |
| CFTR | 7 | 117175339 | 117175339 | RCV000007611; RCV000079011 | SNV |
| CFTR | 7 | 117175372 | 117175372 | RCV000007660 | SNV |
| CFTR | 7 | 117175380 | 117175380 | RCV000056401 | SNV |
| CFTR | 7 | 117175442 | 117175463 | RCV000007565 | deletion |
| CFTR | 7 | 117176661 | 117176661 | RCV000047258; RCV000079013 | deletion |
| CFTR | 7 | 117176663 | 117176664 | RCV000007627 | deletion |
| CFTR | 7 | 117176683 | 117176683 | RCV000029545 | SNV |
| CFTR | 7 | 117176704 | 117176704 | RCV000029546 | SNV |
| CFTR | 7 | 117176711 | 117176711 | RCV000029547 | SNV |
| CFTR | 7 | 117176718 | 117176719 | RCV000007658 | duplication |
| CFTR | 7 | 117180217 | 117180217 | RCV000007573 | SNV |
| CFTR | 7 | 117180219 | 117180221 | RCV000047290; RCV000079015 | deletion |
| CFTR | 7 | 117180232 | 117180232 | RCV000007583; RCV000079016 | deletion |
| CFTR | 7 | 117180242 | 117180242 | RCV000029549 | SNV |
| CFTR | 7 | 117180272 | 117180272 | RCV000056402 | SNV |
| CFTR | 7 | 117180281 | 117180281 | RCV000029550 | SNV |
| CFTR | 7 | 117180284 | 117180284 | RCV000007559 | SNV |
| CFTR | 7 | 117180291 | 117180291 | RCV000056337 | SNV |
| CFTR | 7 | 117180297 | 117180297 | RCV000007614 | SNV |
| CFTR | 7 | 117180305 | 117180305 | RCV000056338 | SNV |
| CFTR | 7 | 117180306 | 117180307 | RCV000007554 | duplication |
| CFTR | 7 | 117180306 | 117180307 | RCV000056339 | insertion |
| CFTR | 7 | 117180324 | 117180324 | RCV000007530 | SNV |
| CFTR | 7 | 117180324 | 117180324 | RCV000007591 | SNV |
| CFTR | 7 | 117180324 | 117180324 | RCV000007602; RCV000029468 | SNV |
| CFTR | 7 | 117180330 | 117180330 | RCV000007592 | SNV |
| CFTR | 7 | 117180338 | 117180338 | RCV000029469 | SNV |
| CFTR | 7 | 117180339 | 117180339 | RCV000007619 | SNV |
| CFTR | 7 | 117180359 | 117180359 | RCV000007589 | SNV |
| CFTR | 7 | 117180363 | 117180363 | RCV000007589 | SNV |
| CFTR | 7 | 117180365 | 117180365 | RCV000007555 | deletion |
| CFTR | 7 | 117180367 | 117180367 | RCV000007634 | deletion |
| CFTR | 7 | 117180374 | 117180374 | RCV000046229 | SNV |
| CFTR | 7 | 117180377 | 117180378 | RCV000007574 | deletion |
| CFTR | 7 | 117182080 | 117182081 | RCV000056341 | duplication |
| CFTR | 7 | 117182155 | 117182155 | RCV000056343 | SNV |
| CFTR | 7 | 117182156 | 117182156 | RCV000056344 | SNV |
| CFTR | 7 | 117188812 | 117188812 | RCV000046274; RCV000078976 | SNV |
| CFTR | 7 | 117188814 | 117188815 | RCV000056346 | insertion |
| CFTR | 7 | 117188849 | 117188849 | RCV000007531 | SNV |
| CFTR | 7 | 117188852 | 117188852 | RCV000029474 | SNV |
| CFTR | 7 | 117188858 | 117188858 | RCV000007552 | SNV |
| CFTR | 7 | 117199517 | 117199517 | RCV000056347 | SNV |
| CFTR | 7 | 117199522 | 117199522 | RCV000029475 | SNV |
| CFTR | 7 | 117199522 | 117199522 | RCV000056348 | SNV |
| CFTR | 7 | 117199525 | 117199525 | RCV000029476 | SNV |
| CFTR | 7 | 117199563 | 117199563 | RCV000007607; RCV000078977 | SNV |
| CFTR | 7 | 117199591 | 117199591 | RCV000056349 | SNV |
| CFTR | 7 | 117199600 | 117199600 | RCV000007575 | SNV |
| CFTR | 7 | 117199602 | 117199602 | RCV000007526 | SNV |
| CFTR | 7 | 117199602 | 117199603 | RCV000007640 | deletion |
| CFTR | 7 | 117199644 | 117199646 | RCV000007525 | deletion |
| CFTR | 7 | 117199646 | 117199648 | RCV000007523; RCV000007524; | deletion |
| | | | | RCV000119038; RCV000058929 | |
| CFTR | 7 | 117199670 | 117199671 | RCV000007560 | deletion |
| CFTR | 7 | 117199683 | 117199683 | RCV000007570 | SNV |
| CFTR | 7 | 117199683 | 117199683 | RCV000046339; RCV000029478; | SNV |
| | | | | RCV000078979 | |
| CFTR | 7 | 117199697 | 117199697 | RCV000007571 | SNV |
| CFTR | 7 | 117227792 | 117227792 | RCV000007532 | SNV |
| CFTR | 7 | 117227809 | 117227809 | RCV000007593 | SNV |
| CFTR | 7 | 117227832 | 117227832 | RCV000007535; RCV000119041; | SNV |
| | | | | RCV000058931 | |
| CFTR | 7 | 117227853 | 117227853 | RCV000043664; RCV000007538 | SNV |
| CFTR | 7 | 117227853 | 117227853 | RCV000043664; RCV000007538 | SNV |
| CFTR | 7 | 117227854 | 117227854 | RCV000007536 | SNV |
| CFTR | 7 | 117227854 | 117227854 | RCV000007537 | SNV |
| CFTR | 7 | 117227855 | 117227855 | RCV000056350; RCV000007538 | SNV |
| CFTR | 7 | 117227855 | 117227855 | RCV000056350; RCV000007538 | SNV |
| CFTR | 7 | 117227859 | 117227859 | RCV000007562 | SNV |
| CFTR | 7 | 117227860 | 117227860 | RCV000007540; RCV000119250; | SNV |
| | | | | RCV000119040 | |
| CFTR | 7 | 117227862 | 117227862 | RCV000007622 | SNV |
| CFTR | 7 | 117227865 | 117227865 | RCV000007542 | SNV |
| CFTR | 7 | 117227866 | 117227866 | RCV000007646 | SNV |
| CFTR | 7 | 117227874 | 117227874 | RCV000007617; RCV000029482 | SNV |
| CFTR | 7 | 117227881 | 117227881 | RCV000029483 | SNV |
| CFTR | 7 | 117227883 | 117227883 | RCV000007543 | SNV |
| CFTR | 7 | 117227887 | 117227887 | RCV000007533 | SNV |
| CFTR | 7 | 117227887 | 117227887 | RCV000007576 | SNV |
| CFTR | 7 | 117230406 | 117230406 | RCV000056352 | SNV |
| CFTR | 7 | 117230409 | 117230409 | RCV000007662 | SNV |
| CFTR | 7 | 117230414 | 117230414 | RCV000007534 | SNV |
| CFTR | 7 | 117230419 | 117230419 | RCV000029485 | deletion |
| CFTR | 7 | 117230448 | 117230448 | RCV000007539 | SNV |
| CFTR | 7 | 117230454 | 117230454 | RCV000029486; RCV000007585; | SNV |
| | | | | RCV000078981 | |
| CFTR | 7 | 117230458 | 117230458 | RCV000029487 | SNV |
| CFTR | 7 | 117230480 | 117230480 | RCV000056353 | SNV |
| CFTR | 7 | 117232038 | 117232121 | RCV000007568 | deletion |
| CFTR | 7 | 117232074 | 117232074 | RCV000046494; RCV000078982 | SNV |
| CFTR | 7 | 117232086 | 117232086 | RCV000046498; RCV000029488 | SNV |
| CFTR | 7 | 117232132 | 117232132 | RCV000029489 | deletion |
| CFTR | 7 | 117232144 | 117232152 | RCV000046508 | indel |
| CFTR | 7 | 117232164 | 117232164 | RCV000007623 | SNV |
| CFTR | 7 | 117232233 | 117232233 | RCV000029491 | deletion |
| CFTR | 7 | 117232272 | 117232273 | RCV000029492 | indel |
| CFTR | 7 | 117232273 | 117232273 | RCV000043563 | deletion |
| CFTR | 7 | 117232273 | 117232274 | RCV000029493 | duplication |
| CFTR | 7 | 117232346 | 117232346 | RCV000056359 | SNV |
| CFTR | 7 | 117232349 | 117232349 | RCV000007624 | SNV |
| CFTR | 7 | 117232367 | 117232367 | RCV000007594 | SNV |
| CFTR | 7 | 117232396 | 117232397 | RCV000007605 | duplication |
| CFTR | 7 | 117232416 | 117232416 | RCV000056360 | SNV |
| CFTR | 7 | 117232436 | 117232436 | RCV000056361 | deletion |
| CFTR | 7 | 117232511 | 117232511 | RCV000056363 | SNV |
| CFTR | 7 | 117232512 | 117232512 | RCV000007641 | deletion |
| CFTR | 7 | 117232595 | 117232595 | RCV000046585; RCV000029495 | SNV |
| CFTR | 7 | 117232643 | 117232644 | RCV000007541 | duplication |
| CFTR | 7 | 117232674 | 117232674 | RCV000056364 | deletion |
| CFTR | 7 | 117232685 | 117232685 | RCV000056365 | SNV |
| CFTR | 7 | 117232700 | 117232700 | RCV000007577 | SNV |
| CFTR | 7 | 117234984 | 117234984 | RCV000056366 | SNV |
| CFTR | 7 | 117235030 | 117235030 | RCV000056367 | SNV |
| CFTR | 7 | 117235031 | 117235031 | RCV000007547 | SNV |
| CFTR | 7 | 117235044 | 117235044 | RCV000007561 | SNV |
| CFTR | 7 | 117235076 | 117235076 | RCV000056368 | deletion |
| CFTR | 7 | 117235090 | 117235090 | RCV000029498 | SNV |
| CFTR | 7 | 117243596 | 117243596 | RCV000007625 | SNV |
| CFTR | 7 | 117243663 | 117243663 | RCV000007626; RCV000007661 | SNV |
| CFTR | 7 | 117243663 | 117243663 | RCV000007626; RCV000007661 | SNV |
| CFTR | 7 | 117243665 | 117243666 | RCV000007569 | insertion |
| CFTR | 7 | 117243666 | 117243666 | RCV000007548 | SNV |
| CFTR | 7 | 117243696 | 117243696 | RCV000029503 | SNV |
| CFTR | 7 | 117243708 | 117243708 | RCV000056371 | SNV |
| CFTR | 7 | 117243738 | 117243739 | RCV000029504 | duplication |
| CFTR | 7 | 117243741 | 117243741 | RCV000046693; RCV000029505 | SNV |
| CFTR | 7 | 117243762 | 117243762 | RCV000056372 | SNV |
| CFTR | 7 | 117243773 | 117243773 | RCV000007628 | SNV |
| CFTR | 7 | 117243803 | 117243803 | RCV000056373 | deletion |
| CFTR | 7 | 117243836 | 117243836 | RCV000056374 | SNV |
| CFTR | 7 | 117246728 | 117246728 | RCV000046718; RCV000029509 | SNV |
| CFTR | 7 | 117246751 | 117246751 | RCV000029510 | SNV |
| CFTR | 7 | 117246807 | 117246807 | RCV000029512; RCV000078990 | SNV |
| CFTR | 7 | 117250572 | 117250572 | RCV000056376 | SNV |
| CFTR | 7 | 117250575 | 117250575 | RCV000046745; RCV000007650; | SNV |
| | | | | RCV000007651; RCV000078991 | |
| CFTR | 7 | 117250622 | 117250622 | RCV000029514 | SNV |
| CFTR | 7 | 117250651 | 117250656 | RCV000046775; RCV000078992 | deletion |
| CFTR | 7 | 117250679 | 117250679 | RCV000029516 | SNV |
| CFTR | 7 | 117251649 | 117251649 | RCV000046799; RCV000029520 | SNV |
| CFTR | 7 | 117251656 | 117251656 | RCV000007642 | deletion |
| CFTR | 7 | 117251683 | 117251684 | RCV000007581 | duplication |
| CFTR | 7 | 117251689 | 117251689 | RCV000007629 | SNV |
| CFTR | 7 | 117251691 | 117251691 | RCV000007582 | SNV |
| CFTR | 7 | 117251692 | 117251692 | RCV000007578 | SNV |
| CFTR | 7 | 117251694 | 117251694 | RCV000007579; RCV000078995 | SNV |
| CFTR | 7 | 117251704 | 117251704 | RCV000046825; RCV000029521 | SNV |
| CFTR | 7 | 117251707 | 117251707 | RCV000007630 | SNV |
| CFTR | 7 | 117251725 | 117251725 | RCV000056377 | SNV |
| CFTR | 7 | 117251749 | 117251749 | RCV000007631 | SNV |
| CFTR | 7 | 117251761 | 117251761 | RCV000007615 | SNV |
| CFTR | 7 | 117251771 | 117251771 | RCV000056379; RCV000119251 | SNV |
| CFTR | 7 | 117251771 | 117251771 | RCV000056379; RCV000119251 | SNV |
| CFTR | 7 | 117251771 | 117251771 | RCV000056380; RCV000119251 | SNV |
| CFTR | 7 | 117251771 | 117251771 | RCV000056380; RCV000119251 | SNV |
| CFTR | 7 | 117251797 | 117251797 | RCV000032712; RCV000078996 | SNV |
| CFTR | 7 | 117251805 | 117251805 | RCV000056381 | SNV |
| CFTR | 7 | 117254753 | 117254753 | RCV000046895; RCV000029522 | SNV |
| CFTR | 7 | 117267579 | 117267579 | RCV000007564 | SNV |
| CFTR | 7 | 117267591 | 117267591 | RCV000007557 | SNV |
| CFTR | 7 | 117267599 | 117267600 | RCV000007652 | duplication |
| CFTR | 7 | 117267635 | 117267635 | RCV000029523 | deletion |
| CFTR | 7 | 117267642 | 117267643 | RCV000007643 | insertion |
| CFTR | 7 | 117267694 | 117267694 | RCV000056383 | SNV |
| CFTR | 7 | 117267718 | 117267718 | RCV000007633 | SNV |
| CFTR | 7 | 117267719 | 117267719 | RCV000056384; RCV000078999 | SNV |
| CFTR | 7 | 117267766 | 117267766 | RCV000007544 | deletion |
| CFTR | 7 | 117267766 | 117267766 | RCV000007635 | SNV |
| CFTR | 7 | 117267798 | 117267798 | RCV000029526 | deletion |
| CFTR | 7 | 117267807 | 117267807 | RCV000007636 | SNV |
| CFTR | 7 | 117267819 | 117267819 | RCV000007596 | SNV |
| CFTR | 7 | 117282491 | 117282491 | RCV000007597 | SNV |
| CFTR | 7 | 117282505 | 117282505 | RCV000007661; RCV000046963 | SNV |
| CFTR | 7 | 117282505 | 117282505 | RCV000007661; RCV000046963 | SNV |
| CFTR | 7 | 117282505 | 117282505 | RCV000056386 | SNV |
| CFTR | 7 | 117282518 | 117282518 | RCV000007653 | deletion |
| CFTR | 7 | 117282520 | 117282520 | RCV000007637 | SNV |
| CFTR | 7 | 117282526 | 117282526 | RCV000007638 | SNV |
| CFTR | 7 | 117282537 | 117282537 | RCV000007613 | SNV |
| CFTR | 7 | 117282538 | 117282538 | RCV000007545 | SNV |
| CFTR | 7 | 117282542 | 117282542 | RCV000007598 | duplication |
| CFTR | 7 | 117282547 | 117282547 | RCV000046980 | duplication |
| CFTR | 7 | 117282582 | 117282582 | RCV000046985; RCV000007584 | SNV |
| CFTR | 7 | 117282620 | 117282620 | RCV000007549 | SNV |
| CFTR | 7 | 117282622 | 117282622 | RCV000007587 | SNV |
| CFTR | 7 | 117282631 | 117282631 | RCV000007604 | SNV |
| CFTR | 7 | 117282647 | 117282647 | RCV000007572 | SNV |
| CFTR | 7 | 117292900 | 117292903 | RCV000007616 | deletion |
| CFTR | 7 | 117292906 | 117292907 | RCV000056388 | duplication |
| CFTR | 7 | 117292915 | 117292915 | RCV000029533 | SNV |
| CFTR | 7 | 117292929 | 117292929 | RCV000007639 | SNV |
| CFTR | 7 | 117292931 | 117292931 | RCV000007556 | SNV |
| CFTR | 7 | 117292959 | 117292959 | RCV000007600 | SNV |
| CFTR | 7 | 117292969 | 117292969 | RCV000007553 | SNV |
| CFTR | 7 | 117292979 | 117292980 | RCV000029534 | insertion |
| CFTR | 7 | 117304775 | 117304775 | RCV000029535 | SNV |
| CFTR | 7 | 117304824 | 117304824 | RCV000029536 | SNV |
| CFTR | 7 | 117304834 | 117304834 | RCV000007659 | SNV |
| CFTR | 7 | 117304834 | 117304834 | RCV000029537 | SNV |
| CFTR | 7 | 117304855 | 117304858 | RCV000056390 | indel |
| CFTR | 7 | 117306970 | 117306970 | RCV000056391 | deletion |
| CFTR | 7 | 117306999 | 117306999 | RCV000029541 | SNV |
| CFTR | 7 | 117307083 | 117307083 | RCV000047130; RCV000007644 | SNV |
| CFTR | 7 | 117307145 | 117307145 | RCV000047135; RCV000079008 | SNV |
| CPT1A | 11 | 68527116 | 68527116 | RCV000055865 | SNV |
| CPT1A | 11 | 68527706 | 68527706 | RCV000009638 | SNV |
| CPT1A | 11 | 68527709 | 68527709 | RCV000009636 | SNV |
| CPT1A | 11 | 68527728 | 68527806 | RCV000009635 | deletion |
| CPT1A | 11 | 68529002 | 68529155 | RCV000009635 | deletion |
| CPT1A | 11 | 68529002 | 68529004 | RCV000055864 | deletion |
| CPT1A | 11 | 68530094 | 68530226 | RCV000009635 | deletion |
| CPT1A | 11 | 68540736 | 68540736 | RCV000055862 | SNV |
| CPT1A | 11 | 68540873 | 68540873 | RCV000055861 | deletion |
| CPT1A | 11 | 68542865 | 68542865 | RCV000055859 | SNV |
| CPT1A | 11 | 68542865 | 68542865 | RCV000055860 | SNV |
| CPT1A | 11 | 68542866 | 68542866 | RCV000009633 | SNV |
| CPT1A | 11 | 68548115 | 68548115 | RCV000055858 | SNV |
| CPT1A | 11 | 68548130 | 68548130 | RCV000055857; RCV000079911 | SNV |
| CPT1A | 11 | 68548141 | 68548141 | RCV000055856 | SNV |
| CPT1A | 11 | 68548173 | 68548173 | RCV000055855 | SNV |
| CPT1A | 11 | 68548205 | 68548205 | RCV000009628 | SNV |
| CPT1A | 11 | 68549252 | 68549252 | RCV000055854 | SNV |
| CPT1A | 11 | 68549350 | 68549350 | RCV000009632 | SNV |
| CPT1A | 11 | 68552367 | 68552367 | RCV000009629 | SNV |
| CPT1A | 11 | 68552377 | 68552377 | RCV000055853 | SNV |
| CPT1A | 11 | 68552419 | 68552419 | RCV000055852 | SNV |
| CPT1A | 11 | 68560802 | 68560802 | RCV000009637 | deletion |
| CPT1A | 11 | 68560804 | 68560804 | RCV000055872 | SNV |
| CPT1A | 11 | 68560809 | 68560809 | RCV000055870 | SNV |
| CPT1A | 11 | 68560838 | 68560838 | RCV000055869 | SNV |
| CPT1A | 11 | 68562328 | 68562328 | RCV000055868; RCV000124597 | SNV |
| CPT1A | 11 | 68571545 | 68571545 | RCV000055867 | SNV |
| CPT1A | 11 | 68575021 | 68575021 | RCV000055866; RCV000079915 | SNV |
| CPT1A | 11 | 68575090 | 68575090 | RCV000009630; RCV000079914 | SNV |
| CPT1A | 11 | 68579904 | 68579904 | RCV000079913 | SNV |
| CPT1A | 11 | 68579964 | 68579964 | RCV000079912 | SNV |
| CPT1A | 11 | 68582847 | 68582847 | RCV000055874 | SNV |
| CPT2 | 1 | 53662764 | 53662764 | RCV000009512; RCV000009511 | SNV |
| CPT2 | 1 | 53668099 | 53668099 | RCV000009510; RCV000078121 | SNV |
| CPT2 | 1 | 53675705 | 53675705 | RCV000009530 | SNV |
| CPT2 | 1 | 53675866 | 53675866 | RCV000009516 | SNV |
| CPT2 | 1 | 53675984 | 53675984 | RCV000009529 | SNV |
| CPT2 | 1 | 53676026 | 53676026 | RCV000009526 | SNV |
| CPT2 | 1 | 53676494 | 53676494 | RCV000009517; RCV000009518 | SNV |
| CPT2 | 1 | 53676584 | 53676585 | RCV000009520 | deletion |
| CPT2 | 1 | 53676585 | 53676586 | RCV000078117 | deletion |
| CPT2 | 1 | 53676688 | 53676688 | RCV000009520 | SNV |
| CPT2 | 1 | 53676706 | 53676706 | RCV000009528 | SNV |
| CPT2 | 1 | 53676853 | 53676853 | RCV000009519 | SNV |
| CPT2 | 1 | 53678947 | 53678947 | RCV000009513 | SNV |
| CPT2 | 1 | 53679173 | 53679173 | RCV000009514; RCV000009515 | SNV |
| CPT2 | 1 | 53679181 | 53679181 | RCV000009508; RCV000009509 | SNV |
| CYP21A2 | 6 | 32006291 | 32006291 | RCV000012938 | SNV |
| CYP21A2 | 6 | 32006910 | 32006917 | RCV000012946 | deletion |
| CYP21A2 | 6 | 32006939 | 32006939 | RCV000012966 | SNV |
| CYP21A2 | 6 | 32007203 | 32007203 | RCV000012933 | SNV |
| CYP21A2 | 6 | 32007584 | 32007584 | RCV000012947 | SNV |
| CYP21A2 | 6 | 32007587 | 32007587 | RCV000012947; RCV000055823 | SNV |
| CYP21A2 | 6 | 32007587 | 32007587 | RCV000012947; RCV000055823 | SNV |
| CYP21A2 | 6 | 32007593 | 32007593 | RCV000012947; RCV000055823 | SNV |
| CYP21A2 | 6 | 32007593 | 32007593 | RCV000012947; RCV000055823 | SNV |
| CYP21A2 | 6 | 32007887 | 32007887 | RCV000012935; RCV000012936; | SNV |
| | | | | RCV000012934 | |
| CYP21A2 | 6 | 32007887 | 32007887 | RCV000055820 | SNV |
| CYP21A2 | 6 | 32007917 | 32007917 | RCV000012940 | SNV |
| CYP21A2 | 6 | 32007963 | 32007964 | RCV000012964 | duplication |
| CYP21A2 | 6 | 32007966 | 32007966 | RCV000055821 | duplication |
| CYP21A2 | 6 | 32008198 | 32008198 | RCV000012951 | SNV |
| CYP21A2 | 6 | 32008312 | 32008312 | RCV000012937 | SNV |
| CYP21A2 | 6 | 32008543 | 32008543 | RCV000012953 | SNV |
| CYP21A2 | 6 | 32008783 | 32008783 | RCV000012943 | SNV |
| CYP21A2 | 6 | 32008874 | 32008875 | RCV000012941 | indel |
| DBT | 1 | 100661812 | 100661812 | RCV000012725 | SNV |
| DBT | 1 | 100661813 | 100661813 | RCV000079937 | SNV |
| DBT | 1 | 100661905 | 100661905 | RCV000012730 | SNV |
| DBT | 1 | 100661969 | 100661969 | RCV000079933 | SNV |
| DBT | 1 | 100672060 | 100672060 | RCV000012727 | SNV |
| DBT | 1 | 100680373 | 100680373 | RCV000079960 | SNV |
| DBT | 1 | 100680411 | 100680411 | RCV000079959 | SNV |
| DBT | 1 | 100680441 | 100680441 | RCV000079958 | SNV |
| DBT | 1 | 100680485 | 100680485 | RCV000012721; RCV000079957 | SNV |
| DBT | 1 | 100681641 | 100681641 | RCV000079951 | SNV |
| DBT | 1 | 100681730 | 100681730 | RCV000012731 | SNV |
| DBT | 1 | 100696362 | 100696362 | RCV000079944 | deletion |
| DBT | 1 | 100696377 | 100696383 | RCV000079943 | deletion |
| DBT | 1 | 100696428 | 100696428 | RCV000012726 | SNV |
| DBT | 1 | 100696447 | 100696450 | RCV000079942 | deletion |
| DBT | 1 | 100700992 | 100700992 | RCV000079941 | SNV |
| DBT | 1 | 100715325 | 100715325 | RCV000079945 | SNV |
| DLD | 7 | 107542204 | 107542204 | RCV000033218 | SNV |
| DLD | 7 | 107542785 | 107542785 | RCV000012742 | SNV |
| DLD | 7 | 107555951 | 107555951 | RCV000012744 | SNV |
| DLD | 7 | 107557752 | 107557752 | RCV000012752 | SNV |
| DLD | 7 | 107557794 | 107557794 | RCV000012751 | SNV |
| DLD | 7 | 107557849 | 107557849 | RCV000012747 | SNV |
| DLD | 7 | 107559516 | 107559516 | RCV000033216 | SNV |
| DLD | 7 | 107559524 | 107559524 | RCV000033217 | SNV |
| DLD | 7 | 107559543 | 107559543 | RCV000012743 | SNV |
| DLD | 7 | 107559657 | 107559657 | RCV000012746 | SNV |
| DNAJC19 | 3 | 180702479 | 180702479 | RCV000106304 | deletion |
| DUOX2 | 15 | 45396374 | 45396374 | RCV000004279 | SNV |
| DUOX2 | 15 | 45398415 | 45398415 | RCV000004278 | SNV |
| DUOX2 | 15 | 45401085 | 45401085 | RCV000004277 | SNV |
| DUOX2 | 15 | 45402093 | 45402093 | RCV000004280 | SNV |
| ETFA | 15 | 76566772 | 76566772 | RCV000002712 | SNV |
| ETFA | 15 | 76578804 | 76578804 | RCV000002711 | SNV |
| ETFA | 15 | 76584777 | 76584777 | RCV000002713 | SNV |
| ETFB | 19 | 51850260 | 51850260 | RCV000018200 | SNV |
| ETFB | 19 | 51853639 | 51853639 | RCV000018202 | SNV |
| ETFDH | 4 | 159593610 | 159593610 | RCV000012806 | SNV |
| ETFDH | 4 | 159603421 | 159603421 | RCV000012808 | SNV |
| ETFDH | 4 | 159603551 | 159603551 | RCV000012810 | SNV |
| ETFDH | 4 | 159606289 | 159606289 | RCV000012809 | SNV |
| ETFDH | 4 | 159606289 | 159606289 | RCV000024282 | SNV |
| ETFDH | 4 | 159624588 | 159624588 | RCV000024305 | SNV |
| ETFDH | 4 | 159624692 | 159624692 | RCV000081076 | SNV |
| ETFDH | 4 | 159627422 | 159627422 | RCV000081077 | SNV |
| ETFDH | 4 | 159627503 | 159627503 | RCV000024306 | SNV |
| ETFDH | 4 | 159629648 | 159629648 | RCV000081078 | deletion |
| FAH | 15 | 80445443 | 80445443 | RCV000012640 | SNV |
| FAH | 15 | 80450512 | 80450512 | RCV000020126 | SNV |
| FAH | 15 | 80454624 | 80454624 | RCV000012641 | SNV |
| FAH | 15 | 80460394 | 80460394 | RCV000078137 | SNV |
| FAH | 15 | 80460605 | 80460605 | RCV000012649; RCV000078138 | SNV |
| FAH | 15 | 80464582 | 80464582 | RCV000020128 | SNV |
| FAH | 15 | 80465431 | 80465431 | RCV000020129 | SNV |
| FAH | 15 | 80465435 | 80465435 | RCV000012648 | SNV |
| FAH | 15 | 80465485 | 80465485 | RCV000012650 | SNV |
| FAH | 15 | 80472514 | 80472514 | RCV000012644 | SNV |
| FAH | 15 | 80472526 | 80472526 | RCV000012643; RCV000020125 | SNV |
| FAH | 15 | 80473390 | 80473390 | RCV000012646 | SNV |
| FAH | 15 | 80473411 | 80473411 | RCV000012642 | SNV |
| FAH | 15 | 80473462 | 80473462 | RCV000012647 | SNV |
| GAA | 17 | 78078692 | 78078692 | RCV000078179 | SNV |
| GAA | 17 | 78078910 | 78078910 | RCV000004248; RCV000078181 | deletion |
| GAA | 17 | 78081373 | 78081373 | RCV000004250 | SNV |
| GAA | 17 | 78081617 | 78081617 | RCV000004251 | SNV |
| GAA | 17 | 78081636 | 78081636 | RCV000004240 | SNV |
| GAA | 17 | 78081693 | 78081693 | RCV000004236 | SNV |
| GAA | 17 | 78084553 | 78084553 | RCV000078159 | SNV |
| GAA | 17 | 78084749 | 78084749 | RCV000004237 | SNV |
| GAA | 17 | 78084773 | 78084774 | RCV000004241 | indel |
| GAA | 17 | 78084822 | 78084822 | RCV000004247 | SNV |
| GAA | 17 | 78086713 | 78086713 | RCV000004238 | SNV |
| GAA | 17 | 78086721 | 78086721 | RCV000004244; RCV000055768 | SNV |
| GAA | 17 | 78086798 | 78086798 | RCV000078163 | SNV |
| GAA | 17 | 78087042 | 78087046 | RCV000078166 | duplication |
| GAA | 17 | 78087081 | 78087081 | RCV000078167 | SNV |
| GAA | 17 | 78087149 | 78087149 | RCV000004239 | SNV |
| GAA | 17 | 78091992 | 78092156 | RCV000004246 | deletion |
| GAA | 17 | 78092022 | 78092022 | RCV000078174 | SNV |
| GAA | 17 | 78092054 | 78092054 | RCV000078175 | deletion |
| GAA | 17 | 78092070 | 78092070 | RCV000004249 | SNV |
| GAA | 17 | 78092512 | 78092514 | RCV000004243 | deletion |
| GALC | 14 | 88407777 | 88407777 | RCV000023589 | SNV |
| GALC | 14 | 88411937 | 88411937 | RCV000023588 | SNV |
| GALC | 14 | 88429736 | 88429736 | RCV000004022 | SNV |
| GALC | 14 | 88431929 | 88431929 | RCV000023594 | SNV |
| GALC | 14 | 88434725 | 88434725 | RCV000078208 | SNV |
| GALC | 14 | 88434730 | 88434730 | RCV000023593 | SNV |
| GALC | 14 | 88452941 | 88452941 | RCV000078200 | SNV |
| GALC | 14 | 88459388 | 88459388 | RCV000023595 | SNV |
| GALE | 1 | 24122673 | 24122673 | RCV000003866; RCV000078697 | SNV |
| GALE | 1 | 24122692 | 24122692 | RCV000003865 | SNV |
| GALE | 1 | 24122724 | 24122724 | RCV000020295 | SNV |
| GALE | 1 | 24123212 | 24123212 | RCV000003864; RCV000078693 | SNV |
| GALE | 1 | 24123267 | 24123267 | RCV000020294 | SNV |
| GALE | 1 | 24123618 | 24123618 | RCV000003860 | SNV |
| GALE | 1 | 24124208 | 24124208 | RCV000020293 | SNV |
| GALE | 1 | 24124650 | 24124650 | RCV000003863 | SNV |
| GALE | 1 | 24124678 | 24124678 | RCV000020292; RCV000003867 | SNV |
| GALE | 1 | 24124689 | 24124689 | RCV000003862 | SNV |
| GALE | 1 | 24125397 | 24125397 | RCV000003861 | SNV |
| GALK1 | 17 | 73754172 | 73754172 | RCV000005985 | SNV |
| GALK1 | 17 | 73759113 | 73759113 | RCV000005987 | SNV |
| GALK1 | 17 | 73760095 | 73760095 | RCV000005983 | SNV |
| GALK1 | 17 | 73761124 | 73761124 | RCV000005982 | SNV |
| GALK1 | 17 | 73761136 | 73761136 | RCV000005984 | SNV |
| GALT | 9 | 34646701 | 34646783 | RCV000114378 | deletion |
| GALT | 9 | 34646702 | 34646702 | RCV000022038 | SNV |
| GALT | 9 | 34646719 | 34646719 | RCV000022039 | deletion |
| GALT | 9 | 34646726 | 34646726 | RCV000022040 | SNV |
| GALT | 9 | 34646728 | 34646728 | RCV000022041 | SNV |
| GALT | 9 | 34646742 | 34646742 | RCV000022043; RCV000078221 | indel |
| GALT | 9 | 34646768 | 34646768 | RCV000022044 | SNV |
| GALT | 9 | 34646783 | 34646783 | RCV000022046 | SNV |
| GALT | 9 | 34646783 | 34646783 | RCV000022047 | SNV |
| GALT | 9 | 34646783 | 34646783 | RCV000031852 | SNV |
| GALT | 9 | 34647085 | 34647255 | RCV000114378 | deletion |
| GALT | 9 | 34647093 | 34647093 | RCV000022048 | SNV |
| GALT | 9 | 34647094 | 34647094 | RCV000022049 | SNV |
| GALT | 9 | 34647098 | 34647098 | RCV000022050 | SNV |
| GALT | 9 | 34647101 | 34647101 | RCV000022051 | SNV |
| GALT | 9 | 34647103 | 34647103 | RCV000022052 | SNV |
| GALT | 9 | 34647110 | 34647110 | RCV000022053 | SNV |
| GALT | 9 | 34647116 | 34647116 | RCV000022054 | SNV |
| GALT | 9 | 34647133 | 34647133 | RCV000003795 | SNV |
| GALT | 9 | 34647133 | 34647133 | RCV000022056 | SNV |
| GALT | 9 | 34647137 | 34647137 | RCV000022059; RCV000078214 | SNV |
| GALT | 9 | 34647137 | 34647141 | RCV000022058 | deletion |
| GALT | 9 | 34647155 | 34647155 | RCV000022060 | SNV |
| GALT | 9 | 34647155 | 34647155 | RCV000022061 | SNV |
| GALT | 9 | 34647163 | 34647163 | RCV000022062 | SNV |
| GALT | 9 | 34647166 | 34647166 | RCV000022063 | SNV |
| GALT | 9 | 34647200 | 34647200 | RCV000022065 | SNV |
| GALT | 9 | 34647200 | 34647200 | RCV000022066 | SNV |
| GALT | 9 | 34647202 | 34647202 | RCV000022067; RCV000078215 | SNV |
| GALT | 9 | 34647206 | 34647206 | RCV000029806 | SNV |
| GALT | 9 | 34647210 | 34647217 | RCV000022068 | deletion |
| GALT | 9 | 34647223 | 34647224 | RCV000022069 | insertion |
| GALT | 9 | 34647223 | 34647224 | RCV000032010 | deletion |
| GALT | 9 | 34647224 | 34647224 | RCV000003799 | SNV |
| GALT | 9 | 34647241 | 34647241 | RCV000022070 | SNV |
| GALT | 9 | 34647244 | 34647244 | RCV000022071 | SNV |
| GALT | 9 | 34647250 | 34647250 | RCV000022072 | SNV |
| GALT | 9 | 34647488 | 34647564 | RCV000114378 | deletion |
| GALT | 9 | 34647501 | 34647501 | RCV000022074 | SNV |
| GALT | 9 | 34647501 | 34647501 | RCV000022075 | SNV |
| GALT | 9 | 34647521 | 34647521 | RCV000022076 | SNV |
| GALT | 9 | 34647525 | 34647527 | RCV000078216 | deletion |
| GALT | 9 | 34647526 | 34647526 | RCV000022077; RCV000078217 | SNV |
| GALT | 9 | 34647528 | 34647528 | RCV000022079 | SNV |
| GALT | 9 | 34647528 | 34647528 | RCV000022080; RCV000078219 | SNV |
| GALT | 9 | 34647544 | 34647544 | RCV000022083 | SNV |
| GALT | 9 | 34647653 | 34647702 | RCV000114378 | deletion |
| GALT | 9 | 34647658 | 34647659 | RCV000022087 | duplication |
| GALT | 9 | 34647661 | 34647661 | RCV000022088 | SNV |
| GALT | 9 | 34647662 | 34647662 | RCV000022089 | SNV |
| GALT | 9 | 34647666 | 34647666 | RCV000022090 | SNV |
| GALT | 9 | 34647675 | 34647675 | RCV000022091 | SNV |
| GALT | 9 | 34647679 | 34647679 | RCV000022092 | SNV |
| GALT | 9 | 34647692 | 34647692 | RCV000022078 | SNV |
| GALT | 9 | 34647693 | 34647693 | RCV000022093 | SNV |
| GALT | 9 | 34647699 | 34647699 | RCV000022094 | SNV |
| GALT | 9 | 34647828 | 34647958 | RCV000114378 | deletion |
| GALT | 9 | 34647828 | 34647958 | RCV000022096 | deletion |
| GALT | 9 | 34647830 | 34647830 | RCV000022098 | SNV |
| GALT | 9 | 34647836 | 34647836 | RCV000029808 | SNV |
| GALT | 9 | 34647837 | 34647837 | RCV000022099 | SNV |
| GALT | 9 | 34647840 | 34647840 | RCV000022100 | SNV |
| GALT | 9 | 34647843 | 34647843 | RCV000022101 | SNV |
| GALT | 9 | 34647845 | 34647845 | RCV000022102 | SNV |
| GALT | 9 | 34647847 | 34647847 | RCV000022103 | SNV |
| GALT | 9 | 34647850 | 34647851 | RCV000032011 | duplication |
| GALT | 9 | 34647851 | 34647851 | RCV000022104 | deletion |
| GALT | 9 | 34647855 | 34647855 | RCV000003802; RCV000078220 | SNV |
| GALT | 9 | 34647855 | 34647855 | RCV000022105 | SNV |
| GALT | 9 | 34647861 | 34647861 | RCV000022106 | duplication |
| GALT | 9 | 34647864 | 34647864 | RCV000022107 | SNV |
| GALT | 9 | 34647867 | 34647867 | RCV000022108 | SNV |
| GALT | 9 | 34647875 | 34647875 | RCV000022109 | SNV |
| GALT | 9 | 34647876 | 34647876 | RCV000003793; RCV000078222 | SNV |
| GALT | 9 | 34647876 | 34647876 | RCV000022110 | SNV |
| GALT | 9 | 34647879 | 34647879 | RCV000022111 | SNV |
| GALT | 9 | 34647893 | 34647893 | RCV000022112 | SNV |
| GALT | 9 | 34647893 | 34647893 | RCV000022113; RCV000078223 | SNV |
| GALT | 9 | 34647894 | 34647894 | RCV000022114 | SNV |
| GALT | 9 | 34647899 | 34647899 | RCV000022115 | SNV |
| GALT | 9 | 34647903 | 34647903 | RCV000022116 | SNV |
| GALT | 9 | 34647911 | 34647911 | RCV000022117 | SNV |
| GALT | 9 | 34647911 | 34647911 | RCV000022118 | SNV |
| GALT | 9 | 34647913 | 34647913 | RCV000022119 | SNV |
| GALT | 9 | 34647933 | 34647933 | RCV000022120 | SNV |
| GALT | 9 | 34647941 | 34647941 | RCV000022121 | SNV |
| GALT | 9 | 34647947 | 34647947 | RCV000022123 | SNV |
| GALT | 9 | 34647950 | 34647950 | RCV000022124 | SNV |
| GALT | 9 | 34647953 | 34647953 | RCV000022125 | SNV |
| GALT | 9 | 34647956 | 34647956 | RCV000022126 | SNV |
| GALT | 9 | 34648111 | 34648168 | RCV000114378 | deletion |
| GALT | 9 | 34648111 | 34648168 | RCV000022096 | deletion |
| GALT | 9 | 34648111 | 34648111 | RCV000078224 | SNV |
| GALT | 9 | 34648113 | 34648113 | RCV000022135 | SNV |
| GALT | 9 | 34648113 | 34648113 | RCV000022136 | SNV |
| GALT | 9 | 34648116 | 34648116 | RCV000003800; RCV000078225 | SNV |
| GALT | 9 | 34648128 | 34648128 | RCV000022139 | SNV |
| GALT | 9 | 34648132 | 34648133 | RCV000022140 | insertion |
| GALT | 9 | 34648140 | 34648140 | RCV000022141 | SNV |
| GALT | 9 | 34648143 | 34648143 | RCV000022142 | SNV |
| GALT | 9 | 34648145 | 34648145 | RCV000022143 | SNV |
| GALT | 9 | 34648146 | 34648146 | RCV000022144 | SNV |
| GALT | 9 | 34648151 | 34648151 | RCV000003803 | SNV |
| GALT | 9 | 34648154 | 34648154 | RCV000022145 | SNV |
| GALT | 9 | 34648156 | 34648156 | RCV000022146 | SNV |
| GALT | 9 | 34648157 | 34648157 | RCV000022147 | SNV |
| GALT | 9 | 34648158 | 34648158 | RCV000022148 | SNV |
| GALT | 9 | 34648158 | 34648158 | RCV000022149 | SNV |
| GALT | 9 | 34648160 | 34648160 | RCV000022150 | SNV |
| GALT | 9 | 34648167 | 34648167 | RCV000003798; RCV000078227 | SNV |
| GALT | 9 | 34648330 | 34648453 | RCV000114378 | deletion |
| GALT | 9 | 34648330 | 34648453 | RCV000022096 | deletion |
| GALT | 9 | 34648331 | 34648344 | RCV000078228 | deletion |
| GALT | 9 | 34648334 | 34648334 | RCV000078229 | SNV |
| GALT | 9 | 34648340 | 34648340 | RCV000022155 | SNV |
| GALT | 9 | 34648341 | 34648341 | RCV000022156 | SNV |
| GALT | 9 | 34648346 | 34648346 | RCV000003808 | SNV |
| GALT | 9 | 34648350 | 34648350 | RCV000022158; RCV000078230 | SNV |
| GALT | 9 | 34648360 | 34648360 | RCV000022159 | SNV |
| GALT | 9 | 34648361 | 34648361 | RCV000022160 | SNV |
| GALT | 9 | 34648364 | 34648364 | RCV000022161 | deletion |
| GALT | 9 | 34648367 | 34648367 | RCV000022162 | SNV |
| GALT | 9 | 34648368 | 34648368 | RCV000022163 | SNV |
| GALT | 9 | 34648373 | 34648373 | RCV000003806 | SNV |
| GALT | 9 | 34648376 | 34648376 | RCV000022164 | SNV |
| GALT | 9 | 34648377 | 34648377 | RCV000022165 | SNV |
| GALT | 9 | 34648385 | 34648385 | RCV000022166 | SNV |
| GALT | 9 | 34648392 | 34648392 | RCV000022167; RCV000078231 | SNV |
| GALT | 9 | 34648392 | 34648392 | RCV000022168 | SNV |
| GALT | 9 | 34648400 | 34648400 | RCV000022170; RCV000078232 | SNV |
| GALT | 9 | 34648401 | 34648401 | RCV000022171 | SNV |
| GALT | 9 | 34648416 | 34648416 | RCV000022172 | SNV |
| GALT | 9 | 34648418 | 34648418 | RCV000022173 | deletion |
| GALT | 9 | 34648418 | 34648418 | RCV000022175 | SNV |
| GALT | 9 | 34648424 | 34648424 | RCV000022176 | SNV |
| GALT | 9 | 34648424 | 34648425 | RCV000022177 | duplication |
| GALT | 9 | 34648433 | 34648433 | RCV000022178 | SNV |
| GALT | 9 | 34648442 | 34648442 | RCV000022179 | SNV |
| GALT | 9 | 34648443 | 34648443 | RCV000022180 | SNV |
| GALT | 9 | 34648446 | 34648446 | RCV000022181 | SNV |
| GALT | 9 | 34648453 | 34648453 | RCV000022182 | SNV |
| GALT | 9 | 34648758 | 34648824 | RCV000114378 | deletion |
| GALT | 9 | 34648758 | 34648891 | RCV000022096 | deletion |
| GALT | 9 | 34648762 | 34648762 | RCV000022184 | SNV |
| GALT | 9 | 34648763 | 34648763 | RCV000022185 | SNV |
| GALT | 9 | 34648768 | 34648768 | RCV000022186 | SNV |
| GALT | 9 | 34648790 | 34648799 | RCV000022188 | deletion |
| GALT | 9 | 34648816 | 34648816 | RCV000022189 | SNV |
| GALT | 9 | 34648818 | 34648818 | RCV000022190 | SNV |
| GALT | 9 | 34648819 | 34648819 | RCV000022191 | SNV |
| GALT | 9 | 34648823 | 34648823 | RCV000022192 | SNV |
| GALT | 9 | 34648823 | 34648823 | RCV000022193 | SNV |
| GALT | 9 | 34648827 | 34648827 | RCV000022195 | SNV |
| GALT | 9 | 34648839 | 34648841 | RCV000022198 | deletion |
| GALT | 9 | 34648841 | 34648841 | RCV000022199 | SNV |
| GALT | 9 | 34648843 | 34648843 | RCV000078235 | SNV |
| GALT | 9 | 34648848 | 34648848 | RCV000022202 | SNV |
| GALT | 9 | 34648849 | 34648860 | RCV000022203 | deletion |
| GALT | 9 | 34648856 | 34648856 | RCV000022204 | SNV |
| GALT | 9 | 34648861 | 34648863 | RCV000022205; RCV000078236 | indel |
| GALT | 9 | 34648864 | 34648864 | RCV000022206 | SNV |
| GALT | 9 | 34648883 | 34648883 | RCV000022208 | SNV |
| GALT | 9 | 34648885 | 34648885 | RCV000022209 | SNV |
| GALT | 9 | 34648886 | 34648886 | RCV000022210 | SNV |
| GALT | 9 | 34648994 | 34649078 | RCV000022096 | deletion |
| GALT | 9 | 34648994 | 34649078 | RCV000114378 | indel |
| GALT | 9 | 34648998 | 34648998 | RCV000022215 | deletion |
| GALT | 9 | 34649007 | 34649007 | RCV000022216 | SNV |
| GALT | 9 | 34649010 | 34649010 | RCV000022217 | SNV |
| GALT | 9 | 34649018 | 34649018 | RCV000022218; RCV000078238 | SNV |
| GALT | 9 | 34649028 | 34649028 | RCV000022220 | SNV |
| GALT | 9 | 34649029 | 34649029 | RCV000003805; RCV000078239 | SNV |
| GALT | 9 | 34649031 | 34649031 | RCV000022219 | SNV |
| GALT | 9 | 34649039 | 34649039 | RCV000022221 | SNV |
| GALT | 9 | 34649040 | 34649040 | RCV000022222 | SNV |
| GALT | 9 | 34649045 | 34649045 | RCV000022223 | SNV |
| GALT | 9 | 34649046 | 34649046 | RCV000022224 | SNV |
| GALT | 9 | 34649055 | 34649055 | RCV000022226 | SNV |
| GALT | 9 | 34649056 | 34649056 | RCV000022227 | deletion |
| GALT | 9 | 34649057 | 34649057 | RCV000022228 | SNV |
| GALT | 9 | 34649406 | 34649561 | RCV000022096 | deletion |
| GALT | 9 | 34649406 | 34649561 | RCV000114378 | indel |
| GALT | 9 | 34649422 | 34649422 | RCV000022231 | SNV |
| GALT | 9 | 34649424 | 34649424 | RCV000022232 | SNV |
| GALT | 9 | 34649442 | 34649442 | RCV000022233; RCV000003797; | SNV |
| | | | | RCV000078243; RCV000003804; | |
| | | | | RCV000128642 | |
| GALT | 9 | 34649442 | 34649442 | RCV000022233; RCV000003797; | SNV |
| | | | | RCV000078243; RCV000003804; | |
| | | | | RCV000128642 | |
| GALT | 9 | 34649442 | 34649442 | RCV000022233; RCV000003797; | SNV |
| | | | | RCV000078243; RCV000003804; | |
| | | | | RCV000128642 | |
| GALT | 9 | 34649449 | 34649449 | RCV000022235 | SNV |
| GALT | 9 | 34649450 | 34649450 | RCV000022236 | SNV |
| GALT | 9 | 34649451 | 34649451 | RCV000022237 | deletion |
| GALT | 9 | 34649452 | 34649452 | RCV000022238 | SNV |
| GALT | 9 | 34649453 | 34649453 | RCV000022239 | SNV |
| GALT | 9 | 34649454 | 34649454 | RCV000022240 | deletion |
| GALT | 9 | 34649459 | 34649459 | RCV000003801 | SNV |
| GALT | 9 | 34649460 | 34649460 | RCV000022243 | SNV |
| GALT | 9 | 34649461 | 34649461 | RCV000032024 | SNV |
| GALT | 9 | 34649463 | 34649463 | RCV000022244 | SNV |
| GALT | 9 | 34649469 | 34649469 | RCV000022245 | SNV |
| GALT | 9 | 34649469 | 34649469 | RCV000022246 | SNV |
| GALT | 9 | 34649470 | 34649470 | RCV000022247 | SNV |
| GALT | 9 | 34649472 | 34649472 | RCV000022248 | SNV |
| GALT | 9 | 34649476 | 34649476 | RCV000022250 | SNV |
| GALT | 9 | 34649478 | 34649478 | RCV000022251 | deletion |
| GALT | 9 | 34649481 | 34649481 | RCV000022252 | deletion |
| GALT | 9 | 34649482 | 34649482 | RCV000022253 | SNV |
| GALT | 9 | 34649485 | 34649485 | RCV000022254 | SNV |
| GALT | 9 | 34649487 | 34649487 | RCV000078245 | SNV |
| GALT | 9 | 34649488 | 34649488 | RCV000022255 | SNV |
| GALT | 9 | 34649491 | 34649491 | RCV000022256 | SNV |
| GALT | 9 | 34649499 | 34649499 | RCV000003794; RCV000078246 | SNV |
| GALT | 9 | 34649499 | 34649499 | RCV000003807 | SNV |
| GALT | 9 | 34649500 | 34649500 | RCV000022258 | SNV |
| GALT | 9 | 34649500 | 34649500 | RCV000022259 | SNV |
| GALT | 9 | 34649503 | 34649503 | RCV000022260 | SNV |
| GALT | 9 | 34649508 | 34649508 | RCV000022261 | SNV |
| GALT | 9 | 34649520 | 34649520 | RCV000022263 | SNV |
| GALT | 9 | 34649520 | 34649520 | RCV000022264 | SNV |
| GALT | 9 | 34649526 | 34649526 | RCV000022265 | SNV |
| GALT | 9 | 34649532 | 34649532 | RCV000022266; RCV000078213 | SNV |
| GALT | 9 | 34649536 | 34649536 | RCV000022267 | SNV |
| GALT | 9 | 34649549 | 34649549 | RCV000022268 | deletion |
| GALT | 9 | 34649550 | 34649550 | RCV000022269 | SNV |
| GALT | 9 | 34649553 | 34649553 | RCV000022270 | deletion |
| GALT | 9 | 34649559 | 34649559 | RCV000022271 | SNV |
| GALT | 9 | 34650365 | 34650446 | RCV000114378 | indel |
| GALT | 9 | 34650365 | 34650365 | RCV000022275 | SNV |
| GALT | 9 | 34650378 | 34650378 | RCV000022276 | deletion |
| GALT | 9 | 34650384 | 34650389 | RCV000022277 | indel |
| GALT | 9 | 34650404 | 34650404 | RCV000022278 | SNV |
| GALT | 9 | 34650414 | 34650414 | RCV000022279 | SNV |
| GALT | 9 | 34650438 | 34650438 | RCV000022280 | SNV |
| GALT | 9 | 34650444 | 34650444 | RCV000022281 | SNV |
| GALT | 9 | 34650446 | 34650446 | RCV000022282 | SNV |
| GBA | 1 | 155204793 | 155204793 | RCV000004553; RCV000020153 | SNV |
| GBA | 1 | 155204848 | 155204848 | RCV000004552 | SNV |
| GBA | 1 | 155204986 | 155204986 | RCV000020152 | SNV |
| GBA | 1 | 155204987 | 155204987 | RCV000004531; RCV000004529; | SNV |
| | | | | RCV000004530; RCV000004528; | |
| | | | | RCV000020151; RCV000079343 | |
| GBA | 1 | 155204994 | 155204994 | RCV000004534; RCV000004535; | SNV |
| | | | | RCV000004533; RCV000004536; | |
| | | | | RCV000079342 | |
| GBA | 1 | 155204994 | 155204994 | RCV000004534; RCV000004535; | SNV |
| | | | | RCV000004533; RCV000004536; | |
| | | | | RCV000079342 | |
| GBA | 1 | 155205008 | 155205008 | RCV000004534; RCV000004535; | SNV |
| | | | | RCV000004533; RCV000004536; | |
| | | | | RCV000079341 | |
| GBA | 1 | 155205008 | 155205008 | RCV000004534; RCV000004535; | SNV |
| | | | | RCV000004533; RCV000004536; | |
| | | | | RCV000079341 | |
| GBA | 1 | 155205043 | 155205043 | RCV000004512; RCV000004509; | SNV |
| | | | | RCV000004510; RCV000004511; | |
| | | | | RCV000004513; RCV000020150; | |
| | | | | RCV000079339; RCV000004534; | |
| | | | | RCV000004535; RCV000004533; | |
| | | | | RCV000004536 | |
| GBA | 1 | 155205043 | 155205043 | RCV000004512; RCV000004509; | SNV |
| | | | | RCV000004510; RCV000004511; | |
| | | | | RCV000004513; RCV000020150; | |
| | | | | RCV000079339; RCV000004534; | |
| | | | | RCV000004535; RCV000004533; | |
| | | | | RCV000004536 | |
| GBA | 1 | 155205043 | 155205043 | RCV000079340 | SNV |
| GBA | 1 | 155205499 | 155205499 | RCV000004514 | SNV |
| GBA | 1 | 155205517 | 155205517 | RCV000004527; RCV000020149 | SNV |
| GBA | 1 | 155205518 | 155205518 | RCV000004524; RCV000004525; | SNV |
| | | | | RCV000004523; RCV000004526; | |
| | | | | RCV000004522; RCV000055773; | |
| | | | | RCV000079338; RCV000004580; | |
| | | | | RCV000004581 | |
| GBA | 1 | 155205518 | 155205518 | RCV000004524; RCV000004525; | SNV |
| | | | | RCV000004523; RCV000004526; | |
| | | | | RCV000004522; RCV000055773; | |
| | | | | RCV000079338; RCV000004580; | |
| | | | | RCV000004581 | |
| GBA | 1 | 155205541 | 155205541 | RCV000004568 | SNV |
| GBA | 1 | 155205543 | 155205597 | RCV000004554; RCV000004555; | deletion |
| | | | | RCV000020147 | |
| GBA | 1 | 155205551 | 155205551 | RCV000004544 | SNV |
| GBA | 1 | 155205563 | 155205563 | RCV000004520; RCV000004521; | SNV |
| | | | | RCV000020148 | |
| GBA | 1 | 155205614 | 155205614 | RCV000004572; RCV000004571; | SNV |
| | | | | RCV000055772 | |
| GBA | 1 | 155205620 | 155205620 | RCV000079337 | SNV |
| GBA | 1 | 155205632 | 155205632 | RCV000004578 | SNV |
| GBA | 1 | 155205634 | 155205634 | RCV000004516; RCV000004515; | SNV |
| | | | | RCV000004517; RCV000079336 | |
| GBA | 1 | 155205634 | 155205634 | RCV000020146 | SNV |
| GBA | 1 | 155206052 | 155206052 | RCV000004564 | SNV |
| GBA | 1 | 155206068 | 155206068 | RCV000004570; RCV000079334 | SNV |
| GBA | 1 | 155206086 | 155206086 | RCV000004567 | SNV |
| GBA | 1 | 155206089 | 155206089 | RCV000079333 | SNV |
| GBA | 1 | 155206119 | 155206119 | RCV000004563 | SNV |
| GBA | 1 | 155206167 | 155206167 | RCV000004538 | SNV |
| GBA | 1 | 155206170 | 155206170 | RCV000004562; RCV000079331 | SNV |
| GBA | 1 | 155206175 | 155206175 | RCV000041967; RCV000004548 | SNV |
| GBA | 1 | 155206200 | 155206200 | RCV000079330 | SNV |
| GBA | 1 | 155206207 | 155206207 | RCV000004561 | SNV |
| GBA | 1 | 155206211 | 155206211 | RCV000004569 | SNV |
| GBA | 1 | 155206217 | 155206217 | RCV000004560 | SNV |
| GBA | 1 | 155207148 | 155207148 | RCV000004547 | SNV |
| GBA | 1 | 155207244 | 155207244 | RCV000004573; RCV000020159; | SNV |
| | | | | RCV000079357 | |
| GBA | 1 | 155207249 | 155207249 | RCV000004580; RCV000004581 | SNV |
| GBA | 1 | 155207261 | 155207261 | RCV000004577 | SNV |
| GBA | 1 | 155207367 | 155207367 | RCV000004537 | SNV |
| GBA | 1 | 155207368 | 155207368 | RCV000004559 | SNV |
| GBA | 1 | 155207932 | 155207932 | RCV000004541; RCV000004540; | SNV |
| | | | | RCV000004542; RCV000020158; | |
| | | | | RCV000079355 | |
| GBA | 1 | 155207935 | 155207935 | RCV000004551 | SNV |
| GBA | 1 | 155207965 | 155207965 | RCV000079354 | SNV |
| GBA | 1 | 155207983 | 155207983 | RCV000020157 | SNV |
| GBA | 1 | 155208006 | 155208006 | RCV000004558; RCV000004557; | SNV |
| | | | | RCV000020156; RCV000079352 | |
| GBA | 1 | 155208019 | 155208019 | RCV000079351 | SNV |
| GBA | 1 | 155208061 | 155208061 | RCV000079349 | SNV |
| GBA | 1 | 155208310 | 155208310 | RCV000004539 | SNV |
| GBA | 1 | 155208361 | 155208361 | RCV000004538 | SNV |
| GBA | 1 | 155208364 | 155208364 | RCV000004566 | deletion |
| GBA | 1 | 155208387 | 155208387 | RCV000004574; RCV000020155 | SNV |
| GBA | 1 | 155208388 | 155208388 | RCV000079347 | SNV |
| GBA | 1 | 155208409 | 155208409 | RCV000079346 | deletion |
| GBA | 1 | 155208415 | 155208415 | RCV000004550 | SNV |
| GBA | 1 | 155208420 | 155208420 | RCV000004518; RCV000004519; | SNV |
| | | | | RCV000020154 | |
| GBA | 1 | 155208421 | 155208421 | RCV000055774; RCV000079345 | SNV |
| GBA | 1 | 155209507 | 155209507 | RCV000004576; RCV000004575; | SNV |
| | | | | RCV000079344 | |
| GBA | 1 | 155209725 | 155209725 | RCV000004565 | SNV |
| GBA | 1 | 155209730 | 155209730 | RCV000004532 | SNV |
| GBA | 1 | 155209824 | 155209824 | RCV000004556 | SNV |
| GBA | 1 | 155210420 | 155210420 | RCV000079332 | SNV |
| GBA | 1 | 155210442 | 155210443 | RCV000020160 | insertion |
| GBA | 1 | 155210451 | 155210452 | RCV000004543; RCV000079356 | duplication |
| GBA | 1 | 155210464 | 155210464 | RCV000004549 | deletion |
| GBA | 1 | 155210876 | 155210876 | RCV000004546; RCV000004545; | SNV |
| | | | | RCV000032094 | |
| GCDH | 19 | 13006842 | 13006842 | RCV000078254 | SNV |
| GCDH | 19 | 13007018 | 13007018 | RCV000078255 | SNV |
| GCDH | 19 | 13007063 | 13007063 | RCV000002170; RCV000078256 | SNV |
| GCDH | 19 | 13007748 | 13007748 | RCV000002164 | SNV |
| GCDH | 19 | 13007754 | 13007754 | RCV000002162 | SNV |
| GCDH | 19 | 13008527 | 13008527 | RCV000002167 | SNV |
| GCDH | 19 | 13008632 | 13008632 | RCV000002169 | SNV |
| GCDH | 19 | 13008638 | 13008638 | RCV000002166 | SNV |
| GCH1 | 14 | 55310741 | 55310741 | RCV000009869 | SNV |
| GCH1 | 14 | 55310817 | 55310817 | RCV000009865; RCV000009866 | SNV |
| GCH1 | 14 | 55310826 | 55310826 | RCV000009863 | SNV |
| GCH1 | 14 | 55310855 | 55310855 | RCV000009870 | SNV |
| GCH1 | 14 | 55312510 | 55312510 | RCV000009856 | SNV |
| GCH1 | 14 | 55312517 | 55312517 | RCV000009875 | SNV |
| GCH1 | 14 | 55312526 | 55312526 | RCV000009867 | SNV |
| GCH1 | 14 | 55312561 | 55312561 | RCV000009873 | SNV |
| GCH1 | 14 | 55332067 | 55332067 | RCV000009860 | SNV |
| GCH1 | 14 | 55332094 | 55332094 | RCV000009868 | SNV |
| GCH1 | 14 | 55332097 | 55332097 | RCV000009854 | SNV |
| GCH1 | 14 | 55369059 | 55369059 | RCV000009864 | SNV |
| GCH1 | 14 | 55369120 | 55369120 | RCV000009853 | SNV |
| GCH1 | 14 | 55369240 | 55369240 | RCV000009871 | SNV |
| GCH1 | 14 | 55369379 | 55369379 | RCV000009859 | SNV |
| GJB2 | 13 | 20763104 | 20763104 | RCV000037868 | SNV |
| GJB2 | 13 | 20763116 | 20763116 | RCV000018544 | SNV |
| GJB2 | 13 | 20763121 | 20763129 | RCV000037866 | indel |
| GJB2 | 13 | 20763170 | 20763170 | RCV000018531; RCV000037863 | SNV |
| GJB2 | 13 | 20763170 | 20763170 | RCV000022511 | SNV |
| GJB2 | 13 | 20763186 | 20763186 | RCV000018553 | SNV |
| GJB2 | 13 | 20763234 | 20763234 | RCV000020573; RCV000037860 | SNV |
| GJB2 | 13 | 20763245 | 20763245 | RCV000018551 | SNV |
| GJB2 | 13 | 20763265 | 20763265 | RCV000037856 | SNV |
| GJB2 | 13 | 20763293 | 20763293 | RCV000018543; RCV000018542 | SNV |
| GJB2 | 13 | 20763294 | 20763294 | RCV000018533; RCV000037853 | SNV |
| GJB2 | 13 | 20763295 | 20763295 | RCV000037852 | SNV |
| GJB2 | 13 | 20763305 | 20763305 | RCV000037851 | SNV |
| GJB2 | 13 | 20763351 | 20763351 | RCV000037846 | SNV |
| GJB2 | 13 | 20763356 | 20763356 | RCV000037844; RCV000080375 | SNV |
| GJB2 | 13 | 20763361 | 20763363 | RCV000018530; RCV000037841 | deletion |
| GJB2 | 13 | 20763382 | 20763382 | RCV000020568 | SNV |
| GJB2 | 13 | 20763395 | 20763408 | RCV000037836; RCV000080370 | deletion |
| GJB2 | 13 | 20763421 | 20763422 | RCV000037835 | deletion |
| GJB2 | 13 | 20763438 | 20763438 | RCV000037834 | SNV |
| GJB2 | 13 | 20763452 | 20763452 | RCV000018541; RCV000037831; | SNV |
| | | | | RCV000080369 | |
| GJB2 | 13 | 20763471 | 20763471 | RCV000018560; RCV000037830 | SNV |
| GJB2 | 13 | 20763471 | 20763471 | RCV000018564; RCV000037829 | SNV |
| GJB2 | 13 | 20763486 | 20763486 | RCV000018539; RCV000018538; | deletion |
| | | | | RCV000037827 | |
| GJB2 | 13 | 20763490 | 20763490 | RCV000018524; RCV000037826 | SNV |
| GJB2 | 13 | 20763492 | 20763492 | RCV000018526; RCV000037825; | SNV |
| | | | | RCV000080368 | |
| GJB2 | 13 | 20763497 | 20763497 | RCV000018554; RCV000018555 | SNV |
| GJB2 | 13 | 20763498 | 20763498 | RCV000018535; RCV000037823 | SNV |
| GJB2 | 13 | 20763503 | 20763503 | RCV000018565 | SNV |
| GJB2 | 13 | 20763525 | 20763525 | RCV000018536 | SNV |
| GJB2 | 13 | 20763527 | 20763527 | RCV000037819 | SNV |
| GJB2 | 13 | 20763545 | 20763545 | RCV000018540 | SNV |
| GJB2 | 13 | 20763546 | 20763546 | RCV000018562 | SNV |
| GJB2 | 13 | 20763552 | 20763552 | RCV000037815 | SNV |
| GJB2 | 13 | 20763554 | 20763554 | RCV000018534; RCV000037817 | deletion |
| GJB2 | 13 | 20763559 | 20763559 | RCV000018558 | SNV |
| GJB2 | 13 | 20763573 | 20763573 | RCV000018546; RCV000018547 | SNV |
| GJB2 | 13 | 20763573 | 20763573 | RCV000018556 | SNV |
| GJB2 | 13 | 20763582 | 20763582 | RCV000018529; RCV000037816; | SNV |
| | | | | RCV000080366 | |
| GJB2 | 13 | 20763587 | 20763587 | RCV000022510; RCV000018561 | SNV |
| GJB2 | 13 | 20763589 | 20763589 | RCV000018545 | SNV |
| GJB2 | 13 | 20763590 | 20763590 | RCV000018559 | SNV |
| GJB2 | 13 | 20763602 | 20763602 | RCV000037813 | SNV |
| GJB2 | 13 | 20763612 | 20763612 | RCV000018550; RCV000037812; | SNV |
| | | | | RCV000080365 | |
| GJB2 | 13 | 20763620 | 20763620 | RCV000018523; RCV000037810; | SNV |
| | | | | RCV000080364 | |
| GJB2 | 13 | 20763620 | 20763620 | RCV000037811 | SNV |
| GJB2 | 13 | 20763626 | 20763626 | RCV000037873 | SNV |
| GJB2 | 13 | 20763650 | 20763650 | RCV000018525; RCV000037871 | SNV |
| GJB2 | 13 | 20763653 | 20763690 | RCV000037837 | deletion |
| GJB2 | 13 | 20763665 | 20763665 | RCV000020575 | SNV |
| GJB2 | 13 | 20763671 | 20763671 | RCV000018549 | SNV |
| GJB2 | 13 | 20763677 | 20763677 | RCV000037855 | SNV |
| GJB2 | 13 | 20763686 | 20763686 | RCV000018528; RCV000018527; | deletion |
| | | | | RCV000037843; RCV000080373 | |
| GJB2 | 13 | 20763686 | 20763686 | RCV000020570; RCV000037842; | SNV |
| | | | | RCV000080372 | |
| GJB2 | 13 | 20763686 | 20763686 | RCV000080374 | duplication |
| GJB2 | 13 | 20763687 | 20763687 | RCV000018548 | SNV |
| GJB2 | 13 | 20763687 | 20763687 | RCV000037839; RCV000080371 | SNV |
| GJB2 | 13 | 20763702 | 20763702 | RCV000037820 | SNV |
| GJB2 | 13 | 20763712 | 20763712 | RCV000037874 | SNV |
| GJB2 | 13 | 20763720 | 20763720 | RCV000037821 | SNV |
| GJB3 | 1 | 35250397 | 35250397 | RCV000006855 | SNV |
| GJB3 | 1 | 35250398 | 35250398 | RCV000006856 | SNV |
| GJB3 | 1 | 35250464 | 35250464 | RCV000006864 | SNV |
| GJB3 | 1 | 35250488 | 35250488 | RCV000006862 | SNV |
| GJB3 | 1 | 35250619 | 35250619 | RCV000006857 | SNV |
| GJB3 | 1 | 35250784 | 35250784 | RCV000006861 | SNV |
| GJB3 | 1 | 35250860 | 35250860 | RCV000006865 | SNV |
| GJB3 | 1 | 35250901 | 35250901 | RCV000006859 | SNV |
| GJB3 | 1 | 35250910 | 35250910 | RCV000006858 | SNV |
| GJB3 | 1 | 35250943 | 35250943 | RCV000006866 | SNV |
| GJB6 | 13 | 20796931 | 20796931 | RCV000081460 | duplication |
| GJB6 | 13 | 20797357 | 20797357 | RCV000005883 | SNV |
| GJB6 | 13 | 20797359 | 20797359 | RCV000081458 | duplication |
| GJB6 | 13 | 20797510 | 20797510 | RCV000005886 | SNV |
| GJB6 | 13 | 20797559 | 20797560 | RCV000038711 | indel |
| GJB6 | 13 | 20797589 | 20797589 | RCV000005882 | SNV |
| GJB6 | 13 | 20797606 | 20797606 | RCV000088666 | SNV |
| GNMT | 6 | 42928654 | 42928654 | RCV000004386 | SNV |
| GNMT | 6 | 42930887 | 42930887 | RCV000004387 | SNV |
| HADH | 4 | 108911206 | 108911206 | RCV000008482 | SNV |
| HADH | 4 | 108930953 | 108930953 | RCV000008483 | SNV |
| HADH | 4 | 108948913 | 108948913 | RCV000032678 | SNV |
| HADH | 4 | 108954395 | 108954395 | RCV000008484 | SNV |
| HADHA | 2 | 26417450 | 26417450 | RCV000009271 | SNV |
| HADHA | 2 | 26418053 | 26418053 | RCV000009266; RCV000009267 | SNV |
| HADHA | 2 | 26427019 | 26427019 | RCV000009268 | SNV |
| HADHA | 2 | 26432709 | 26432709 | RCV000009273 | SNV |
| HADHA | 2 | 26437316 | 26437316 | RCV000009275 | SNV |
| HADHA | 2 | 26437359 | 26437359 | RCV000009276 | SNV |
| HADHA | 2 | 26437385 | 26437385 | RCV000009274 | SNV |
| HADHA | 2 | 26461825 | 26461825 | RCV000078334 | SNV |
| HADHB | 2 | 26486320 | 26486320 | RCV000015970 | SNV |
| HADHB | 2 | 26502112 | 26502112 | RCV000015971 | SNV |
| HADHB | 2 | 26502160 | 26502160 | RCV000015969 | SNV |
| HADHB | 2 | 26508381 | 26508381 | RCV000015972 | SNV |
| HADHB | 2 | 26508414 | 26508414 | RCV000015974 | SNV |
| HBA2 | 16 | 222912 | 222912 | RCV000016985 | deletion |
| HBA2 | 16 | 222913 | 222913 | RCV000016929 | SNV |
| HBA2 | 16 | 222980 | 222980 | RCV000016977 | SNV |
| HBA2 | 16 | 222981 | 222981 | RCV000016976 | SNV |
| HBA2 | 16 | 223206 | 223206 | RCV000016979; RCV000016978 | SNV |
| HBA2 | 16 | 223214 | 223214 | RCV000016900; RCV000016901 | SNV |
| HBA2 | 16 | 223519 | 223519 | RCV000016930 | SNV |
| HBB | 11 | 5246884 | 5246887 | RCV000016876 | indel |
| HBB | 11 | 5246887 | 5246889 | RCV000016665 | deletion |
| HBB | 11 | 5246889 | 5246889 | RCV000016664; RCV000022611 | SNV |
| HBB | 11 | 5246908 | 5246908 | RCV000016658 | SNV |
| HBB | 11 | 5246928 | 5246928 | RCV000016775; RCV000016776; | SNV |
| | | | | RCV000016777 | |
| HBB | 11 | 5246928 | 5246929 | RCV000016681; RCV000016682 | indel |
| HBB | 11 | 5246935 | 5246935 | RCV000016393; RCV000016392 | SNV |
| HBB | 11 | 5246940 | 5246940 | RCV000016599; RCV000016598; | SNV |
| | | | | RCV000016600; RCV000029991 | |
| HBB | 11 | 5246944 | 5246944 | RCV000016687 | deletion |
| HBB | 11 | 5246950 | 5246951 | RCV000016668 | duplication |
| HBB | 11 | 5246952 | 5246952 | RCV000016747; RCV000016746 | SNV |
| HBB | 11 | 5247806 | 5247806 | RCV000016696; RCV000020332 | SNV |
| HBB | 11 | 5247807 | 5247807 | RCV000016789 | SNV |
| HBB | 11 | 5247827 | 5247827 | RCV000016446; RCV000016443; | SNV |
| | | | | RCV000016444; RCV000016445; | |
| | | | | RCV000016807 | |
| HBB | 11 | 5247827 | 5247827 | RCV000016446; RCV000016443; | SNV |
| | | | | RCV000016444; RCV000016445; | |
| | | | | RCV000016807 | |
| HBB | 11 | 5247835 | 5247835 | RCV000029975 | duplication |
| HBB | 11 | 5247838 | 5247839 | RCV000016685 | insertion |
| HBB | 11 | 5247851 | 5247851 | RCV000016766 | SNV |
| HBB | 11 | 5247871 | 5247871 | RCV000029971 | deletion |
| HBB | 11 | 5247875 | 5247875 | RCV000016827 | SNV |
| HBB | 11 | 5247892 | 5247892 | RCV000016676 | deletion |
| HBB | 11 | 5247901 | 5247905 | RCV000016826 | indel |
| HBB | 11 | 5247905 | 5247906 | RCV000016675 | duplication |
| HBB | 11 | 5247905 | 5247906 | RCV000016690 | duplication |
| HBB | 11 | 5247914 | 5247914 | RCV000016301; RCV000029969 | SNV |
| HBB | 11 | 5247918 | 5247919 | RCV000029968 | deletion |
| HBB | 11 | 5247921 | 5247921 | RCV000029967 | deletion |
| HBB | 11 | 5247929 | 5247929 | RCV000016686 | deletion |
| HBB | 11 | 5247938 | 5247938 | RCV000016661 | SNV |
| HBB | 11 | 5247940 | 5247940 | RCV000016666; RCV000016667 | SNV |
| HBB | 11 | 5247978 | 5247979 | RCV000016744 | duplication |
| HBB | 11 | 5247987 | 5247987 | RCV000016671; RCV000020329 | deletion |
| HBB | 11 | 5247992 | 5247992 | RCV000016660 | SNV |
| HBB | 11 | 5247993 | 5247995 | RCV000016277; RCV000016278 | deletion |
| HBB | 11 | 5247993 | 5247996 | RCV000016673; RCV000020328 | deletion |
| HBB | 11 | 5247994 | 5247994 | RCV000016373; RCV000016371; | SNV |
| | | | | RCV000016372 | |
| HBB | 11 | 5248002 | 5248008 | RCV000016684 | deletion |
| HBB | 11 | 5248004 | 5248004 | RCV000016656; RCV000020327 | SNV |
| HBB | 11 | 5248005 | 5248006 | RCV000016817 | deletion |
| HBB | 11 | 5248008 | 5248008 | RCV000016659 | SNV |
| HBB | 11 | 5248010 | 5248010 | RCV000016688 | deletion |
| HBB | 11 | 5248014 | 5248014 | RCV000016662 | SNV |
| HBB | 11 | 5248014 | 5248014 | RCV000016680 | deletion |
| HBB | 11 | 5248027 | 5248029 | RCV000016700; RCV000030009 | deletion |
| HBB | 11 | 5248028 | 5248029 | RCV000016821 | insertion |
| HBB | 11 | 5248029 | 5248029 | RCV000016620; RCV000016619 | SNV |
| HBB | 11 | 5248159 | 5248176 | RCV000016703 | deletion |
| HBB | 11 | 5248159 | 5248159 | RCV000016694; RCV000020340 | SNV |
| HBB | 11 | 5248159 | 5248159 | RCV000016695 | SNV |
| HBB | 11 | 5248160 | 5248160 | RCV000016432; RCV000030007 | SNV |
| HBB | 11 | 5248160 | 5248160 | RCV000030006 | SNV |
| HBB | 11 | 5248166 | 5248166 | RCV000016612; RCV000016611 | SNV |
| HBB | 11 | 5248167 | 5248168 | RCV000016689 | duplication |
| HBB | 11 | 5248170 | 5248170 | RCV000016440; RCV000016439; | SNV |
| | | | | RCV000016441 | |
| HBB | 11 | 5248172 | 5248172 | RCV000016820 | SNV |
| HBB | 11 | 5248172 | 5248173 | RCV000016829 | insertion |
| HBB | 11 | 5248173 | 5248173 | RCV000016332; RCV000016330; | SNV |
| | | | | RCV000016329; RCV000016331; | |
| | | | | RCV000016617 | |
| HBB | 11 | 5248173 | 5248173 | RCV000016332; RCV000016330; | SNV |
| | | | | RCV000016329; RCV000016331; | |
| | | | | RCV000016617 | |
| HBB | 11 | 5248177 | 5248177 | RCV000016717; RCV000030002; | SNV |
| | | | | RCV000020339 | |
| HBB | 11 | 5248193 | 5248193 | RCV000016480; RCV000016479; | SNV |
| | | | | RCV000016481; RCV000020338 | |
| HBB | 11 | 5248200 | 5248200 | RCV000016655; RCV000020337 | SNV |
| HBB | 11 | 5248201 | 5248201 | RCV000016670 | deletion |
| HBB | 11 | 5248205 | 5248205 | RCV000016657 | SNV |
| HBB | 11 | 5248206 | 5248207 | RCV000016683 | duplication |
| HBB | 11 | 5248216 | 5248216 | RCV000016679 | deletion |
| HBB | 11 | 5248224 | 5248225 | RCV000016672; RCV000029974; | duplication |
| | | | | RCV000020331 | |
| HBB | 11 | 5248226 | 5248227 | RCV000016669; RCV000029972; | deletion |
| | | | | RCV000020330 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016286; RCV000030905; | SNV |
| | | | | RCV000016575; RCV000016573; | |
| | | | | RCV000016574; RCV000016576; | |
| | | | | RCV000016577; RCV000016579; | |
| | | | | RCV000016580; RCV000016877; | |
| | | | | RCV000016879 | |
| HBB | 11 | 5248232 | 5248232 | RCV000016674 | deletion |
| HBB | 11 | 5248232 | 5248233 | RCV000029966 | indel |
| HBB | 11 | 5248233 | 5248233 | RCV000016251; RCV000016285; | SNV |
| | | | | RCV000016284 | |
| HBB | 11 | 5248233 | 5248233 | RCV000016251; RCV000016285; | SNV |
| | | | | RCV000016284 | |
| HBB | 11 | 5248234 | 5248235 | RCV000016678 | deletion |
| HBB | 11 | 5248248 | 5248248 | RCV000016770 | deletion |
| HBB | 11 | 5248248 | 5248248 | RCV000016824 | SNV |
| HBB | 11 | 5248249 | 5248249 | RCV000016783 | SNV |
| HBB | 11 | 5248250 | 5248250 | RCV000016691 | SNV |
| HBB | 11 | 5248250 | 5248250 | RCV000016692; RCV000016693; | SNV |
| | | | | RCV000029976 | |
| HLCS | 21 | 38132082 | 38132082 | RCV000001987 | SNV |
| HLCS | 21 | 38132112 | 38132112 | RCV000001985 | SNV |
| HLCS | 21 | 38137345 | 38137345 | RCV000001988 | SNV |
| HLCS | 21 | 38137471 | 38137471 | RCV000001986 | SNV |
| HLCS | 21 | 38309035 | 38309035 | RCV000001984 | SNV |
| HLCS | 21 | 38309098 | 38309098 | RCV000001991 | SNV |
| HMGCL | 1 | 24130931 | 24130931 | RCV000012736 | SNV |
| HMGCL | 1 | 24144010 | 24144010 | RCV000012733 | SNV |
| HMGCL | 1 | 24147022 | 24147022 | RCV000012735; RCV000078342 | SNV |
| HPD | 12 | 122277904 | 122277904 | RCV000001642 | SNV |
| HPD | 12 | 122284825 | 122284825 | RCV000001640 | SNV |
| HPD | 12 | 122285117 | 122285117 | RCV000001641 | SNV |
| HPD | 12 | 122287632 | 122287632 | RCV000001639 | SNV |
| HPD | 12 | 122295335 | 122295335 | RCV000001643 | SNV |
| HSD17B10 | X | 53458393 | 53458393 | RCV000012199 | SNV |
| HSD17B10 | X | 53458398 | 53458398 | RCV000012197 | SNV |
| HSD17B10 | X | 53458767 | 53458767 | RCV000012198 | SNV |
| HSD17B10 | X | 53459034 | 53459034 | RCV000012195 | SNV |
| HSD17B10 | X | 53459058 | 53459058 | RCV000012196 | SNV |
| IDUA | 4 | 981630 | 981630 | RCV000012689 | SNV |
| IDUA | 4 | 981646 | 981646 | RCV000012684; RCV000078386 | SNV |
| IDUA | 4 | 981704 | 981704 | RCV000012697 | SNV |
| IDUA | 4 | 995263 | 995263 | RCV000078394 | SNV |
| IDUA | 4 | 995875 | 995875 | RCV000012698 | SNV |
| IDUA | 4 | 995905 | 995905 | RCV000012690 | SNV |
| IDUA | 4 | 996121 | 996121 | RCV000012703; RCV000012702 | SNV |
| IDUA | 4 | 996129 | 996129 | RCV000078370 | SNV |
| IDUA | 4 | 996175 | 996175 | RCV000012700 | SNV |
| IDUA | 4 | 996180 | 996180 | RCV000012691 | SNV |
| IDUA | 4 | 996535 | 996535 | RCV000012683; RCV000078374 | SNV |
| IDUA | 4 | 996555 | 996555 | RCV000012686; RCV000078375 | SNV |
| IDUA | 4 | 996555 | 996555 | RCV000012686; RCV000078375 | SNV |
| IDUA | 4 | 996890 | 996890 | RCV000012694; RCV000078381 | SNV |
| IDUA | 4 | 996896 | 996896 | RCV000012693 | SNV |
| IDUA | 4 | 997206 | 997206 | RCV000012685 | SNV |
| IDUA | 4 | 997871 | 997871 | RCV000078385 | deletion |
| IDUA | 4 | 998074 | 998074 | RCV000012699 | SNV |
| IDUA | 4 | 998080 | 998080 | RCV000012692 | SNV |
| IDUA | 4 | 998093 | 998093 | RCV000087088 | SNV |
| IDUA | 4 | 998179 | 998179 | RCV000012695 | SNV |
| IDUA | 4 | 998181 | 998181 | RCV000012686 | SNV |
| IL2RG | X | 70328128 | 70328128 | RCV000010701 | SNV |
| IL2RG | X | 70328173 | 70328173 | RCV000010706 | SNV |
| IL2RG | X | 70328186 | 70328186 | RCV000030058 | SNV |
| IL2RG | X | 70328449 | 70328449 | RCV000010709 | SNV |
| IL2RG | X | 70329125 | 70329125 | RCV000030057 | SNV |
| IL2RG | X | 70329171 | 70329171 | RCV000010710 | SNV |
| IL2RG | X | 70329173 | 70329173 | RCV000030056 | SNV |
| IL2RG | X | 70330142 | 70330142 | RCV000010705 | SNV |
| IL2RG | X | 70330145 | 70330145 | RCV000030055 | SNV |
| IL2RG | X | 70330356 | 70330356 | RCV000010711 | SNV |
| IL2RG | X | 70330453 | 70330453 | RCV000010699 | SNV |
| IL2RG | X | 70330465 | 70330465 | RCV000010708 | SNV |
| IL2RG | X | 70330467 | 70330467 | RCV000010703 | SNV |
| IL2RG | X | 70330494 | 70330494 | RCV000030054 | SNV |
| IL2RG | X | 70330830 | 70330830 | RCV000010702 | SNV |
| IVD | 15 | 40698153 | 40698153 | RCV000003743 | SNV |
| IVD | 15 | 40699840 | 40699840 | RCV000003748 | SNV |
| IVD | 15 | 40702937 | 40702938 | RCV000079998 | deletion |
| IVD | 15 | 40703500 | 40703500 | RCV000080000 | deletion |
| IVD | 15 | 40703799 | 40703799 | RCV000003744 | SNV |
| IVD | 15 | 40707653 | 40707653 | RCV000003749; RCV000080003 | SNV |
| IVD | 15 | 40708555 | 40708555 | RCV000079995 | SNV |
| MAT1A | 10 | 82034291 | 82034291 | RCV000001263 | SNV |
| MAT1A | 10 | 82034355 | 82034355 | RCV000001270 | SNV |
| MAT1A | 10 | 82034395 | 82034395 | RCV000001261 | SNV |
| MAT1A | 10 | 82034810 | 82034810 | RCV000001264 | SNV |
| MAT1A | 10 | 82034933 | 82034933 | RCV000001267 | SNV |
| MAT1A | 10 | 82034934 | 82034934 | RCV000001269 | SNV |
| MAT1A | 10 | 82045273 | 82045273 | RCV000001262 | SNV |
| MCCC1 | 3 | 182733325 | 182733325 | RCV000002011 | deletion |
| MCCC1 | 3 | 182751856 | 182751856 | RCV000002010 | SNV |
| MCCC1 | 3 | 182755006 | 182755006 | RCV000002008 | SNV |
| MCCC1 | 3 | 182755220 | 182755220 | RCV000002012 | SNV |
| MCCC1 | 3 | 182756881 | 182756881 | RCV000002009 | SNV |
| MCCC1 | 3 | 182756914 | 182756914 | RCV000081990 | SNV |
| MCCC1 | 3 | 182759467 | 182759467 | RCV000002007 | SNV |
| MCCC1 | 3 | 182763210 | 182763210 | RCV000081989 | deletion |
| MCCC1 | 3 | 182763310 | 182763310 | RCV000002006; RCV000081995 | SNV |
| MCCC2 | 5 | 70895499 | 70895499 | RCV000001997; RCV000082095 | SNV |
| MCCC2 | 5 | 70895584 | 70895584 | RCV000082096 | SNV |
| MCCC2 | 5 | 70898413 | 70898413 | RCV000001998 | SNV |
| MCCC2 | 5 | 70898448 | 70898448 | RCV000002000 | SNV |
| MCCC2 | 5 | 70900188 | 70900188 | RCV000001996 | duplication |
| MCCC2 | 5 | 70900240 | 70900240 | RCV000002003 | SNV |
| MCCC2 | 5 | 70928012 | 70928012 | RCV000002001 | SNV |
| MCCC2 | 5 | 70930804 | 70930804 | RCV000002004 | SNV |
| MCCC2 | 5 | 70931003 | 70931003 | RCV000001999 | SNV |
| MCCC2 | 5 | 70945016 | 70945016 | RCV000002002 | SNV |
| MCEE | 2 | 71351575 | 71351575 | RCV000002434 | SNV |
| MLYCD | 16 | 83932757 | 83932757 | RCV000004274 | SNV |
| MLYCD | 16 | 83932868 | 83932868 | RCV000004275 | SNV |
| MLYCD | 16 | 83940623 | 83940623 | RCV000004270 | SNV |
| MLYCD | 16 | 83941768 | 83941768 | RCV000081531 | indel |
| MMAA | 4 | 146560574 | 146560574 | RCV000003308 | SNV |
| MMAA | 4 | 146560724 | 146560724 | RCV000003310 | SNV |
| MMAA | 4 | 146567195 | 146567195 | RCV000003309 | SNV |
| MMAA | 4 | 146576363 | 146576363 | RCV000082687 | deletion |
| MMAB | 12 | 109998861 | 109998861 | RCV000082327 | SNV |
| MMAB | 12 | 109998873 | 109998873 | RCV000003241 | SNV |
| MMADHC | 2 | 150426603 | 150426603 | RCV000000797 | SNV |
| MMADHC | 2 | 150426631 | 150426631 | RCV000000803 | SNV |
| MMADHC | 2 | 150426633 | 150426633 | RCV000000799 | SNV |
| MMADHC | 2 | 150432289 | 150432289 | RCV000000798 | SNV |
| MMADHC | 2 | 150433009 | 150433010 | RCV000000804 | duplication |
| MMADHC | 2 | 150435992 | 150435993 | RCV000000802 | duplication |
| MMADHC | 2 | 150436157 | 150436157 | RCV000000801 | SNV |
| MMADHC | 2 | 150438731 | 150438738 | RCV000000800 | deletion |
| MTHFR | 1 | 11851273 | 11851273 | RCV000003705 | SNV |
| MTHFR | 1 | 11854823 | 11854823 | RCV000003706 | SNV |
| MTHFR | 1 | 11855171 | 11855171 | RCV000003701 | SNV |
| MTHFR | 1 | 11855215 | 11855215 | RCV000003700 | SNV |
| MTHFR | 1 | 11855218 | 11855218 | RCV000003707 | SNV |
| MTHFR | 1 | 11856378 | 11856378 | RCV000003697 | SNV |
| MTHFR | 1 | 11860308 | 11860308 | RCV000003695 | SNV |
| MTHFR | 1 | 11861223 | 11861223 | RCV000003696 | SNV |
| MTR | 1 | 236998886 | 236998886 | RCV000015358 | SNV |
| MTR | 1 | 237015878 | 237015878 | RCV000015356 | SNV |
| MTR | 1 | 237048502 | 237048502 | RCV000015350 | SNV |
| MTR | 1 | 237058770 | 237058770 | RCV000015348 | SNV |
| MTR | 1 | 237060320 | 237060320 | RCV000015357 | SNV |
| MTRR | 5 | 7891518 | 7891518 | RCV000007449 | SNV |
| MTRR | 5 | 7892928 | 7892928 | RCV000007446 | SNV |
| MUT | 6 | 49399544 | 49399544 | RCV000001958; RCV000078445 | SNV |
| MUT | 6 | 49403186 | 49403186 | RCV000001962 | SNV |
| MUT | 6 | 49408008 | 49408008 | RCV000001961 | SNV |
| MUT | 6 | 49419381 | 49419381 | RCV000001956 | SNV |
| MUT | 6 | 49425502 | 49425502 | RCV000001963 | SNV |
| MUT | 6 | 49425514 | 49425514 | RCV000001965 | SNV |
| MUT | 6 | 49426831 | 49426831 | RCV000001959 | SNV |
| MUT | 6 | 49426858 | 49426858 | RCV000001964 | SNV |
| MUT | 6 | 49426867 | 49426867 | RCV000001955 | SNV |
| MUT | 6 | 49426902 | 49426902 | RCV000001957 | SNV |
| MUT | 6 | 49427089 | 49427089 | RCV000078448 | SNV |
| MUT | 6 | 49427128 | 49427128 | RCV000001954 | SNV |
| NGLY1 | 3 | 25761025 | 25761025 | RCV000043662 | deletion |
| NGLY1 | 3 | 25761670 | 25761670 | RCV000114364 | SNV |
| NGLY1 | 3 | 25773865 | 25773865 | RCV000114362 | duplication |
| NGLY1 | 3 | 25775416 | 25775418 | RCV000114363 | deletion |
| NGLY1 | 3 | 25775422 | 25775422 | RCV000043663 | SNV |
| NPC1 | 18 | 21113434 | 21113434 | RCV000003099 | SNV |
| NPC1 | 18 | 21115443 | 21115443 | RCV000003093 | SNV |
| NPC1 | 18 | 21115647 | 21115647 | RCV000003098 | SNV |
| NPC1 | 18 | 21116700 | 21116700 | RCV000003101 | SNV |
| NPC1 | 18 | 21116722 | 21116722 | RCV000020231 | SNV |
| NPC1 | 18 | 21116775 | 21116775 | RCV000003092 | SNV |
| NPC1 | 18 | 21116778 | 21116778 | RCV000003106; RCV000078477 | SNV |
| NPC1 | 18 | 21118528 | 21118528 | RCV000003100 | SNV |
| NPC1 | 18 | 21118573 | 21118573 | RCV000003103 | SNV |
| NPC1 | 18 | 21118573 | 21118573 | RCV000020229 | SNV |
| NPC1 | 18 | 21118573 | 21118573 | RCV000020230; RCV000003094 | SNV |
| NPC1 | 18 | 21118615 | 21118615 | RCV000003110 | SNV |
| NPC1 | 18 | 21119357 | 21119357 | RCV000003102 | SNV |
| NPC1 | 18 | 21119382 | 21119382 | RCV000003105 | SNV |
| NPC1 | 18 | 21119775 | 21119775 | RCV000119336 | duplication |
| NPC1 | 18 | 21119787 | 21119787 | RCV000003091 | SNV |
| NPC1 | 18 | 21119905 | 21119905 | RCV000003096 | SNV |
| NPC1 | 18 | 21121277 | 21121277 | RCV000119335 | SNV |
| NPC1 | 18 | 21121319 | 21121319 | RCV000020226 | SNV |
| NPC1 | 18 | 21121341 | 21121341 | RCV000119334 | duplication |
| NPC1 | 18 | 21123433 | 21123434 | RCV000119333 | deletion |
| NPC1 | 18 | 21123468 | 21123468 | RCV000078472 | duplication |
| NPC1 | 18 | 21123487 | 21123487 | RCV000119332 | SNV |
| NPC1 | 18 | 21124310 | 21124310 | RCV000119331 | SNV |
| NPC1 | 18 | 21124384 | 21124384 | RCV000119330 | SNV |
| NPC1 | 18 | 21125039 | 21125039 | RCV000119329 | SNV |
| NPC1 | 18 | 21125071 | 21125071 | RCV000119328 | deletion |
| NPC1 | 18 | 21134722 | 21134722 | RCV000119327 | SNV |
| NPC1 | 18 | 21134773 | 21134773 | RCV000119326 | SNV |
| NPC1 | 18 | 21136400 | 21136400 | RCV000003104 | SNV |
| NPC1 | 18 | 21136503 | 21136503 | RCV000119325 | deletion |
| NPC1 | 18 | 21141425 | 21141425 | RCV000003108 | SNV |
| NPC1 | 18 | 21148834 | 21148834 | RCV000119338 | duplication |
| NPC1 | 18 | 21148913 | 21148913 | RCV000003112 | SNV |
| NPC2 | 14 | 74947404 | 74947404 | RCV000087100 | SNV |
| NPC2 | 14 | 74947410 | 74947410 | RCV000009006 | SNV |
| NPC2 | 14 | 74951123 | 74951123 | RCV000009007 | SNV |
| NPC2 | 14 | 74951129 | 74951129 | RCV000009001 | SNV |
| NPC2 | 14 | 74951149 | 74951149 | RCV000020647 | deletion |
| NPC2 | 14 | 74951186 | 74951186 | RCV000020646 | SNV |
| NPC2 | 14 | 74951282 | 74951282 | RCV000009003 | SNV |
| NPC2 | 14 | 74953081 | 74953081 | RCV000020644 | SNV |
| NPC2 | 14 | 74953089 | 74953089 | RCV000020643 | SNV |
| NPC2 | 14 | 74953107 | 74953107 | RCV000009004 | SNV |
| NPC2 | 14 | 74959920 | 74959920 | RCV000008998 | SNV |
| NPC2 | 14 | 74959951 | 74959951 | RCV000020645 | deletion |
| NPC2 | 14 | 74959975 | 74959975 | RCV000119339 | SNV |
| OPA3 | 19 | 46056897 | 46056897 | RCV000020909 | SNV |
| OPA3 | 19 | 46056975 | 46056992 | RCV000004464 | deletion |
| OPA3 | 19 | 46056999 | 46056999 | RCV000004463 | SNV |
| OPA3 | 19 | 46057035 | 46057035 | RCV000004462 | SNV |
| PAH | 12 | 103234177 | 103234177 | RCV000078510 | SNV |
| PAH | 12 | 103234250 | 103234250 | RCV000000648; RCV000088815 | SNV |
| PAH | 12 | 103234252 | 103234252 | RCV000000624; RCV000078508 | SNV |
| PAH | 12 | 103234253 | 103234253 | RCV000106346 | SNV |
| PAH | 12 | 103234255 | 103234255 | RCV000000623; RCV000088813 | SNV |
| PAH | 12 | 103234270 | 103234270 | RCV000000643; RCV000088806 | SNV |
| PAH | 12 | 103234271 | 103234271 | RCV000000607; RCV000078507 | SNV |
| PAH | 12 | 103234273 | 103234273 | RCV000000667; RCV000088804 | SNV |
| PAH | 12 | 103234285 | 103234285 | RCV000078506 | SNV |
| PAH | 12 | 103237427 | 103237427 | RCV000106344 | SNV |
| PAH | 12 | 103237443 | 103237443 | RCV000106343 | SNV |
| PAH | 12 | 103237452 | 103237452 | RCV000106342 | SNV |
| PAH | 12 | 103237454 | 103237454 | RCV000000657; RCV000000656; | SNV |
| | | | | RCV000078503 | |
| PAH | 12 | 103237460 | 103237460 | RCV000106341 | SNV |
| PAH | 12 | 103237461 | 103237461 | RCV000000650; RCV000088774 | SNV |
| PAH | 12 | 103237484 | 103237484 | RCV000000660; RCV000078502 | SNV |
| PAH | 12 | 103237547 | 103237547 | RCV000000658; RCV000088747 | SNV |
| PAH | 12 | 103237555 | 103237555 | RCV000000626; RCV000088745 | SNV |
| PAH | 12 | 103237555 | 103237555 | RCV000078501 | SNV |
| PAH | 12 | 103238134 | 103238134 | RCV000000646; RCV000078499 | SNV |
| PAH | 12 | 103238137 | 103238137 | RCV000078498 | SNV |
| PAH | 12 | 103238175 | 103238175 | RCV000106338 | SNV |
| PAH | 12 | 103238182 | 103238182 | RCV000000655; RCV000089195 | SNV |
| PAH | 12 | 103238202 | 103238202 | RCV000000609; RCV000089188 | SNV |
| PAH | 12 | 103238205 | 103238205 | RCV000078541 | SNV |
| PAH | 12 | 103240677 | 103240677 | RCV000000647; RCV000089177 | SNV |
| PAH | 12 | 103240687 | 103240687 | RCV000078539 | SNV |
| PAH | 12 | 103240710 | 103240710 | RCV000000608; RCV000089164 | SNV |
| PAH | 12 | 103240710 | 103240711 | RCV000106378 | deletion |
| PAH | 12 | 103240716 | 103240716 | RCV000078538 | SNV |
| PAH | 12 | 103240726 | 103240726 | RCV000000649; RCV000089157 | SNV |
| PAH | 12 | 103240726 | 103240726 | RCV000106377 | deletion |
| PAH | 12 | 103245464 | 103245464 | RCV000078537 | SNV |
| PAH | 12 | 103245479 | 103245479 | RCV000078536 | SNV |
| PAH | 12 | 103245481 | 103245481 | RCV000000644; RCV000089148 | SNV |
| PAH | 12 | 103245487 | 103245487 | RCV000078535 | SNV |
| PAH | 12 | 103245490 | 103245490 | RCV000106372 | SNV |
| PAH | 12 | 103245508 | 103245508 | RCV000106371 | SNV |
| PAH | 12 | 103246593 | 103246593 | RCV000000620; RCV000078534 | SNV |
| PAH | 12 | 103246594 | 103246594 | RCV000106370 | SNV |
| PAH | 12 | 103246597 | 103246597 | RCV000000610; RCV000078532 | SNV |
| PAH | 12 | 103246598 | 103246598 | RCV000106369 | deletion |
| PAH | 12 | 103246606 | 103246606 | RCV000000634; RCV000078531 | SNV |
| PAH | 12 | 103246617 | 103246617 | RCV000000629; RCV000089111 | SNV |
| PAH | 12 | 103246621 | 103246621 | RCV000000627; RCV000089110 | SNV |
| PAH | 12 | 103246623 | 103246623 | RCV000106368 | SNV |
| PAH | 12 | 103246639 | 103246639 | RCV000106367 | SNV |
| PAH | 12 | 103246650 | 103246650 | RCV000106366 | SNV |
| PAH | 12 | 103246653 | 103246653 | RCV000000612; RCV000078530 | SNV |
| PAH | 12 | 103246654 | 103246654 | RCV000000641; RCV000089090 | SNV |
| PAH | 12 | 103246654 | 103246654 | RCV000119826 | SNV |
| PAH | 12 | 103246659 | 103246659 | RCV000000633; RCV000089089 | SNV |
| PAH | 12 | 103246671 | 103246671 | RCV000000631; RCV000089083 | SNV |
| PAH | 12 | 103246681 | 103246681 | RCV000000614; RCV000089079 | SNV |
| PAH | 12 | 103246701 | 103246701 | RCV000000664; RCV000089065 | SNV |
| PAH | 12 | 103246704 | 103246704 | RCV000000652; RCV000089061 | SNV |
| PAH | 12 | 103246707 | 103246707 | RCV000000622; RCV000089059 | SNV |
| PAH | 12 | 103246708 | 103246708 | RCV000000619; RCV000078528 | SNV |
| PAH | 12 | 103248938 | 103248938 | RCV000106365 | SNV |
| PAH | 12 | 103248958 | 103248958 | RCV000000640; RCV000089024 | SNV |
| PAH | 12 | 103248982 | 103248982 | RCV000078527 | SNV |
| PAH | 12 | 103248988 | 103248988 | RCV000106364 | SNV |
| PAH | 12 | 103249009 | 103249009 | RCV000000621; RCV000089007 | SNV |
| PAH | 12 | 103249029 | 103249029 | RCV000106361 | SNV |
| PAH | 12 | 103249052 | 103249052 | RCV000106360 | SNV |
| PAH | 12 | 103249071 | 103249073 | RCV000106359 | indel |
| PAH | 12 | 103249087 | 103249087 | RCV000078526 | SNV |
| PAH | 12 | 103249093 | 103249093 | RCV000000663; RCV000088974 | SNV |
| PAH | 12 | 103260375 | 103260375 | RCV000078524 | SNV |
| PAH | 12 | 103260378 | 103260378 | RCV000111461 | SNV |
| PAH | 12 | 103260379 | 103260379 | RCV000106358 | SNV |
| PAH | 12 | 103260410 | 103260410 | RCV000000618; RCV000078522 | SNV |
| PAH | 12 | 103288515 | 103288515 | RCV000106354 | SNV |
| PAH | 12 | 103288515 | 103288515 | RCV000106355 | deletion |
| PAH | 12 | 103288534 | 103288534 | RCV000000611; RCV000088898 | SNV |
| PAH | 12 | 103288572 | 103288572 | RCV000000659; RCV000088892 | SNV |
| PAH | 12 | 103288579 | 103288581 | RCV000078518 | deletion |
| PAH | 12 | 103288579 | 103288581 | RCV000106353 | indel |
| PAH | 12 | 103288604 | 103288604 | RCV000000613; RCV000088884 | SNV |
| PAH | 12 | 103288638 | 103288638 | RCV000000671; RCV000088878 | SNV |
| PAH | 12 | 103288655 | 103288660 | RCV000106352 | duplication |
| PAH | 12 | 103288661 | 103288661 | RCV000078517 | SNV |
| PAH | 12 | 103288669 | 103288669 | RCV000106351 | SNV |
| PAH | 12 | 103288671 | 103288671 | RCV000000668; RCV000078516 | SNV |
| PAH | 12 | 103288682 | 103288682 | RCV000106350 | SNV |
| PAH | 12 | 103288696 | 103288696 | RCV000106349 | SNV |
| PAH | 12 | 103306572 | 103306572 | RCV000078512 | SNV |
| PAH | 12 | 103306572 | 103306572 | RCV000078513 | deletion |
| PAH | 12 | 103306573 | 103306573 | RCV000106347 | SNV |
| PAH | 12 | 103306594 | 103306594 | RCV000000639; RCV000078511 | SNV |
| PAH | 12 | 103306597 | 103306597 | RCV000000662; RCV000088839 | SNV |
| PAH | 12 | 103306601 | 103306601 | RCV000000661; RCV000088836 | SNV |
| PAH | 12 | 103306620 | 103306620 | RCV000000636; RCV000078504 | SNV |
| PAH | 12 | 103310906 | 103310906 | RCV000000653; RCV000088911 | SNV |
| PAH | 12 | 103310908 | 103310908 | RCV000000616; RCV000000617; | SNV |
| | | | | RCV000088868 | |
| PCBD1 | 10 | 72643730 | 72643730 | RCV000018290 | SNV |
| PCBD1 | 10 | 72643763 | 72643763 | RCV000018286 | SNV |
| PCBD1 | 10 | 72643778 | 72643778 | RCV000018287 | SNV |
| PCBD1 | 10 | 72643786 | 72643786 | RCV000018288 | SNV |
| PCCA | 13 | 100807344 | 100807344 | RCV000032110 | SNV |
| PCCA | 13 | 100861608 | 100861608 | RCV000032111 | SNV |
| PCCA | 13 | 100920985 | 100920985 | RCV000012803 | SNV |
| PCCA | 13 | 100920985 | 100920985 | RCV000032112 | SNV |
| PCCA | 13 | 100925472 | 100925472 | RCV000032113; RCV000078552 | SNV |
| PCCA | 13 | 100925558 | 100925558 | RCV000078546 | duplication |
| PCCA | 13 | 100953766 | 100953766 | RCV000012804 | SNV |
| PCCA | 13 | 101020767 | 101020767 | RCV000032109 | SNV |
| PCCB | 3 | 135969219 | 135969219 | RCV000079093 | SNV |
| PCCB | 3 | 135969220 | 135969220 | RCV000079095 | SNV |
| PCCB | 3 | 135974794 | 135974794 | RCV000032131 | SNV |
| PCCB | 3 | 135975428 | 135975428 | RCV000032132 | SNV |
| PCCB | 3 | 135979334 | 135979335 | RCV000079094 | indel |
| PCCB | 3 | 135980821 | 135980821 | RCV000032133 | SNV |
| PCCB | 3 | 135980866 | 135980866 | RCV000012795 | SNV |
| PCCB | 3 | 136045727 | 136045727 | RCV000012794 | duplication |
| PCCB | 3 | 136046016 | 136046029 | RCV000032124 | indel |
| PCCB | 3 | 136046026 | 136046026 | RCV000032125 | SNV |
| PCCB | 3 | 136046081 | 136046081 | RCV000012796 | SNV |
| PCCB | 3 | 136046480 | 136046480 | RCV000012798 | SNV |
| PCCB | 3 | 136047696 | 136047696 | RCV000032127 | SNV |
| PCCB | 3 | 136048782 | 136048782 | RCV000032128 | SNV |
| PCCB | 3 | 136048787 | 136048788 | RCV000012797 | insertion |
| PCCB | 3 | 136048804 | 136048804 | RCV000032129 | SNV |
| PCCB | 3 | 136048854 | 136048854 | RCV000032130; RCV000079090 | SNV |
| PTS | 11 | 112097212 | 112097212 | RCV000000506 | SNV |
| PTS | 11 | 112097240 | 112097240 | RCV000000505 | SNV |
| PTS | 11 | 112099372 | 112099372 | RCV000000511 | SNV |
| PTS | 11 | 112099388 | 112099388 | RCV000000508 | SNV |
| PTS | 11 | 112100933 | 112100933 | RCV000000510 | SNV |
| PTS | 11 | 112103901 | 112103901 | RCV000000509 | SNV |
| PTS | 11 | 112103928 | 112103928 | RCV000000513 | SNV |
| PTS | 11 | 112104187 | 112104187 | RCV000000512 | SNV |
| QDPR | 4 | 17493951 | 17493951 | RCV000000523 | SNV |
| QDPR | 4 | 17503456 | 17503456 | RCV000000520 | SNV |
| QDPR | 4 | 17506027 | 17506027 | RCV000000524 | SNV |
| QDPR | 4 | 17510986 | 17510986 | RCV000000521 | SNV |
| QDPR | 4 | 17513610 | 17513610 | RCV000000519 | SNV |
| SLC22A5 | 5 | 131705667 | 131705667 | RCV000006794 | SNV |
| SLC22A5 | 5 | 131705707 | 131705707 | RCV000006798 | SNV |
| SLC22A5 | 5 | 131705715 | 131705715 | RCV000022297; RCV000080056 | SNV |
| SLC22A5 | 5 | 131705912 | 131705912 | RCV000022308; RCV000080052 | SNV |
| SLC22A5 | 5 | 131714072 | 131714072 | RCV000006780 | SNV |
| SLC22A5 | 5 | 131714100 | 131714100 | RCV000022320; RCV000080055 | SNV |
| SLC22A5 | 5 | 131719846 | 131719846 | RCV000006791 | SNV |
| SLC22A5 | 5 | 131719847 | 131719847 | RCV000006790 | SNV |
| SLC22A5 | 5 | 131719973 | 131719973 | RCV000006789 | SNV |
| SLC22A5 | 5 | 131721062 | 131721062 | RCV000022339; RCV000080058 | SNV |
| SLC22A5 | 5 | 131721127 | 131721127 | RCV000006795; RCV000080059 | SNV |
| SLC22A5 | 5 | 131722736 | 131722736 | RCV000006785 | SNV |
| SLC22A5 | 5 | 131722737 | 131722737 | RCV000022354; RCV000080062 | SNV |
| SLC22A5 | 5 | 131724712 | 131724712 | RCV000006792 | SNV |
| SLC22A5 | 5 | 131726522 | 131726522 | RCV000022365; RCV000080047 | SNV |
| SLC22A5 | 5 | 131726524 | 131726524 | RCV000006796 | SNV |
| SLC22A5 | 5 | 131726525 | 131726525 | RCV000006793 | SNV |
| SLC22A5 | 5 | 131726531 | 131726532 | RCV000006786; RCV000080048 | duplication |
| SLC22A5 | 5 | 131728181 | 131728182 | RCV000006797 | indel |
| SLC22A5 | 5 | 131728257 | 131728257 | RCV000022379; RCV000080050 | SNV |
| SLC22A5 | 5 | 131728290 | 131728290 | RCV000006788 | SNV |
| SLC22A5 | 5 | 131729380 | 131729380 | RCV000022386; RCV000080051 | SNV |
| SLC25A13 | 7 | 95750995 | 95750995 | RCV000020704 | SNV |
| SLC25A13 | 7 | 95751007 | 95751007 | RCV000020702 | SNV |
| SLC25A13 | 7 | 95751007 | 95751007 | RCV000020703 | SNV |
| SLC25A13 | 7 | 95751008 | 95751009 | RCV000006374; RCV000006375 | duplication |
| SLC25A13 | 7 | 95751045 | 95751045 | RCV000006376 | SNV |
| SLC25A13 | 7 | 95751240 | 95751241 | RCV000006371 | duplication |
| SLC25A13 | 7 | 95751309 | 95751309 | RCV000020699 | SNV |
| SLC25A13 | 7 | 95799356 | 95799356 | RCV000006373 | SNV |
| SLC25A13 | 7 | 95813588 | 95813588 | RCV000006369; RCV000006370 | SNV |
| SLC25A13 | 7 | 95813688 | 95813688 | RCV000020698 | SNV |
| SLC25A13 | 7 | 95818685 | 95818688 | RCV000006367; RCV000006368 | deletion |
| SLC25A13 | 7 | 95820501 | 95820501 | RCV000006372 | SNV |
| SLC25A13 | 7 | 95822348 | 95822348 | RCV000020706 | SNV |
| SLC25A13 | 7 | 95822414 | 95822414 | RCV000020705 | SNV |
| SLC25A13 | 7 | 95951254 | 95951254 | RCV000020700 | SNV |
| SLC25A20 | 3 | 48895152 | 48895152 | RCV000012915 | duplication |
| SLC25A20 | 3 | 48896530 | 48896530 | RCV000012921 | SNV |
| SLC25A20 | 3 | 48897020 | 48897020 | RCV000114402 | SNV |
| SLC25A20 | 3 | 48900014 | 48900014 | RCV000012918 | SNV |
| SLC25A20 | 3 | 48921430 | 48921557 | RCV000012916 | deletion |
| SLC25A20 | 3 | 48936144 | 48936144 | RCV000012919 | deletion |
| SLC5A5 | 19 | 17983405 | 17983405 | RCV000008107 | SNV |
| SLC5A5 | 19 | 17988632 | 17988632 | RCV000008105 | SNV |
| SLC5A5 | 19 | 17988649 | 17988649 | RCV000008104 | SNV |
| SLC5A5 | 19 | 17992770 | 17992770 | RCV000008103 | SNV |
| SLC5A5 | 19 | 17992969 | 17992969 | RCV000008109 | SNV |
| SLC5A5 | 19 | 17999206 | 17999206 | RCV000008106 | SNV |
| SLC5A5 | 19 | 17999241 | 17999241 | RCV000008108 | SNV |
| TAT | 16 | 71610150 | 71610150 | RCV000000429 | SNV |
| TAZ | X | 153640466 | 153640466 | RCV000011850 | SNV |
| TAZ | X | 153640521 | 153640521 | RCV000035087 | SNV |
| TAZ | X | 153641585 | 153641585 | RCV000011859 | SNV |
| TAZ | X | 153641841 | 153641841 | RCV000035089 | SNV |
| TAZ | X | 153641844 | 153641844 | RCV000035090 | SNV |
| TAZ | X | 153641862 | 153641862 | RCV000035088 | SNV |
| TAZ | X | 153641886 | 153641886 | RCV000011857 | SNV |
| TAZ | X | 153648376 | 153648376 | RCV000011854 | SNV |
| TAZ | X | 153648377 | 153648377 | RCV000035097 | SNV |
| TAZ | X | 153648550 | 153648550 | RCV000011861 | SNV |
| TAZ | X | 153648996 | 153648996 | RCV000035098 | SNV |
| TAZ | X | 153649015 | 153649015 | RCV000029171 | SNV |
| TAZ | X | 153649015 | 153649015 | RCV000035099 | SNV |
| TAZ | X | 153649287 | 153649287 | RCV000035102 | SNV |
| TG | 8 | 133894854 | 133894854 | RCV000013532 | SNV |
| TG | 8 | 133911054 | 133911054 | RCV000013539 | SNV |
| TG | 8 | 133919031 | 133919031 | RCV000013530 | SNV |
| TG | 8 | 133935642 | 133935642 | RCV000013528 | SNV |
| TG | 8 | 133980042 | 133980042 | RCV000013541 | SNV |
| TG | 8 | 133984049 | 133984049 | RCV000013531 | SNV |
| TG | 8 | 134030185 | 134030185 | RCV000013537 | SNV |
| TG | 8 | 134034366 | 134034366 | RCV000013542 | SNV |
| TG | 8 | 134042152 | 134042152 | RCV000013540 | SNV |
| TPO | 2 | 1488368 | 1488368 | RCV000004258 | SNV |
| TPO | 2 | 1488386 | 1488386 | RCV000004259 | SNV |
| TPO | 2 | 1491613 | 1491613 | RCV000004257 | SNV |
| TPO | 2 | 1491763 | 1491763 | RCV000004260 | SNV |
| TPO | 2 | 1497748 | 1497748 | RCV000004263 | SNV |
| TPO | 2 | 1497783 | 1497783 | RCV000004268 | SNV |
| TPO | 2 | 1499831 | 1499831 | RCV000004266 | SNV |
| TPO | 2 | 1507728 | 1507728 | RCV000004261 | SNV |
| TSHB | 1 | 115576077 | 115576077 | RCV000013522 | SNV |
| TSHB | 1 | 115576128 | 115576128 | RCV000013521 | SNV |
| TSHB | 1 | 115576636 | 115576636 | RCV000013524 | SNV |
| TSHR | 14 | 81422146 | 81422146 | RCV000006812 | SNV |
| TSHR | 14 | 81554306 | 81554306 | RCV000006808 | SNV |
| TSHR | 14 | 81558891 | 81558891 | RCV000006805 | SNV |
| TSHR | 14 | 81558907 | 81558907 | RCV000006804 | SNV |
| TSHR | 14 | 81562985 | 81562985 | RCV000006824 | SNV |
| TSHR | 14 | 81606172 | 81606172 | RCV000006823 | SNV |
| TSHR | 14 | 81609330 | 81609330 | RCV000006827; RCV000122250 | SNV |
| TSHR | 14 | 81609372 | 81609372 | RCV000006810 | SNV |
| TSHR | 14 | 81609572 | 81609572 | RCV000006814 | SNV |
| TSHR | 14 | 81609630 | 81609630 | RCV000006811 | SNV |
| TSHR | 14 | 81609693 | 81609693 | RCV000006828 | SNV |
| TSHR | 14 | 81609760 | 81609760 | RCV000006806 | SNV |
| TSHR | 14 | 81609802 | 81609802 | RCV000006830 | SNV |
| TSHR | 14 | 81609832 | 81609832 | RCV000006826 | SNV |
| TSHR | 14 | 81609916 | 81609916 | RCV000006820 | SNV |
| TSHR | 14 | 81609928 | 81609928 | RCV000006818 | SNV |
| TSHR | 14 | 81609977 | 81609977 | RCV000006813 | SNV |
| TSHR | 14 | 81610039 | 81610039 | RCV000006809 | SNV |
| TSHR | 14 | 81610059 | 81610059 | RCV000006815 | SNV |
| TSHR | 14 | 81610200 | 81610200 | RCV000006829 | SNV |
| TSHR | 14 | 81610289 | 81610289 | RCV000006822; RCV000006821 | SNV |
| TSHR | 14 | 81610293 | 81610293 | RCV000006803; RCV000006802 | SNV |
| TSHR | 14 | 81610299 | 81610299 | RCV000006807 | SNV |
| TSHR | 14 | 81610317 | 81610317 | RCV000006825 | SNV |
| TSHR | 14 | 81610417 | 81610417 | RCV000006819 | SNV |
| PAX8 | 2 | 114002071 | 114002071 | RCV000014793 | SNV |
| PAX8 | 2 | 114004337 | 114004337 | RCV000014795 | SNV |
| PAX8 | 2 | 114004352 | 114004352 | RCV000014796 | SNV |
| PAX8 | 2 | 114004362 | 114004362 | RCV000014797 | SNV |
| PAX8 | 2 | 114004379 | 114004379 | RCV000014799 | SNV |
| PAX8 | 2 | 114004403 | 114004403 | RCV000014798 | SNV |
| PAX8 | 2 | 114004430 | 114004430 | RCV000014794 | SNV |
| SLC26A1 | 4 | 980918 | 980929 | RCV000078393 | deletion |
| ARG1 | 6 | 131897806 | 131897806 | RCV000002498 | SNV |
| ARG1 | 6 | 131902418 | 131902418 | RCV000002491 | SNV |
| ARG1 | 6 | 131902466 | 131902466 | RCV000002495 | SNV |
| ARG1 | 6 | 131904532 | 131904532 | RCV000002492 | SNV |
| ARG1 | 6 | 131904948 | 131904948 | RCV000002490 | SNV |
| ARG1 | 6 | 131904950 | 131904950 | RCV000002489 | SNV |
| TAT | 16 | 71602163 | 71602163 | RCV000000431 | SNV |
| TAT | 16 | 71603797 | 71603797 | RCV000000432 | SNV |
| TAT | 16 | 71606127 | 71606127 | RCV000000430 | SNV |
| GCDH | 19 | 13010285 | 13010285 | RCV000002165 | SNV |
| GCDH | 19 | 13010300 | 13010300 | RCV000002163 | SNV |
| GLA | X | 100652859 | 100652859 | RCV000011518 | SNV |
| GLA | X | 100652895 | 100652895 | RCV000011493 | SNV |
| GLA | X | 100652992 | 100652992 | RCV000011514 | SNV |
| GLA | X | 100653006 | 100653006 | RCV000011492 | SNV |
| GLA | X | 100653021 | 100653021 | RCV000011459; RCV000035299 | SNV |
| GLA | X | 100653053 | 100653054 | RCV000078263 | deletion |
| GLA | X | 100653062 | 100653062 | RCV000011490; RCV000078262 | SNV |
| GLA | X | 100653063 | 100653063 | RCV000011491; RCV000078261 | SNV |
| GLA | X | 100653067 | 100653067 | RCV000011489 | SNV |
| GLA | X | 100653067 | 100653068 | RCV000078259 | insertion |
| GLA | X | 100653358 | 100653361 | RCV000078308 | deletion |
| GLA | X | 100653374 | 100653374 | RCV000011488; RCV000078307 | SNV |
| GLA | X | 100653375 | 100653375 | RCV000011469 | SNV |
| GLA | X | 100653378 | 100653378 | RCV000011487 | SNV |
| GLA | X | 100653383 | 100653383 | RCV000078306 | SNV |
| GLA | X | 100653384 | 100653384 | RCV000078305 | SNV |
| GLA | X | 100653391 | 100653391 | RCV000078304 | SNV |
| GLA | X | 100653395 | 100653398 | RCV000078303 | deletion |
| GLA | X | 100653420 | 100653420 | RCV000029944; RCV000011486; | SNV |
| | | | | RCV000035314 | |
| GLA | X | 100653455 | 100653455 | RCV000011462; RCV000011461; | SNV |
| | | | | RCV000078302 | |
| GLA | X | 100653456 | 100653456 | RCV000078301 | SNV |
| GLA | X | 100653458 | 100653458 | RCV000078300 | SNV |
| GLA | X | 100653467 | 100653467 | RCV000011484 | SNV |
| GLA | X | 100653469 | 100653469 | RCV000011510 | SNV |
| GLA | X | 100653471 | 100653471 | RCV000011464 | SNV |
| GLA | X | 100653492 | 100653492 | RCV000078299 | SNV |
| GLA | X | 100653496 | 100653496 | RCV000011483 | SNV |
| GLA | X | 100653522 | 100653522 | RCV000011468 | SNV |
| GLA | X | 100653534 | 100653534 | RCV000078298 | SNV |
| GLA | X | 100653542 | 100653542 | RCV000011521 | SNV |
| GLA | X | 100653551 | 100653551 | RCV000011482 | SNV |
| GLA | X | 100653777 | 100653777 | RCV000011481 | SNV |
| GLA | X | 100653783 | 100653783 | RCV000011480 | SNV |
| GLA | X | 100653826 | 100653826 | RCV000078297 | SNV |
| GLA | X | 100653840 | 100653840 | RCV000078295 | SNV |
| GLA | X | 100653850 | 100653850 | RCV000035310 | SNV |
| GLA | X | 100653894 | 100653894 | RCV000011478; RCV000078294 | SNV |
| GLA | X | 100653895 | 100653895 | RCV000011479; RCV000078293 | SNV |
| GLA | X | 100653897 | 100653897 | RCV000078292 | SNV |
| GLA | X | 100653908 | 100653908 | RCV000011517 | SNV |
| GLA | X | 100653911 | 100653912 | RCV000078291 | deletion |
| GLA | X | 100653927 | 100653927 | RCV000078290 | SNV |
| GLA | X | 100653930 | 100653930 | RCV000011477; RCV000078289 | SNV |
| GLA | X | 100655663 | 100655663 | RCV000078285 | deletion |
| GLA | X | 100655687 | 100655687 | RCV000011476 | SNV |
| GLA | X | 100655745 | 100655745 | RCV000078283 | SNV |
| GLA | X | 100656658 | 100656658 | RCV000078281 | SNV |
| GLA | X | 100656683 | 100656683 | RCV000011475 | SNV |
| GLA | X | 100656701 | 100656701 | RCV000011474 | SNV |
| GLA | X | 100656731 | 100656731 | RCV000011473 | SNV |
| GLA | X | 100656740 | 100656740 | RCV000011495; RCV000078279 | SNV |
| GLA | X | 100656740 | 100656740 | RCV000011516; RCV000078280 | SNV |
| GLA | X | 100658816 | 100658816 | RCV000078277; RCV000035303 | SNV |
| GLA | X | 100658833 | 100658833 | RCV000078276 | SNV |
| GLA | X | 100658834 | 100658834 | RCV000011470; RCV000078275 | SNV |
| GLA | X | 100658834 | 100658834 | RCV000011470; RCV000078275 | SNV |
| GLA | X | 100658887 | 100658887 | RCV000078274 | SNV |
| GLA | X | 100658926 | 100658926 | RCV000078273 | SNV |
| GLA | X | 100658972 | 100658972 | RCV000011470 | SNV |
| GLA | X | 100662698 | 100662698 | RCV000011513 | SNV |
| GLA | X | 100662726 | 100662726 | RCV000011472 | SNV |
| GLA | X | 100662746 | 100662746 | RCV000078270 | SNV |
| GLA | X | 100662755 | 100662755 | RCV000078268 | SNV |
| GLA | X | 100662761 | 100662761 | RCV000011463 | SNV |
| GLA | X | 100662767 | 100662767 | RCV000078266 | SNV |
| GLA | X | 100662773 | 100662773 | RCV000078264 | SNV |
| GLA | X | 100662774 | 100662774 | RCV000011466 | SNV |
| GLA | X | 100662791 | 100662791 | RCV000011471; RCV000078260 | SNV |
| GLA | X | 100662834 | 100662834 | RCV000011511 | SNV |
| GLA | X | 100662873 | 100662873 | RCV000078272 | SNV |

### EXAMPLES

The following examples are included for illustrative purposes only and are not intended to limit the scope of the invention. Various modifications or changes suggested to persons skilled in the art are to be included within the spirit and purview of this application and scope of the appended claims.

### Example 1

### Procedure for the Receipt of Testing Request, Dried Blood Spot Shipment, and Receipt of Samples

Provided herein is an example protocol for the receipt of sample testing request, shipment of dried blood spot (DBS) collection kits, and receipt of DBS specimens at the laboratory. The collected DBS specimens provide an easy and inexpensive way to collect and store peripheral blood specimens from infants for newborn screening (NBS) test. DBS are prepared, for example, by applying a small amount of peripheral blood collected from infant heel punctures to filter paper cards, or by applying a small amount of blood collected from adult finger pricks. DBS can also be prepared, for example, from coagulated whole blood. DBS specimens are shipped at ambient temperatures as non-hazardous goods. Newborn screening (NBS) test requests are received from individual customers and healthcare facilities (hospitals, clinics, etc.).

Preparing the Test Request Receipt:

Upon receipt of a NBS test request, the record customer and/or facility contact information is recorded in a client access database. The information that is added to the client account access database includes, for example, date of the test request, name of the customer and/or facility requesting the test, name, address, phone, fax, email and preferred contact method of the customer and/or facility requesting the test. The sections with no provided information are labeled not applicable (N/A). If the sections with no provided information include sections which require pertinent information, contact is made with the customer and/or facility requesting the test, and information is obtained prior to sample processing.

Shipping the DBS Collection Kit:

The DBS collection kit is prepared for shipment to a customer and/or hospital facility requesting the test by including, for example, a standard mailing envelope or box, pre-paid, addresses outbound return envelope, blood lancet, Whatman 903 specimen collection paper, resealable impermeable plastic bag, humidity indicator cards, glassine envelope, dessicant packs, BG DBS collection instructions (exemplified in Table 15). The filter paper are identified by unique ID numbers, and the ID number for the filter paper included in the shipped DBS collection kit is recorded in the client account access database. The date of shipment of the DBS kit and the tracking number for the DBS kit are also recorded in the client access database.

Collection of DBS Specimen:

The customer is responsible for collecting the DBS specimen. Recommendations for DBS collection are provided in the DBS collection kit. The customer will complete all applicable information on the DBS submission form (attached to the Specimen Collection Paper). The DBS collection is performed, whenever possible, by a healthcare provider of a facility, and performed per the facilities standard protocols.

Shipping of the Collected DBS Specimen:

The DBS card, with attached submission form, is properly dried, and placed in the provided glassine envelope. The glassine envelope, along with desiccant packets and humidity card, are placed into the provided sealable plastic bag and the plastic bag is sealed tightly. The tightly sealed plastic bag is then to be inserted into the provided outbound envelope and immediately shipped back to the testing facility.

Receipt of Collected DBS Sample Shipment:

Upon receipt of the DBS sample at a testing facility, the date and time of sample receipt are recorded in the client account access database. The access database automatically assigns each client sample with a unique sample identification number, Sample ID, based on the date of sample receipt and the number of samples received that day (BG-MMDDYYYY-#), for example a third sample received on Mar. 28, 2014 will be assigned an ID as BG-03282014-3. The sample ID is then recorded in the data tracking system. The information that are required to be entered into the client account access database include, for example, client name and/or ID, date of sample collection, client date of birth (DOB), client age (hours), client gender, date, and time of sample receipt. Additional information provided on the DBS card is useful, and may optionally be entered into the client account access database. Sections, on the submission form, for which no data are provided are entered as not applicable (N/A) into the client account access database.

Storage of DBS Sample:

After entering the sample data into the client account access database, blood samples are placed in a 4° C. refrigerator in a laboratory, until additional sample processing, as per Example 2, commences.

### Example 2

### Procedure for gDNA Extraction, Purification, Quantification, and Normalization from Dried Blood Spots (DBS)

Provided herein is an example protocol for the extraction, purification, quantification, and normalization of gDNA from dried blood spot (DBS) samples. DBS samples are collected at an external location and shipped to the testing facility for genetic screening, according to the protocol described in Example 1. Following sample receipt, gDNA is extracted and purified from the DBS. The CHARGESWITCH nucleic acid purification kit is a bead-based technology that alters the reaction pH to facilitate nucleic acid purification. Information is collected during sample processing and stored in the laboratory information management system (LIMS).

Decontamination and Preparation for gDNA Extraction:

Before beginning the gDNA extraction process, the bench top and any necessary equipment is decontaminated with decontamination reagent to void the area of RNAase and DNA. Gloves are lightly sprayed with decontamination reagent. Three nuclease free centrifuge tubes are removed for every sample that is processed. The tubes are labeled with the sample ID, the date, and 1, 2, or 3. An additional set of tubes are labeled as “Blank 1, 2, or 3 to serve as a negative control. There are, for example, five circles for blood deposit on each DBS collection card. At least 15, 4 mm fully saturated punches are used for each extraction reaction. At least 15, 4 mm fully saturated pouches are used as negative control. An unused filter paper is processed alongside the client samples, as a “blank control” sample.

DNA Extraction Process:

DNA is extracted using a nucleic acid purification kit (e.g., CHARGESWITCH nucleic acid purification kit (Life Technologies)). A water bath is heated to 95±5° C. The water bath temperature is also recorded in the LIMS. A 1 mL volume of CHARGESWITCH lysis buffer and 10 μL of proteinase K (stored in a refrigerator) are added to Tube 1. A total of 15 fully saturated circles, are punched from the DBS sample filter paper, with a 4 mm punching device, directly into the tube labeled as Tube 1. If multiple samples are prepared for DNA extraction, the punching device is cleaned between each sample by punching a clean filter paper at least three times. A total of 15 fully saturated circles, are also punched from the Blank filter paper, with a 4 mm punching device, directly into the tube labeled as Blank #1. The tube lids are tightly secured and vortexed briefly for a short period of time, for example, 2-3 seconds. The tubes are then placed in a float tray and the float tray is put into the 95° C. water bath. The lid of the water bath is kept off during incubation to avoid contamination due to condensation. The water bath temperature fluctuates (±10° C.) during sample incubation when the lid is removed from the water bath. The tubes are incubated in the water bath for 30 minutes, with mixing by brief 2-3 seconds vortexing, every 10 minutes. During incubation, the QUBIT dsDNA High Sensitivity Standards #1 and #2 are removed from the refrigerator to equilibrate to room temperature. These are used in gDNA quantification via QUBIT as described in Example 3.

DNA Purification:

The sample(s) are removed from the water bath, following lysis, and centrifuged briefly. The serial numbers of pipettes which are used for the DNA purification procedure are recorded in the LIMS. A 200 μL volume of CHARGESWITCH purification buffer, is added to the tube labeled as Tube 2. The supernatant from the lysis reaction, as described in the DNA Extraction process section, is transferred to Tube 2. The filter paper is not transferred to Tube 2. The CHARGESWITCH magnetic beads, are vortexed for a brief time, for example 15 seconds, to thoroughly resuspend. A 20 μL volume of the magnetic beads is added to each tube labeled as Tube 2 and vortexed for a brief time, for example 2-3 seconds. The solution is incubated at room temperature for five minutes to allow DNA to bind to the magnetic beads. The tubes are placed on the magnetic rack for five minutes.

While the tube(s) remain on the magnet, the supernatant is removed and discarded. The pipette tip is angled to avoid disturbing the pellet containing the magnetic beads bound to the DNA.

DNA Wash:

The tube(s) are removed from the magnetic rack. A 500 μL volume of CHARGESWITCH wash buffer, is added to each tube labeled as Tube 2 and gently pipetted up and down to mix. The tube(s) are placed on the magnetic rack for one minute. The supernatant is removed and discarded while the tube(s) remain on the magnet. The pipette tip is angled to avoid disturbing the pellet containing the magnetic beads bound to the DNA. The wash steps, as described above, are repeated once more for a total of two washes.

DNA Elution:

The tube(s) are removed from the magnetic rack. A 150 μL volume of CHARGESWITCH elution buffer is added to each tube labeled as Tube 2 and gently pipetted up and down, around 10 times, to re-suspend the beads. The tube(s) are incubated at room temperature for five minutes. The tube(s) are placed on the magnetic rack for one minute. The supernatant, which contains the purified gDNA from the DBS sample, is removed and carefully transferred to the tubes labeled as Tube 3 while the tube(s) labeled as Tube 2 remain on the magnet.

Quantification of gDNA:

Each sample of gDNA, extracted following the procedures described above, is quantified before normalization and entrance into the library preparation process.

The process for quantification, using a QUBIT 2.0 fluorometer is described in Example 3. The “blank control” sample is also quantified using the QUBIT 2.0 fluorometer, following the process described in Example 3. The results from quantification of the “blank control” are recorded in the LIMS. The blank result is considered acceptable if it is below the detection limit for the QUBIT 2.0 fluorometer.

### Example 3

### Procedure for gDNA Quantification Using the LIFE TECHNOLOGIES QUBIT 2.0 Fluorometer

Provided herein is an example protocol for the quantification of double stranded (ds) DNA using the QUBIT 2.0 Fluorometer and dsDNA High Sensitivity (HS) kit (Life Technologies). The dsDNA High Sensitivity Kit contains QUBIT dsDNA HS Reagent, QUBIT dsDNA HS Buffer, QUBIT dsDNA HS Standard 1, and QUBIT dsDNA HS Standard 2. The HS Reagent and buffer are stored at room temperature. The Standards 1 and 2 are stored at 4° C. HS standards are aliquoted into small aliquots to avoid cross-contamination during pipetting.

gDNA is extracted from the DBS Samples, following the protocol described in Example 2. After the extraction process, the DNA samples are quantified prior to entry into library preparation, using the QUBIT 2.0 Fluorometer. The QUBIT 2.0 Fluorometer is also used to quantify completed libraries and pools prior to sequencing, which is described in Example 5. The QUBIT fluorometer is qualified to evaluate 1-500 ng/mL gDNA concentrations. All client unknown samples are diluted within the quantifiable range.

For quantification, a working solution is prepared for dilution of all samples and standards. Each time a new working solution is created, standards are diluted and re-quantified for that specific batch of working solution. A quality control sample is also analyzed on the QUBIT prior to sample analysis. The quality control sample is prepared by diluting the HS Standard 2 to evaluate the normal reading range of the QUBIT (1-500 ng/mL). The acceptable range for the quality control sample is defined as the ±10% of the expected target concentration.

Preparation of the Working Solution:

The QUBIT DNA HS Reagent is diluted 1:200 in the QUBIT DNA HS Buffer. 200 μL working solutions are prepared for each standard and unknown sample. It is recommended to create enough working solution for N+2 to accommodate for possible assay repeats. For example, for the analysis of 10 unknown samples, a total of 2. 8 mL of working solution is prepared (2.0 mL for samples, 400 μl for standards, 200 μl for quality control sample, 200 μl for possible rerun).

Once prepared, the working solution is stable at room temperature for three hours. Due to the nature of the light-sensitive dye, the solution is stored in minimal light exposure.

Preparation of Assay Tubes:

The assay tubes are set up for each unknown sample, two standards (1 and 2), and one quality control sample.

The samples, quality control and standards tubes are prepared according to Tables 6 and 7. The unknown samples are diluted between 1:10 and 1:200 to fall within the quantifiable range of the instrument. The sample dilution varies depending on the concentration of gDNA in the unknown sample.

TABLE 6
QUBIT Assay Preparation

| | Final | Working | Sample/ | Total |
|---|---|---|---|---|
| | Dilution | Solution | Standard | Volume |
| | (μL) | (μL) | (μL) | (μL) |
| Standards | 1:20 | 190 | 10 | 200 |
| Unknown | 1:200 | 199 | 1 | 200 |
| Samples | 1:100 | 198 | 2 | 200 |
| | 1:50 | 196 | 4 | 200 |
| | 1:10 | 180 | 20 | 200 |

TABLE 7
Quality Control Sample Preparation

| Quality | Volume | Volume | Final Target | |
|---|---|---|---|---|
| Control | Standard | Working | Concentration | Acceptable QC |
| Sample | 2 (μL) | Solution (μL) | (ng/mL) | Range (ng/mL) |
| Quality | 5 | 195 | 250 | 225-275 |
| Control | | | | |

After preparing the tubes, according to dilutions exemplified in Tables 6 and 7, all the tubes are vortexed for a brief time, for example 2-3 seconds. The tubes are flicked or tapped to ensure all solution is at the bottom of the assay tube and no bubbles are present. The tubes are then incubated at room temperature for two minutes.

QUBIT Reading:

The QUBIT 2.0 fluorometer is powered on and, “DNA” and “High Sensitivity” options are selected. The outside of the tubes are cleaned with a delicate task wipe to remove any marks or debris that interferes with the reading. The standards 1 and 2 are first read followed by the quality control samples. The QUBIT reading for the quality control samples are recorded in the LIMS. It is indicated that the reading is within the defined acceptable range for the instrument. If the quality control sample is not within the defined acceptable range, a new working solution and dilutions must be prepared. The unknown samples are read after reading the quality control samples. If the sample quantification value is outside of the QUBIT HS range (high or low), the sample is re-diluted and the reading is repeated. The sample quantification value (in ng/mL) is recorded in the LIMS.

Concentration Adjustment and Calculation:

The final stock concentration of each unknown sample is calculated using the following formulae:

QUBIT reading value (QF value) is multiplied by the working solution dilution factor, as follows:

$$1 : 100 \text{Dilution:} Sample Concentration ( \frac{ng}{mL} ) = QF Value \times 100$$

The value is converted to ng/mL, as follows:

$$Sample Concentration ( \frac{ng}{µL} ) = \frac{Sample Conc ( \frac{ng}{mL} )}{1000}$$

The total gDNA concentration is calculated by multiplying the ng/μL value by the volume used to elute the DNA during the DNA extraction process.

$$150 µL Elution Volume \text{:} Total gDNA ( ng ) = Sample concentration ( \frac{ng}{µL} ) \times 150$$

Maintenance and Storage:

After the reading, the QUBIT is stored in an area with no direct sunlight. The machine is unplugged and cleaned gently with an alcohol swab or delicate task dampened with 20% ethanol solution. It is indicated on Appendix 1, that the cleaning is performed after use.

### Example 4

### Normalizing gDNA for Library Preparation

Provided herein is an example protocol for normalizing the gDNA concentrations prior to library preparation. All samples entering library preparation should be normalized to a concentration of 2 ng/μL.

Normalizing gDNA for Library Preparation:

An appropriate amount of 10 mM tris-HCL is added to each gDNA sample to obtain the desired concentration using the following formula:

$$gDN A Concentration ( \frac{ng}{µL} ) = \frac{( Qubit Value ( \frac{ng}{mL} ) \times Qubit Dilution Factor )}{1000}$$

$$Final Sample Volume ( µL ) = \frac{( gDNA Concentration ( \frac{ng}{µL} ) \times Sample Volume ( µL )}{2}$$

$$Tris Volume ( µL ) = Final Sample Volume ( µL ) - Sample Volume ( µL )$$

Samples, at normalized concentrations, are used for the library preparation process, performed in Laboratory 2, as described in Example 5. The samples are stored at −20° C. in the freezer.

### Example 5

### Procedure for Library Preparation

Provided herein is an example protocol for the preparation of sequencing library from genomic DNA using an anchored multiplex PCR (AMP) technology (e.g., ENZYMATICS).

Client Samples:

The extracted, quantified, and normalized gDNA samples, prepared following methods described in Examples 2-4 enter the library preparation process which includes the following steps: DNA fragmentation, A-tailing and end repair, adapter ligation, and amplification via PCR steps. The technology used for library preparation is the lyophilized version of ARCHER DNA assay kit.

Quality Control (QC) Samples:

A library preparation QC sample is analyzed no less often than quarterly and whenever a reagent lot number changes, to verify the accuracy of the genomic analysis. A QC sample contains, for example, at least one known variant (within the target gene panel) to be considered a positive control. A QC sample does not display, for example, a known variant (within the target gene panel) to be considered a negative control. A single sample serves as both the positive and negative control if donor sample exhibits a known variant in a target gene and does not display a variant in another target region.

QC samples are obtained from donors with known genomic variants within the target gene panel. In some embodiments, QC samples are derived from the Coriell Institute. In some embodiments, QC samples are derived from the triplicate analysis of unknown samples for the development of a known set of variants within the target gene panel.

QC samples are diluted with 10 mM tris HCl to a final concentration of 2 ng/μL.

$$Final Sample Volume ( µL ) = \frac{( gDNA Concentration ( \frac{ng}{µL} ) \times Sample Volume ( µL )}{2}$$

$$Tris Volume ( µL ) = Final Sample Volume ( µL ) - Sample Volume ( µL )$$

Diluted samples are aliquoted into cryogenic vials and stored at −20° C. The QC samples are processed under normal conditions with the unknown client samples.

DNA Fragmentation, performed in Laboratory 2:

The DNA Fragmentation reaction packet is removed and allowed to reach room temperature. The following thermal cycling program is started (see Table 8) as described in Example 7 and paused once the block reaches 4° C.

TABLE 8
DNA Fragmentation Incubation

| Step | Incubation Temperature | Incubation Time |
|---|---|---|
| 1 | 4° C. | 1 min |
| 2 | 37° C. | 12 min |
| 3 | 72° C. | 20 min |
| 4 | 4° C. | Hold |

Heated Lid on at 105° C.

The green 8-tube strips are removed. Each tube in the strip provides a single reaction. If necessary, the tubes are centrifuged briefly to collect all lyophilized materials at the bottom of the tube. The tubes are labeled with sample ID and placed in a bench top chiller.

The following steps are performed with a single reaction tube open at a time: The lid of the first reaction tube is opened. A 50 μL aliquot of the 2 ng/μL purified gDNA sample (prepared as described in Examples 2 and 3) into the reaction tube. Contact is avoided between the pellet and pipette tip, while dispensing the solution. The lid of the first reaction tube is closed. The process is repeated for each reaction tube. Once DNA is added to each reaction tube, the tubes are gently tapped, for example, 2-3 times to mix solutions. The tubes are briefly centrifuged to collect contents at the bottom of the tubes. The tubes are placed into the block of the paused thermal cycler and program is resumed.

Index 2 Barcode Adapter Addition:

The Index 2 adapter reaction packet (ILLUMINA) is removed along with the Adapter Ligation reaction packet, and both are allowed to reach room temperature. The INDEX 2 barcode adapter reaction packet is opened and the 8 tube-strip is removed. Each reaction contains a unique Index 2 barcode (1 through 48). It is ensured that each sample is placed into the appropriate reaction tube. If necessary, the tube strip is briefly centrifuged to collect all lyophilized materials at the bottom of the tube. The tubes are labeled with sample ID and placed in a bench top PCR tube chiller. A 50 μL aliquot of fragmented gDNA (from Laboratory 2) is transferred into the Barcode Adapter tubes. Care is taken to avoid touching the lyophilized pellet with pipette tip while dispensing solution. The tube lids are closed securely and the tubes are gently tapped 2-3 times to mix. The tubes are centrifuged briefly and returned to a bench top chiller. If all eight tubes are not utilized, the unused tube(s) are labeled with appropriate adapter number (1-8) and returned to the refrigerator.

Adapter Ligation:

The red 8-tube strip is removed from the Adapter ligation reaction packet. If necessary, the tubes are centrifuged briefly to collect all lyophilized materials at the bottom of the tube. The tubes are labeled with sample ID and placed in bench top PCR tube chiller. A 50 μL aliquot of fragmented DNA with Index 2 Barcode Adapters is transferred into the tubes containing Adapter Ligation mix. Care is taken to avoid touching the lyophilized pellet with pipette tip while dispensing solution. The lids are closed securely and the tubes are gently tapped tubes 2-3 times to mix. The tubes are centrifuged briefly and returned to a bench top chiller. The tube strip is placed in the thermal cycler and the reaction is incubated, as described in Example 7, and exemplified in Table 9.

TABLE 9
Adapter Ligation Incubation

| Step | Incubation Temperature | Incubation Time |
|---|---|---|
| 1 | 16° C. | 30 min |
| 2 | 22° C. | 30 min |
| 3 | 4° C. | Hold |

Heated Lid Off or Not Closed

Post-Adapter Ligation Purification:

It is ensured that AMPURE XP beads are at room temperature. The tubes are removed from the thermal cycler and briefly centrifuged. The samples are not placed in a bench top chiller, as this purification step occurs at room temperature. The AMPURE XP beads are vortexed for a brief time, for examples, for 15 seconds for thorough re-suspension. A 40 μL volume of AMPURE XP beads is added to each 50 μL reaction for a ratio of 0.8×. All caps are secured and the tubes are vortexed for 2-3 seconds. The tubes are incubated room temperature for 5 minutes. The tubes are placed on magnet for 4 minutes or until solution is clear. The supernatant is carefully pipetted and discarded without disturbing the beads. The beads are washed twice with 200 μL of 70% ethanol while on the magnet (the strip is moved on magnet to thoroughly wash beads). The 70% ethanol, used for washing, is freshly prepared weekly. After the second wash, it is ensured that all solution is removed from tubes and the beads are allowed to dry for 6 minutes at room temperature. The tubes are removed from the magnet and thoroughly re-suspended in 24 μL of 10 mM Tris-HCl. The 10 mM Tris-HCl is freshly prepared weekly. The tubes are placed tubes back on the magnet for 2 minutes.

If it is determined that the second wash is a stopping point, then 24 μL of purified solution is carefully transferred into new 200 μL PCR tubes and stored at −20° C. The tubes are labeled with sample ID.

If it is determined that the second wash is not a stopping point, then the lids of the tubes are securely closed and the tubes are transferred into the PCR workstation, after proper UV irradiation, to prepare for a first PCR.

First PCR:

UV light is activated for decontamination of the PCR workstation, for 15 minutes prior to PCR setup. It is ensured that all necessary supplies and equipment are present before activating UV light. DNA samples and PCR reagents are not placed into the workstation until after UV irradiation. The UV decontamination step is recorded in the LIMS.

The First PCR Reagents are removed from freezer and allowed to thaw in a bench top chiller. The enzyme (PHOENIX HS TAQ POLYMERASE) is kept in the freezer until utilized and always kept in a −20° C. bench top chiller while outside the freezer. Following UV decontamination, the laminar flow is activated. The purified DNA and the First PCR Reagents are moved into the PCR workstation. The operator changes gloves to prevent contamination in the PCR hood.

The tubes are labeled with sample ID and placed in a bench top chiller. The following reagents are added into each tube while in a bench top chiller. A PCR1 master mix is created when preparing multiple samples, as exemplified in Table 10.

TABLE 10
Master Mix preparation for First PCR

| First PCR Reagent | Volume (μL) |
|---|---|
| 5X Phoenix Buffer | 4 |
| 2 mM dNTP | 2 |
| 10 μM P5_1 | 2 |
| Primers GC-content Low GSP-1 (100 μM) | 2 |
| OR | |
| Primers GC-content High GSP-1 (100 μM) | |
| Phoenix HS Taq Polymerase | 0.4 |
| Nuclease Free Water | 0.6 |
| TOTAL VOLUME | 11 |

The tubes containing purified DNA from previous step are placed onto a magnet. A 9 μL aliquot of purified DNA is transferred from Adapter Ligation into two sets of First PCR reaction tubes (GC-content High and GC-content Low). Each set of tubes is labeled with the sample ID and GC-content. The reactions are mixed by gently pipetting up and down 10 times. The lids of the tubes are closed securely and the tubes are centrifuged briefly.

The PCR tubes are placed in the thermal cycler and the PCR reaction is incubated, as described in Example 7, and exemplified in Table 11.

TABLE 11
First PCR Incubation

| | Incubation | | |
|---|---|---|---|
| Incubation Temperature | Time | | # of cycles |
| 94° C. | 30 | sec | 1 |
| 94° C. | 30 | sec | 20 |
| 60° C. for GC-content Low samples | 90 | sec | |
| OR | | | |
| 64° C. for GC-content High samples | | | |
| (ramp rate of 2.3° C./sec) | | | |
| 72° C. | 3 | min | 1 |

| | | |
|---|---|---|
| 4° C. | HOLD | 1 |

Heated Lid on at 105° C.

After the First PCR is set up, all equipment remains in the PCR workstation for decontamination. All surfaces and equipment are wiped with RNASE Away. Any waste that leaves the workstation is put in a waste bag and the waste bag is tightly closed with a rubber band.

Post-First PCR Purification:

It is ensured that AMPURE XP beads are at room temperature. The tubes are removed from the thermal cycler and briefly centrifuged. The samples are not placed in a bench top chiller, as this purification step occurs at room temperature. The AMPURE XP beads are vortexed for a brief time, for example, for 15 seconds for thorough re-suspension. A 16 μL volume of AMPURE XP beads is added to each 20 μL First PCR reaction for a ratio of 0.8×, and the reaction mixture is pipetted 10 times to mix. The lids are secured and the tubes are incubated room temperature for 5 minutes. The tubes are placed on magnet for 4 minutes or until solution is clear. The supernatant is carefully pipetted and discarded without disturbing the beads The beads are washed twice with 200 μL of 70% ethanol while on the magnet (the strip is moved on magnet to thoroughly wash beads). The 70% ethanol, used for washing, is freshly prepared weekly. After the second wash, it is ensured that all solution is removed from tubes and the beads are allowed to dry for 5 minutes at room temperature. The tubes are removed from the magnet and thoroughly re-suspended in 9 μL of 10 mM Tris-HCl. The 10 mM Tris-HCl is freshly prepared weekly. The tubes are placed tubes back on the magnet for 2 minutes.

If it is determined that the second wash is a stopping point, then 9 μL of purified DNA solution is carefully transferred into 200 μL PCR tubes and stored at −20° C. The tubes are labeled with sample ID and GC-content Low or High.

If it is determined that the second wash is not a stopping point, then the lids of the tubes are securely closed and the tubes are transferred into the PCR workstation, after proper UV irradiation, to prepare for a second PCR.

Second PCR:

UV light is activated for decontamination of the PCR workstation, for 15 minutes prior to PCR setup. It is ensured that all necessary supplies and equipment are present before activating UV light. DNA samples and PCR reagents are not placed into the workstation until after UV irradiation.

The Second PCR Reagents are removed from freezer and allowed to thaw in a bench top chiller. The enzyme (PHOENIX HS taq polymerase) is kept in the freezer until utilized and always kept in a bench top chiller while outside the freezer. Following UV decontamination, the laminar flow is activated. The purified DNA and the Second PCR Reagents are moved into the PCR workstation. The operator changes gloves to prevent contamination in the PCR hood.

The tubes are labeled with sample ID and placed in a bench top chiller. The following reagents are added into each tube while in a bench top chiller. A master mix is created when preparing multiple samples, as exemplified in Table 12.

TABLE 12
Master Mix preparation for Second PCR

| Second PCR Reagent | Volume (μL) |
|---|---|
| 5X Phoenix Buffer | 4 |
| 2 mM dNTP | 2 |
| 10 μM P5_2 | 2 |
| Primers GC-content Low GSP-2 (100 μM) | 2 |
| OR | |
| Primers GC-content Low GSP-2 (100 μM) | |
| Phoenix HS Taq Polymerase | 0.4 |
| Nuclease Free Water | 0.6 |
| 10 μM P7 Index (#1-12) | 2 |
| TOTAL VOLUME | 11 |

The tubes containing purified DNA from previous step are placed onto a magnet. A 7 μL aliquot of purified DNA is transferred from Adapter Ligation into each GC-content Low or High Second PCR reaction tube. The reaction is mixed by gently pipetting up and down 10 times. The lids of the tubes are closed securely and the tubes are centrifuged briefly.

The GC-content Low and High PCR tubes are placed in the thermal cycler and the PCR reactions are incubated, following the procedure described in Example 7, and exemplified in Table 13.

TABLE 13
PCR 2 Incubation

| | Incubation | | |
|---|---|---|---|
| Incubation Temperature | Time | | # of cycles |
| 94° C. | 30 | sec | 1 |
| 94° C. | 30 | sec | 20 |
| 60° C. for GC-content Low samples | 90 | sec | |
| OR | | | |
| 64° C. for GC-content High samples | | | |
| (ramp rate of 2. 3° C./sec) | | | |
| 72° C. | 3 | min | 1 |

| | | |
|---|---|---|
| 4° C. | HOLD | 1 |

Heated Lid on at 105° C.

Library Normalization: Following the second PCR, the individual samples are set to the same concentration via bead normalization.

Samples are treated with Exonuclease I to remove unwanted single-stranded DNA (ssDNA) and primer extension is performed on the completed AMP libraries.

A master mix containing the following reagents is prepared:

| | VoL per | Concentration | |
|---|---|---|---|
| Component | Reaction (μL) | | |
| Protected P5/P7 capture | 0.192 | 8 | nM |
| oligonucleotides (1 μM stock, | | | |
| diluted in 1X Phoenix Taq Buffer) | | | |
| 10 mM dNTPs | 0.4 | 0.167 | mM |
| 5x Phoenix Buffer | 0.8 | 1x | |
| H2O | 0.66 | | |
| 20 units/μL Exonuclease I | 2 | 1.67 | units/μL |

4 uL of the master mix is added to each 20 uL post-second PCR reaction and incubated according to the condition listed below:

Incubation Conditions

| | |
|---|---|
| 37° C. | 30 min |
| 94° C. | 5 min |
| 55° C. | 30 min |
| 72° C. | 1 min |
| 4° C. | hold |

A buffer solution is prepared by combining the following reagents:

| | Volume to add (for 10 mL | | |
|---|---|---|---|
| Component | total Vol.) | | Working Concentration |
| 5M NaCl | 2 | mL | 1M |
| 1M Tris pH | 100 | μl | 10 mM |
| 8.0 | | | |
| 0.5M EDTA | 20 | μL | 1 mM |
| Tween | 10 | μL | 0.1% |

Following the exonuclease treatment and primer extension, the samples are removed from the thermal cycler and placed in a benchtop chiller.

To perform streptavidin bead equilibration, the magnetic beads are resuspended by thorough vortexing. 10 uL of beads are removed from resuspended bead stock and placed into an empty PCR tube. The tube containing the beads is placed on a magnet for one minute and then the supernatant is discarded. The beads are washed by suspending with 20 uL of the previously prepared buffer solution. The samples are placed on the magnet and supernantant is removed and discarded. The wash is repeated two more times for a total of three washes. Resuspend in 10 uL of the Buffer solution and allow to sit off of the magnet.

0.2M NaOH is freshly prepared and the HT1 Buffer is thawed.

Equal volumes of each library are combined into a new PCR tube. 48 μL of the combined pool is transferred into a new PCR tube. The following amounts of each sample are combined depending on the primer set utilized:

| | Vol. per High | |
|---|---|---|
| # of Libraries to pool | GC sample (μL) | Vol. per Low GC sample (μL) |
| 10 × 2 | 5.9 | 4.1 |

10 uL of the beads are added to the 48 uL of combined-sample pool and mixed well with pipetting. The mixture is incubated for 15 minutes at room temperature with intermittent mixing to resuspend the beads. Following the incubation, the tubes are briefly spun down and then placed on the magnet. After the beads migrate to the magnet, the supernatant is removed and discarded. The beads are washed on the magnet with 50 uL of the buffer solution by moving the tubes from one side of the magnet to the other. Once the beads have migrated to the magnet, the supernant is removed and discarded. The wash is repeated twice for a total of three washes.

The samples are briefly spun down and any residual supernantant is removed. The beads are resuspended in 15 uL of the freshly prepared 0.2 M NaOH off the magnet and incubated at room temperature for 10 minutes. The tubes are gently flicked one to two times during incubation to mix the beads. While sample is incubating, 185 uL of HT1 Buffer is added to a new tube.

After the 10 minute incubation, the tube containing the beads is placed on the magnet until the supernatant is clear. All 15 ul of the supernatant is removed and placed into the tube containing the HT1 Buffer. Now the normalized, denatured, and diluted pool is stored at −20±5° C. if needed.

### Example 6

### Procedure for Use of the ILLUMINA MISEQ Sequencer in an Exemplary Method

Provided herein is an example protocol for the use of the ILLUMINA MISEQ sequencer. This protocol is used to perform targeted gene sequencing on patient samples following gDNA extraction, as described in Example 2, and sample library preparation, as described in Example 5. The reagents and kits used in the protocol are, for example, the PhiX control (10 NM), the MISEQ V2 reagent kit which include a reagent cartridge (stored at −20° C.), hybridization buffer (HT1) (Stored at −20° C.), PR2 bottle (stored at 4° C.), and flow cell (stored at 4° C.).

Sample Sheet Preparation (Data Tracking System):

A fresh solution of 0.2 N NaOH is prepared to properly denature samples. Once prepared, the Nao dilution is stable for 12 hours.

PhiX Control Preparation:

The 10 NM Phi Control is thawed in a bench top chiller. The hybridization buffer (HT1) is thawed at room temperature. The HT1 is stored at 4° C. until ready to use.

PhiX Denaturation:

A 4 nM dilution of PhiX control is prepared by combining the following, 10 nM PhiX Control (2 μL), 10 mM Tris-HCl, pH 8.0 with 0.1% tween 20 (3 μL). The solution is pipette up and down to mix. The following are combined to denature the 4 nM PhiX Control, 4 nM PhiX Control (5 μL), 0.2 N NaOH (5 μL), Vortex to mix and centrifuge briefly. The mixture is incubated at room temperature for 5 minutes to denature. A 20 pM dilution of denatured PhiX is prepared by combining the following, HT1 (990 μL), 4 nM denatured PhiX Control (10 uL). This 20 pM PhiX stock is stored at −20° C. for up to 21 days for multiple uses.

If performing a MISEQ sequencer Run: A 11-14 pM dilution of denatured PhiX is prepared by combining the following, HT1 (45 μL) and 20 pM denatured PhiX Control (45 μL). The final volume of PhiX dilution should be 90 μL.

Reagent Cartridge Thaw:

After the library pool is equilibrated to a 2-6 nM solution as described in Example 5, the reagent cartridge (MISEQ sequencer V2 Reagent Kit Box 1 of 2) is thawed in a room temperature water bath for one hour. Care is taken to ensure that he water bath level does not exceed the “max fill” mark indicated on the side of the cartridge. If the reagent cartridge is thawed prior to library preparation completion, the reagent cartridge can be placed at 4° C. for up to 24 hours.

Final Library Pool Preparation:

The tubes are vortexed to mix and centrifuged briefly.

Preparation of 11-14 pM Library: A 600 μL volume of diluted library, with a concentration of 11-14 pM, is prepared by adding an appropriate volume of hybridization buffer (HT1) to dilute, according to the following equation:

$$Volume of 13 pM Library ( µL ) = \frac{( 600 µL ) ( 13 pM )}{Denatured Pool Conc . ( pM )}$$

Preparation of 11-14 pM Library with 15% PhiX: A diluted library, with a concentration of 11-14 pM, is prepared by combining the following, 11-14 pM Library (510 μL, 10 pM denatured PhiX control (90 μL. The PhiX control is freshly diluted to 11-14 pM. The final dilution of the PhiX control must equal the final library concentration.

The tubes are vortexed to mix and microcentrifuged briefly.

Loading Reagent Cartridge:

After cartridge reagents have thawed, they are slowly inverted 10 times to mix. The foil of well 17 is punctured with a new pipette tip, in preparation for library loading. 600 μL of the 11-14 pM Library with 15% PhiX is loaded into the well. The cartridge is firmly tapped on the table to ensure all air pockets are removed from the bottom of the cartridge wells.

Starting a MISEQ Sequencer Run:

The MISEQ control software is initiated and the Welcome screen appears. The “Sequence” option is selected to proceed with. Appropriate account information is used for logging in for Base space monitoring.

Preparation of the Flowcell:

The flowcell is removed (Box 2 of 2) and rinsed thoroughly with nuclease-free water. All moisture on the plastic of the flowcell is removed delicate task wipe. The glass of the flow cell is cleaned using an alcohol wipe or 70% ethanol solution. Care is taken to avoid leaving residue or streaks. It is recommended to use only lens paper on the glass of the flow cell to avoid any damage to the flow cell surface. Before loading the flow cell, the flow cell stage is briefly wiped with an alcohol wipe or 70% ethanol. Ensure no excess moisture, streaks or debris is present between the lanes of the flow cells before loading it onto the stage of the MISEQ sequencer. The flow cell is loaded carefully the cover is gently latched before closing the flow cell compartment door.

Loading PR2 Wash Solution:

The wash solution bottle in the left position of the reagent chiller is removed and replaced it with the new PR2 bottle (Box 2 of 2). The waste container is emptied prior to starting each individual run. Liquid waste is collected in the waste container is disposed of in a liquid hazardous waste container due to the presence of formamide in Well 8 of the MISEQ V2 cartridge. It is ensured that the sippers are lowered into the appropriate container and have no obstructions.

Loading the Reagent Cartridge:

The wash cartridge from the MISEQ sequencer is removed and replaced with the recently loaded reagent cartridge.

Sample Sheet Recognition:

If the sample sheet is saved correctly in the designated folder, the software recognizes and uploads the information. If the sample sheet is not recognized it is due to improper sample sheet naming, wrong location of the saved sample sheet, or formatting error of the sample sheet.

Pre-Run Check:

Once the sample sheet is loaded, the MISEQ sequencer automatically begins a pre-run check. Once the pre-run check is successfully completed, the next step is to proceed with “Sequence” to begin the run. The date of the sequencing run is recorded on Appendix 1.

Post-Run Wash:

The post-run wash is the standard instrument wash performed between sequencing runs and consists of a single wash cycle. The instrument automatically prompts the user to perform a post-run wash using the following steps: a 10% tween solution is prepared by combining, 5 mL 100% tween 20 (5 mL, lab grade water (45 mL, a 0.5% tween solution by combining, 10% tween solution (25 mL, lab grade water (475 mL). The 0.5% tween solution is added to each reservoir of the wash tray. A 50 mL volume of 0.5% tween solution is added to the modified wash solution bottle. When the sequencing run is complete, “Start Wash” is selected to initiate the post-wash run. The wash tray and modified wash solution bottle are inserted. It is ensured that the waste bottle is empty. (see hazardous waste warning, above). The subsequent step is to select “Next” to begin the post-wash run. The date of the post-wash run is recorded on Appendix 1.

### Example 7

### Procedure for the APPLIED BIOSYSTEMS VERITI Thermal Cycler

Provided herein is an example protocol for the general operation, maintenance, and calibration of the APPLIED BIOSYSTEMS VERITI thermal cycler. The VERITI thermal cycler is used during the sample library preparation process, as described in Example 5. The VERITI thermal cycler is used for DNA fragmentation, adapter ligation, as well as the first and second PCR processes. The library preparation process, including use of the Veriti system, is internally validated during the MISEQ sequencer instrument validation.

Performing a Run:

Samples are prepared per the sample preparation protocol, described in Example 5, and placed in a bench top cooler. The cover is closed and Browse/New Methods are touched. The desired run method is located and selected. The reaction volume is edited and/or cover temperature, if necessary. It is ensured that the cover temperature is properly heated to the desired temperature prior to loading samples into the thermal cycler. The unique Run ID is entered. A run is initiated by pressing by pressing Start Run Now. The sample temperature is monitored until it reaches the desired Stage 1 temperature. For example: If Stage 1 requires a temperature of 95° C., monitor the sample temperature until the desired temperature is reached. When desired temperature is reached, the prepared sample tubes from the bench top chiller are immediately loaded into sample block. If a single sample tube strip is processed, an empty tube strip is inserted next to the sample tube strip to ensure that the thermal cycle cover does not damage the sample tube strip.

Monitoring a Run:

The run screen displays run details including the temperature, time, and current run stage. A status report which is displayed at the bottom of the screen displays any errors that occurred during the run. If necessary, the run is paused or stopped.

Run Report:

The run report is displayed at the end of the run. The run report is saved until the next run is finished and must be saved or printed if documentation is required.

### Example 8

### Validation Plan for the ILLUMINA MISEQ Sequencer Instrument

Provided herein is an example validation plan to validate the performance of the ILLUMINA MISEQ sequencer instrument to identify genomic variants in human DNA samples in comparison to a reference genome.

For Purposes of the Validation Plan:

False Negative (FN) is defined as Negative result (no variant found) when test is positive (variant present).

False Positive (FP) is defined as Positive result (variant found) when test is negative (no variant is present).

Precision is defined as the closeness of agreement between values obtained by replicate measurements on the same or similar objects under specified conditions.

Within-Run Variability (Repeatability) is defined as the degree to which the same sequence is derived when sequencing the same reference sample many times under the same conditions.

Between-Run (Reproducibility) is defined as the degree to which the same sequence is derived when sequencing the same reference sample many times under variable conditions (multiple days and/or multiple operators).

Sensitivity is defined as the ability to detect all confirmed variants in a sample (true test result) if the variant is present.

Specificity is defined as the proportion of samples that have a negative test result when no variant is present.

True Negative (TN) is defined as Negative result (no variant found) when test is negative (variant not present).

True Positive (TP) is defined as Positive result (variant found) when test is positive (variant is present).

TABLE 14
Known Sample Panel (Coriell Institute)

| | | | | Coriell |
|---|---|---|---|---|
| | Targeted | | | Institute |
| | Gene | Condition/Disease | Gene Mutation | ID |
| 1 | ACADM | Medium-Chain Deficiency of Acyl- | 985A > G/Lys304Glu | NA07523 |
| | | CoA Dehydrogenase | | |
| 2 | ACADM | Medium-Chain Deficiency of Acyl- | c. 166G > C/Ala56Pro | NA11319 |
| | | CoA Dehydrogenase | | |
| 3 | ACADS | Short-Chain Deficiency of Acyl- | 319C > T/Arg107Cys | NA17470 |
| | | CoA Dehydrogenase | | |
| 4 | BCKDHA | Maple Syrup Urine Disease Type | 8-bp deletion in exon 7 | NA00651 |
| | (BCKAD) | 1A | | |
| 5 | CFTR | Cystic Fibrosis | c. 1811G > C/Arg560Thr | NA11284 |
| | | | Phe508Del | |
| 6 | CFTR | Cystic Fibrosis | c. 1496C > A/Ala455Glu | NA08211 |
| | | | Phe508Del | |
| 7 | CFTR | Cystic Fibrosis | Phe508DEL | NA00897 |
| | | | c. 1172C > G/Arg347Pro | |
| 8 | CYP21A2 | Adrenal Hyperplasia | 30kb deletion of CYP21A2 | NA14734 |
| | | | c. 292G > C | |
| 9 | GALT | Galactosemia - not affected | Asp98His | NA01212 |
| 10 | GALT | Galactosemia | c. 2744A > G Asn314Asp | NA01996 |
| 11 | GALT | Galactosemia | c.563A > G/Gln188Arg | NA00422 |
| | | | c. 1030C > A/Gln344Lys | |
| 12 | GALT | Galactosemia | c. 855G > T/Lys285Asn | NA00149 |
| 13 | GBA | Gaucher | 1448T> C/Leu444Pro | NA10915 |
| 14 | GBA | Gaucher | c. 1226A > G/Asn370Ser | NA00372 |
| | | | 84GG | |
| 15 | GLA | Fabry | c. 485G > A/Trp162Ter | NA00107 |
| 16 | GLA | Fabry | c. 658C > T | NA00881 |
| 17 | NPC1 | Niemann-Pick C1 | c. 709C > T/Pro237Ser | NA03124 |
| | | | c. 3182T > C/Ile1061Thr | |
| 18 | PAH | Phenylketonuria | 896T > G/Phe299Cys | NA02406 |
| | | | 1222C > T/Arg408Trp | |
| 19 | PAH | Phenylketonuria | c. 755G > A/Arg252Gln | NA01565 |
| 20 | SLC22A5 | Primary Carnitine Deficiency | No mutations in SLC22A5 | NA10665 |
| 21 | CYP2C19 | Unknown | c.681G > A | NA12878 |
| | | | 40bpDEL | |

Validation Overview:

The validation and verification are performed in multiple stages to evaluate accuracy, sensitivity, and specificity, as defined above.

Validation Plan:

Triplicate Sample Analysis: Coriell institute samples (NA12878) are prepared and analyzed in triplicate via the standard library preparation, as described in Example 5, and sequencing procedures, as described in Examples 5 and 6. The variant results acquired are compared to the human reference genome (GRCh37) and variant results are comprised using the ARCHER Pipeline. Variant results acquired are filtered and analyzed according to the data processing procedure. Final variant results are also analyzed using the GetRM database, as well.

Validation Criteria:

The following validation plan is performed per the established library preparation and MISEQ sequencer operation standard operating procedures, as described in Examples 5 and 6 respectively. Deviations to the established protocols are reported in the final validation report.

Overall MISEQ Sequencer Performance Requirements:

To be considered acceptable and included in the validation, all sample data collected are going to meet the following criteria: 80% bases are going to demonstrate a Q-score of at least 30 or above.

Sensitivity:

Sensitivity is evaluated through the analysis of samples with known genomic variants (true positives). Coriell sample NA12878 will be used to perform the initial sensitivity analysis. Additional Coriell samples will be analyzed, as needed, to reach the minimum variant requirement. Identified variants will be compared to high quality variant data available through the Get-RM database (including Sanger sequenced data) to accurately identify true positive and false negative results. Sensitivity will be calculated using the following formula: (TP/TP+FN)×100=% Sensitivity

Acceptance criteria: To be considered acceptable, at least 85% or higher sensitivity must be achieved with 95% confidence in the analyzed sample set.

Specificity:

Specificity will be evaluated through the analysis of samples with positions of no known variation within the targeted genomic region (true negative). Coriell sample NA12878 will be used to perform the initial specificity analysis. Additional samples will be analyzed, as needed, to reach the minimum variants requirement. As specified in the verification overview, additional samples with known variant(s) will be analyzed blindly to ascertain the sample based on the identified variant(s). Specificity will be calculated using the following formula:

(TN/TN+FP)×100=% Specificity.

Acceptance criteria: To be considered acceptable, at least 85% or higher specificity must be achieved with 95% confidence in the analyzed sample set.

Repeatability:

Repeatability is assessed through the library preparation and sequencing of three (N=3) replicate samples (uniquely indexed) in a single sequencing run. The Archer pipeline analysis is used to call all genomic variants in comparison to the human reference genome. All detected variants for each replicate sample are then compared to determine the closeness of results between replicate samples in the same sequencing run.

Acceptance criteria: All variant calls must agree 90% between replicate samples.

Reproducibility:

Reproducibility is assessed through the library preparation and sequencing of three (N=3) replicate samples (uniquely indexed) by multiple (>2) operator(s) on at least three processing days. The Archer pipeline analysis is used to call all genomic variants in comparison to the human reference genome. All detected variants for each replicate sample are then compared to determine the closeness of results between replicate samples across the multiple sequencing runs.

Acceptance criteria: All variant calls must agree 90% between replicate samples.

### Example 9

### Gene Sequencing Newborn Screening Report

Provided herein is an example of a gene sequencing newborn screening report.

TABLE 15
Client information for gene sequencing newborn screening

| Client Name* | MH | Specimen Type* | BLOOD, DBS |
|---|---|---|---|
| Parent Name | Not Provided | Date Specimen | 5JUN2014 |
| | | Collected* | |
| BG Sample ID* | BGTEST_19 | Date Specimen | 10JUN2014 |
| | | Received* | |
| Date of Birth* | 28DEC1984 | Report Date* | 7JUL2014 |
| Gender* | FEMALE | Referring Physician | Not Provided |
| Race/Ethnicity* | CAUCASIAN | Physician Phone | Not Provided |
| | | Number | |
| Birth Weight | Not Provided | Referring Facility | Not Provided |

*Required fields

The test performed is NGS newborn screening panel. The indications for test include, for example, identification of pathogenic or likely pathogenic genetic variants correlated with correlated with clinical metabolic conditions Newborn Screening Panel.

TABLE 16
Test results for a normal screen

| | Variant | Variant | Protein | | Chromosomal |
|---|---|---|---|---|---|
| Gene | Classification | Identified | Change | Zygosity | Location |
| ACAD8 | VOUS* | NM_014384.2: | p. Ser171Cys | Hetero | 134128923 |
| | | c. 512C > G | | | |
| ACADS | Pathogenic | NM_000017.2: | p. Gly209Ser | Hetero | 121176083 |
| | | c. 625G > A | | | |
| CPT1A | VOUS* | NM_001031847.2 | p. Ala275Thr | Hetero | 68562328 |
| | | c. 823G > A | | | |

*Variant of unknown significance

Interpretation Summary:

The test results for targeted gene sequencing and variant analysis of genes associated with newborn screening metabolic conditions, as exemplified in Table 16, did not identify pathogenic or likely pathogenic variants. On average, the targeted amplicons in this sample are covered at 87.0%. A report containing pathogenic or likely pathogenic variants indicates that a variant has been identified in the client sample that has been published as being associated with a specific condition. A report containing variant(s) identified as known pathogenic or likely pathogenic is not a diagnosis of a specific metabolic condition. It is recommended that any variants reported as VOUS be periodically reviewed to ensure the clinical significance has not changed. The laboratory does not provide medical advice and recommends that the client contact their physician or medical provider to discuss the results of this test.

In some embodiments, a physician, medical provider, or genetic counselor review the gene variant results reported in the laboratory report. Because the test is not a diagnostic test; a pathogenic or likely pathogenic gene variant is not a diagnosis of a medical condition.

A medical provider may request reanalysis of the genomic variant data for the presence of any pathogenic or likely pathogenic variants that is linked to disorders identified since the date of the report, as exemplified in Table 16 is linked to the client's phenotype based on currently available scientific information.

Test Method:

Genomic DNA is extracted from the submitted specimen and the NBS Panel reagent kit is used to target specific exonic regions of the specimen's genome. These targeted regions are sequenced using lumina sequencing technology with 2×150 bp paired-end reads. The DNA sequence is mapped to, and analyzed in comparison with, the published human genome build UCSC hg19 reference sequence. The targeted coding exons are assessed for average depth of coverage and data quality scores. All sequence variants are compared to the Gene Variant list to identify pathogenic and likely pathogenic variants based on current, published genomic data.

Table 17 exemplifies representative metrics from the client's targeted gene sequencing. Mean depth of coverage refers to the sequence mean read depth across the targeted region, defined as coding exons of the NBS panel reagent kit coding NBS Gene Panel. The quality score is logarithmically related to the base calling error probabilities. A quality score of 30 (Q30) is equivalent to the probability of an incorrect base in 1 in 1000 times, or a base call accuracy of 99.9%. Quality scores must meet the following minimum criteria (>80%>Q30).

TABLE 17
*The values below represent metrics from
the client's targeted gene sequencing:

| | |
|---|---|
| Mean Depth of Coverage¹ | TBR |
| Quality Score (% >Q30) | TBR % |

NBS Gene Panel: Targeted genes are correlated with metabolic conditions according to the American College of Medical Genetics (ACMG) recommendations. Exonic coding regions of the following genes are targeted in the NBS Gene Panel: ABCD1, ACAD8, ACADM, ACADS, ACADSB, ACADVL, ACAT1, ADA, AHCY, ARG1, ASL, ASS1, AUH, BCKDHA, BCKDHB, BTD, CBS, CFTR, CPT1A, CPT2, CYP21A2, DBT, DECR1, DLD, DNAJC19, DUOX2, EFTA, EFTB, EFTDH, FAH, GAA, GALC, GALE, GALK1, GALT, GBA, GCDH, GCH1, GJB2, GJB3, GJB6, GLA, GNMT, HADH, HADHA, HADHB, HBA1, HBA2, HBB, HLCS, HMGCKM HPD, HSD17B10, IDUA, IL2RG, IVD, MAT1A, MCCC1, MCCC2, MCEE, MLYCD, MMAA, MMAB, MMADHC, MTHFR, MTR, MTRR, MUT, NPC1, NPC2, OPA3, PAH, PAX8, PCBD1, PCCA, PCCB, PTS, QDPR, SLC22A5, SLC25A13, SLC25A20, ALC5A5, TAT, TAZ, TG, TPO, TSHB, TSHR. Limitations: Exonic coding regions of the targeted gene panel are sequenced and analyzed in the NBS Gene Panel; intronic regions are not targeted. The NGS Newborn Screen ensures, for example, 95% coverage of the targeted gene panel; due to inherent limitations in the biological testing system, a TBD % risk of false-negative or false-positive results exists within the test system. Some types of the genetic abnormalities, such as copy number changes, are detectable with the technologies performed by this analysis test. It is possible that the genomic coding region where a mutation exists in the proband is not captured using the current technologies and therefore is not detected. In this example, variants categorized as pathogenic or likely pathogenic are indicated on the client report, variants of unknown significance, likely benign, and benign are not reported. The variant(s) indicated on this example are identified based on current scientific information and are updated as new information becomes available.

### Example 10

### Sample Processing

Provided herein is an example of the sample handling and screening process the newborn screening. A blood sample is collected from a newborn within 24-48 hours of birth by a standard method such as heel puncture and collected on filter paper resulting in a dried blood spot (DBS) sample. The sample is shipped to a testing laboratory where the newborn screening method is conducted. Upon receipt of the sample by the testing facility, genomic DNA is extracted and purified from the DBS sample using a nucleic acid purification kit (e.g., CHARGESWITCH nucleic acid purification kit (Life Technologies)). Each sample of extracted and purified gDNA is quantified (e.g., using the QUBIT 2.0 fluorometer (Life Technologies) and a dsDNA High Sensitivity (HS) (Life Technologies) kit). gDNA concentrations are normalized prior to library preparation (e.g., to a concentration of 2 ng/μL). The library preparation process is then conducted, which includes the following steps: DNA fragmentation, A-tailing and end repair, adapter ligation, and amplification via PCR steps (e.g., using the lyophilized version of ARCHER DNA assay kit). Targeted sequencing is then conducted on the prepared library using a DNA sequencer to screen for one or more gene variants comprising, identifying a gene variant by sequencing at least one target region of each of gene PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 in genomic DNA from the newborn infant, wherein the sequencing does not include whole genome sequencing or whole exome sequencing. A report is provided to the newborn's parent or caregiver, which provides a list of gene variants identified in the sample, for those variants categorized as pathogenic or likely pathogenic are indicated on the client report.

### Example 11

### Newborn Screening Process—Uniform Screening Panel

Provided herein is an example of the process flow for the newborn screening. A blood sample is collected from a newborn within 24-48 hours of birth. The sample is shipped to a testing laboratory where the newborn screening method is conducted. The sample from the newborn is screened for one or more gene variants comprising, identifying a gene variant by sequencing at least one target region of each of gene PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 in genomic DNA from the newborn infant, wherein the sequencing does not include whole genome sequencing or whole exome sequencing. A report is provided to the newborn's parent or caregiver, which provides a list of gene variants identified in the sample, for those variants categorized as pathogenic or likely pathogenic are indicated on the client report.

### Example 12

### Newborn Screening Process—Core Conditions Panel

Provided herein is an example of the process flow for the newborn screening. A blood sample is collected from a newborn within 24-48 hours of birth. The sample is shipped to a testing laboratory where the newborn screening method is conducted. The sample from the newborn is screened for one or more gene variants comprising, identifying a gene variant by sequencing at least one target region of each of gene PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, ACAD8, MCCC1, MCCC2, HMGCL, HLCS, GCDH, SLC22A5, HADHB, ASS1, CBS, MTHFR, MTR, MTRR, MMADHC, PAH, FAH, DUOX2, PAX8, SLC5A5, TG, TPO, TSHB, TSHR, HBB, BTD, CFTR, GJB2, GJB3, GJB6, ADA, and IL2RG in genomic DNA from the newborn infant, wherein the sequencing does not include whole genome sequencing or whole exome sequencing. A report is provided to the newborn's parent or caregiver, which provides a list of gene variants identified in the sample, for those variants categorized as pathogenic or likely pathogenic are indicated on the client report.

### Example 13

### Newborn Screening Process—Core and Secondary Conditions Panel

Provided herein is an example of the process flow for the newborn screening. A blood sample is collected from a newborn within 24-48 hours of birth. The sample is shipped to a testing laboratory where the newborn screening method is conducted. The sample from the newborn is screened for one or more gene variants comprising, identifying a gene variant by sequencing at least one target region of each of gene PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, ACAD8, MCCC1, MCCC2, HMGCL, HLCS, GCDH, SLC22A5, HADHB, ASS1, CBS, MTHFR, MTR, MTRR, MMADHC, PAH, FAH, DUOX2, PAX8, SLC5A5, TG, TPO, TSHB, TSHR, HBB, BTD, CFTR, GJB2, GJB3, GJB6, ADA, IL2RG, MLYCD, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, and GALK1 in genomic DNA from the newborn infant, wherein the sequencing does not include whole genome sequencing or whole exome sequencing. A report is provided to the newborn's parent or caregiver, which provides a list of gene variants identified in the sample, for those variants categorized as pathogenic or likely pathogenic are indicated on the client report.

### Example 12

### Newborn Screening Process—Core, Secondary, and Added Conditions Panel

Provided herein is an example of the process flow for the newborn screening. A blood sample is collected from a newborn within 24-48 hours of birth. The sample is shipped to a testing laboratory where the newborn screening method is conducted. The sample from the newborn is screened for one or more gene variants comprising, identifying a gene variant by sequencing at least one target region of each of gene PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, ACAD8, MCCC1, MCCC2, HMGCL, HLCS, GCDH, SLC22A5, HADHB, ASS1, CBS, MTHFR, MTR, MTRR, MMADHC, PAH, FAH, DUOX2, PAX8, SLC5A5, TG, TPO, TSHB, TSHR, HBB, BTD, CFTR, GJB2, GJB3, GJB6, ADA, IL2RG, MLYCD, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, GALK1, GALC, GBA, NPC1, NPC2, GAA, GLA, IDUA, ABCD1, and NGLY1 in genomic DNA from the newborn infant, wherein the sequencing does not include whole genome sequencing or whole exome sequencing. A report is provided to the newborn's parent or caregiver, which provides a list of gene variants identified in the sample, for those variants categorized as pathogenic or likely pathogenic are indicated on the client report.

The examples and embodiments described herein are for illustrative purposes only and various modifications or changes suggested to persons skilled in the art are to be included within the spirit and purview of this application and scope of the appended claims.

1. A method for early detection in newborn infants of one or more gene variants associated with an asymptomatic disease, comprising:
obtaining a genomic DNA containing sample from the newborn infant to generate a genomic library, wherein each fragmented genomic DNA from the genomic library comprises an adaptor, wherein the sample is from a newborn infant between 0 and 72 hours after birth, and wherein the infant is asymptomatic for a disease or disorder;
performing a plurality of DNA sequencing reactions on the genomic library to determine the DNA sequence of at least one target region of each of gene PCCA, PCCB, MUT, MMAA, MMAB, MMADHC, MCEE, IVD, ACAT1, ACADM, ACADVL, HADHA, ASL, BCKDHA, BCKDHB, DBT, DLD, CYP21A2, GALT, and ACAD8 in the genomic DNA; and
screening for a gene variant from the sequenced target regions of each gene to identify gene variants present in the genomic DNA,
wherein the sequencing does not include whole genome sequencing or whole exome sequencing.

2. The method of claim 1, wherein the one or more gene variants are associated with one or more diseases or disorders.

3. (canceled)

4. The method of claim 1, wherein the method is completed in less than 96 hours.

5. The method of claim 1, further comprising sequencing at least one target region of one or more genes selected from the group consisting of MCCC1, MCCC2, HMGCL, HLCS, GCDH, SLC22A5, HADHB, ASS1, CBS, MTHFR, MTR, MTRR, MMADHC, PAH, FAH, DUOX2, PAX8, SLC5A5, TG, TPO, TSHB, TSHR, HBB, BTD, CFTR, GJB2, GJB3, GJB6, ADA, and IL2RG.

6. The method of claim 5, further comprising sequencing at least one target region of one or more genes selected from the group consisting of MLYCD, ACADSB, AUH, DNAJC19, OPA3, TAZ, HSD17B10, ACADS, HADH, ETFA, ETFB, ETFDH, DECR1, CPT1A, CPT2, SLC25A20, ARG1, SLC25A13, AHCY, GNMT, MAT1A, PAH, GCH1, PCBD1, PTS, QDPR, TAT, HPD, HBA1, HBA2, HBB, GALE, GALK1, GALC, GBA, NPC1, NPC2, GAA, GLA, IDUA, ABCD1, and NGLY1.

7. The method of claim 1, wherein two or more target regions for each gene are sequenced.

8. The method of claim 6, wherein the gene variants are selected from among gene variants listed in Table 5.

9. (canceled)

10. The method of claim 1, wherein the variant is identified using a computer software module.

11. The method of claim 1, wherein the newborn infant does not exhibit symptoms of a metabolic disease or condition.

12. The method of claim 1, further comprising providing a report comprising a list of variants identified in the genomic DNA.

13. The method of claim 12, wherein the report comprises a list of diseases or disorders associated with each variant.

14. The method of claim 2, wherein the method further comprises selecting the infant for diagnostic analysis of the disease or disorder if a gene variant is identified.

15. The method of claim 6, wherein the one or more gene variants are associated with one or more diseases or disorders selected from the group consisting of metabolic disorder, an endocrine disorder, or a hemoglobin disorder.

16. The method of claim 15, wherein the metabolic disorder is an organic acid disorder, a fatty acid oxidation disorder, or an amino acid disorder.

17. The method of claim 15, wherein the wherein the metabolic disorder is propionic acidemia (PROP), methylmalonic acidemia (MUT), isovaleric acidemia (IVA), 3-methylcrotonyl-CoA carboxylase deficiency (3-MCC), 3-hydroxy-3-methylglutaryl-CoA lyase deficiency (HMG), multiple carboxylase deficiency (MCD), beta-ketothiolase deficiency (βKT), glutaric acidemia type I (GA1), primary carnitine deficiency (CUD), medium-chain acyl-CoA dehydrogenase (MCAD) deficiency, very long-chain acyl-CoA dehydrogenase (VLCAD) deficiency, trifunctional protein deficiency (TFP), long chain 3-hydroxyacyl-CoA dehydrogenase (LCHAD) deficiency, argininosuccinic aciduria (ASA), citrullinemia (CIT) type I, maple syrup urine disease (MSUD), homocystinuria (HCY), phenylketonuria (PKU), or tyrosinemia (TYR I, II, III).

18. The method of claim 15, wherein the endocrine disorder is congenital hypothyroidism (CH) or 21-hydroxylase deficiency (CAH).

19. The method of claim 15, wherein the hemoglobin disorder is sickle cell disease, metheglobinemia, beta-globin type, or beta thalassemia.

20. The method of claim 6, wherein the disease or disorder is biotinidase deficiency (BIOT), cystic fibrosis (CF), galactosemia type I, hearing loss, severe combined immunodeficiency (SCID), or X-linked severe combined immunodeficiency (SCID), malonyl-CoA decarboxylase deficiency (MAL), isobutyryl-CoA dehydrogenase (IBD) deficiency, 2-methylbutyryl-CoA dehydrogenase deficiency, 3-methylglutaconic aciduria (3MGA) type I, 3-methylglutaconic aciduria (3MGA) type V, 3-hydroxy-2-methylbutyryl-CoA dehydrogenase deficiency (2M3HBA), short-chain acyl-CoA dehydrogenase (SCAD) deficiency, 3-hydroxyacyl-CoA dehydrogenase deficiency (M/SCHAD), glutaric acidemia type II (GA2), glutaric acidemia type II (GA2), carnitine palmitoyltransferase I deficiency (CPT IA), carnitine palmitoyltransferase II deficiency (CPT II), carnitine-acylcarnitine translocase (CACT), arginase deficiency (ARG), citrullinemia type II (CIT II), hypermethioninemia (MET), disorders of biopterin regeneration, tyrosinemia (TYR I, II, III), alpha thalassemia (hemoglobin disorder-Var-Hb), galactosemia type II, galactosemia type III, X-linked adrenoleukodystrophy, adrenomyeloneuropathy, Addison disease (X-ALD), 2,4 dienoyl-CoA reductase deficiency, Pompe disease (GAA deficiency), Krabbe disease, Gaucher disease (types I, II, & III), Fabry disease, mucopolysaccharidosis type I (MPS I), congenital disorder of deglycosylation type 1v, Niemann-Pick disease (type C1), or Niemann-Pick disease (type C2).
